(19) United States
(12) Patent Application Publication
(10) Pub. No.: US 2020/0237800 A1
(43) Pub. Date: Jul. 30, 2020

(54) METHODS AND COMPOSITIONS FOR THE TREATMENT OF INFLAMMATORY DISEASE

(71) Applicant: THE BRIGHAM AND WOMEN'S HOSPITAL, INC., Boston, MA (US)

(72) Inventors: Masanori AIKAWA, Chestnut Hill, MA (US); Jian-Guo WANG, Chestnut Hill, MA (US); Sasha A. SINGH, Boston, MA (US)

(73) Assignee: THE BRIGHAM AND WOMEN'S HOSPITAL, INC., Boston (US)

(21) Appl. No.: 16/754,571

(22) PCT Filed: Oct. 9, 2018
(86) PCT No.: PCT/US2018/054950

§ 371 Date: Apr. 8, 2020

Related U.S. Application Data
(60) Provisional application No. 62/570,405, filed on Oct. 10, 2017.

(51) Int. Cl. A61K 31/7105; A61K 31/713; C12N 15/113; A61P 37/02; G01N 33/68

(52) U.S. Cl. CPC A61K 31/7105; A61K 31/713; G01N 2800/26; A61P 37/02; G01N 33/6875; C12N 15/113

(57) ABSTRACT

Described herein are methods and compositions related to the treatment or prevention of an inflammatory disease via administration of an agent that inhibits a long noncoding RNA (Inc-RNA) expressed in a macrophage to a subject in need thereof. In one embodiment, the Inc-RNA is lnc-FAM164A1. Another aspect herein provides a method of treating or preventing an inflammatory disease via administration of an agent that inhibits a protein bound to a Inc-RNA, e.g., ACLY, to a subject in need thereof.

### CROSS-REFERENCE TO RELATED APPLICATIONS

This Application is a 35 U.S.C. § 371 National Phase Entry Application of International Application No. PCT/US18/54950 filed Oct. 9, 2018, which designates the U.S. and claims benefit under 35 U.S.C. § 119(e) of U.S. Provisional Application No. 62/570,405 filed Oct. 10, 2017 the contents of which are incorporated herein by reference in their entireties.

### SEQUENCE LISTING

The instant application contains a Sequence Listing which has been submitted electronically in ASCII format and is hereby incorporated by reference in its entirety. Said ASCII copy, created on Jul. 24, 2018, is named 002806-089891WOPT_SL.txt and is 64,938 bytes in size.

### TECHNICAL FIELD

The technology described herein relates to methods and compositions for treating inflammatory disease and cancer.

### BACKGROUND

Long noncoding RNAs (lnc-RNAs, >200 nucleotides), play unsuspected roles in numerous biological processes and human diseases including embryonal development, tumor growth and metastasis, cardiometabolic disorders as well as inflammation. Several lnc-RNAs have been characterized as important regulators among the differentiation of immune cells, such as erythroid cells, T-lymphocytes, dendritic cells and monocytes/macrophages.⁶⁻⁹ However, their biological function in immunity remains largely undetermined. Human and murine macrophages express thousands of lnc-RNAs, but only several lnc-RNAs have been characterized in modulating the activation of macrophages.¹⁵⁻²⁴ Therefore, more lnc-RNAs need to be identified and tested for their function. Specific lnc-RNAs that regulate macrophage activation can function as a novel target for the treatment and prevention of inflammatory diseases, e.g., autoimmune disorders, or by leveraging lnc-RNAs for their use in cancer immunotherapies.

### SUMMARY

Described herein are data that show that human lnc-FAM164A1 enhances the expression of pro-inflammatory cytokines through NF-κB signaling in LPS-activated macrophages and identifies lnc-FAM164A1 as a therapeutic target for inflammatory disease and cancer. Specifically, siRNA-directed knockdown of lnc-FAM164A1 reduces the expression of inflammation-associated cytokines (e.g., suppressed the LPS-induced expression of pro-inflammatory cytokines such as CCL2/MCP-1, IL-6 and TNF-α) in a mouse, reducing inflammation in an exotoxemia mouse model. Further work described herein identified three proteins bound to lnc-FAM164A1: ATP-citrate synthase (ACLY); Filaggrin-2 (Flg2); or Splicing factor, proline-and-glutamine-rich (SFPQ). Work described herein show that ATP-citrate synthase (ACLY) is a lnc-FAM164A1-associated protein. Further, ACLY silencing decreases the expression of CCL2 and TNF-α induced by enforced expression of lnc-FAM164A1 in vitro.

Accordingly, one aspect provided herein provides a method of treating or preventing an inflammatory disease in a subject comprising administering to a subject an effective amount of an agent that inhibits a long noncoding RNA(lnc-RNA) expressed in a macrophage. In one embodiment, the lnc-RNA is selected from the group consisting of AL078621.3; RP11-536K7.5; RP11-143M1.3; RP11-561O23.5; AC10980.2; P2RX7; LOC440896; D63785; RP11-58A12.2; RP11-79H23.3 (lnc-FAM164A1); and RP13-452N2.1.

Another aspect provided herein provides a method of treating or preventing an inflammatory disease in a subject comprising administering to a subject an effective amount of an agent that inhibits lnc-FAM164A1.

In one embodiment of any aspect, the inflammatory disease is selected from the group consisting of: endotoxemia, atherosclerotic vascular disease, heart valve disease (e.g., aortic valve disease or mitral valve disease), heart failure, autoimmune disease, osteoporosis, ectopic calcification, brain damage after stroke, obesity, fatty liver disease, diabetes, Gaucher's disease, sepsis, kidney dysfunction, kidney failure, and cancer.

In one embodiment of any aspect, the inflammatory disease occurs in response to a tissue or cell transplantation.

In one embodiment of any aspect, the inflammatory disease is acute or chronic.

In one embodiment of any aspect, the agent is selected from the group consisting of a small molecule, an antibody, a peptide, a genome editing system, an antisense oligonucleotide, and an RNA interference (RNAi), an antisense RNA, an RNA decoy molecule, an RNAaptamer, or an inhibitory polypeptide. Exemplary RNAi include, but are not limited to, microRNA, an siRNA, or a shRNA

In one embodiment of any aspect, the agent is a vector that encodes the agent.

In one embodiment of any aspect, the vector is non-integrative or integrative.

Exemplary non-integrative vectors include, but are not limited to, an episomal vector, an EBNA1 vector, a minicircle vector, a non-integrative adenovirus, a non-integrative RNA, and a Sendai virus.

In one embodiment of any aspect, the vector is a lentiviral vector.

In one embodiment of any aspect, the lnc-RNA is inhibited in a target cell. In one embodiment of any aspect, the lnc-FAM164A1 is inhibited in a target cell.

In one embodiment of any aspect, the target cell is a mammalian cell. In one embodiment of any aspect, the target cell is a macrophage (e.g. an inactivated or activated macrophage), T cell, dendritic cells, B cell, natural killer cell, or neutrophil. In one embodiment, the macrophage activation macrophage is uncontrolled activation.

In one embodiment of any aspect, the subject is a mammal. In one embodiment of any aspect, the subject is a humn.

In one embodiment of any aspect, the inhibiting is reducing the level and/or activity.

In one embodiment of any aspect, the effective amount of the agent reduces the level of lnc-RNA by at least 50%, at least 60%, at least 70%, at least 80%, at least 90%, or more as compared to an appropriate control.

In one embodiment of any aspect, the effective amount of the agent reduces the activity of lnc-RNA by at least 50%, at least 60%, at least 70%, at least 80%, at least 90%, or more as compared to an appropriate control.

In one embodiment of any aspect, the effective amount of the agent reduces the level of lnc-FAM164A1 by at least 50%, at least 60%, at least 70%, at least 80%, at least 90%, or more as compared to an appropriate control.

In one embodiment of any aspect, the effective amount of the agent reduces the expression of lnc-FAM164A1 by at least 50%, at least 60%, at least 70%, at least 80%, at least 90%, or more as compared to an appropriate control.

In one embodiment of any aspect, the subject has previously been diagnosised with having an inflammatory disease.

In one embodiment of any aspect, the subject has not previously been diagnosed with having an inflammatory disease.

In one embodiment of any aspect, the subject exhibits at least one risk factor of developing an inflammatory disease.

In one embodiment of any aspect, the method further comprises, prior to adminisitering, diagnosing a subject as having an inflammatory disease.

In one embodiment of any aspect, the method further comprises, prior to administering, receiving the results of an assay that diagnoses a patient as having an inflammatory disease.

In one embodiment of any aspect, the agent decreases expression of CCL2 and IL-6 in a plasma cell and/or a peritoneal cell.

In one embodiment of any aspect, the agent decreases NFκB signaling.

Another aspect described herein a method of treating or preventing an inflammatory disease in a subject comprising administering to a subject an effective amount of an agent that inhibits a protein bound to a lnc-RNA. In one embodiment, the protein bound to the lnc-RNA is ATP-citrate synthase (ACLY); Filaggrin-2 (Flg2); or Splicing factor, proline-and-glutamine-rich (SFPQ). In one embodiment, the protein bound to the lnc-RNA is ACLY.

In one embodiment of any aspect, the agent decreases the expression of CCL2 and TNF-α in the subject.

In various embodiments of this aspect, or any aspect, the agent can inhibit the activity or levels of the protein. The agent can inhibit the binding of the protein to a lnc-RNA, for example, lnc-FAM164A1.

In one embodiment of any aspect, the effective amount of the agent reduces the level of the protein by at least 50%, at least 60%, at least 70%, at least 80%, at least 90%, or more as compared to an appropriate control.

In one embodiment of any aspect, the effective amount of the agent reduces the activity of the protein by at least 50%, at least 60%, at least 70%, at least 80%, at least 90%, or more as compared to an appropriate control.

In one embodiment of any aspect, the effective amount of the agent reduces the binding of the protein to a lnc-RNA, for example, lnc-FAM164A1, by at least 50%, at least 60%, at least 70%, at least 80%, at least 90%, or more as compared to an appropriate control.

Another aspect described herein provides a method of treating an inflammatory disease in a subject comprising (a) receiving results of an assay that measures a level of a lnc-RNA expressed in a macrophage in a biological sample; (b) comparing the level of the lnc-RNA to a reference level; (c) identifying a subject as having an inflammatory disease if the level of the lnc-RNA is significantly increased as compared to the reference level; and (d) administering to the subject having an inflammatory disease an agent that inhibits the lnc-RNA, or a composition comprising an agent that inhibits the lnc-RNA.

Another aspect described herein provides a method of treating an inflammatory disease in a subject comprising (a) measuring a level of a lnc-RNA expressed in a macrophage in a biological sample; (b) comparing the level of the lnc-RNA to a reference level; (c) identifying a subject as having an inflammatory disease if the level of the lnc-RNA is significantly increased as compared to the reference level; and (d) administering to the subject having an inflammatory disease an agent that inhibits the lnc-RNA, or a composition comprising an agent that inhibits the lnc-RNA. In one embodiment of any aspect, the method further comprises, prior to step a), obtaining a biological sample from the subject.

Another aspect described herein provides a pharmaceutical composition comprising an agent that inhibits a lnc-RNA expressed in a macrophage and a pharmaceutically acceptable carrier.

Another aspect described herein provides a pharmaceutical composition comprising an agent that inhibits lnc-FAM164A1 and a pharmaceutically acceptable carrier.

Another aspect described herein provides a pharmaceutical composition comprising an agent that inhibits a protein bound to a lnc-RNA and a pharmaceutically acceptable carrier. In one embodiment, the protein bound to the lnc-RNA is ATP-citrate synthase (ACLY); Filaggrin-2 (Flg2); or Splicing factor, proline-and-glutamine-rich (SFPQ). In one embodiment, the protein bound to the lnc-RNA is ACLY.

Another aspect described herein provides a method of identifying a subject having sepsis, the method comprising (a) measuring a level of a lnc-FAM164A1 in a biological sample; (b) comparing the level of the lnc-FAM164A1 to a reference level; (c) identifying a subject as having sepsis if the level of the lnc-RNA is significantly increased as compared to the reference level; and (d) administering to the subject having sepsis a therapeutic to treat sepsis. In one embodiment, the method further comprises, prior to step (a), obtaining a biological sample from the subject. In one embodiment, the biological sample is a blood sample.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed technology, because the scope of the technology is limited only by the claims. Unless otherwise defined, all technical and scientific terms used herein have the same meaning as commonly understood by one of ordinary skill in the art to which this technology belongs. If there is an apparent discrepancy between the usage of a term in the art and its definition provided herein, the definition provided within the specification shall prevail.

Definitions of common terms in immunology and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with an inflammatory disease, e.g., atherosclerotic vascular disease, vein graft failure, arteriovenous fistula failure, heart valve disease, heart failure, autoimmune disease, osteoporosis, ectopic calcification, brain damage after stroke, obesity, fatty liver disease, diabetes, Gaucher's disease, sepsis, kidney dysfunction, kidney failure, and cancer. The term “treating” includes reducing or alleviating at least one adverse effect or symptom of an inflammatory disease, for example, fever, chest pain, shorness of breath, or nausea. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein “preventing” or “prevention” refers to any methodology where the disease state does not occur due to the actions of the methodology (such as, for example, administration of an agent as described herein). In one aspect, it is understood that prevention can also mean that the disease is not established to the extent that occurs in untreated controls. Accordingly, prevention of a disease encompasses a reduction in the likelihood that a subject can develop the disease, relative to an untreated subject (e.g. a subject who is not treated with the methods or compositions described herein).

As used herein, the term “administering,” is used in the context of the placement of an agent described herein, into a subject or biological sample, by a method or route which results in at least partial localization of the introduced agent at a desired site, such as the bloodstream or a region thereof, such that a desired effect(s) is produced (e.g., decreased macrophage activation and inflammation). The agent described herein can be administered by any appropriate route which results in delivery to a desired location in the subject. In some embodiments, the term “administering” refers to the administration of a pharmaceutical composition comprising one or more agents or cells. The administering can be done by direct injection (e.g., directly administered to a target cell), subcutaneous injection, muscular injection, oral, or nasal delivery to the subject in need thereof. Administering can be local or systemic.

The terms “patient”, “subject” and “individual” are used interchangeably herein, and refer to an animal, particularly a human, to whom treatment, including prophylactic treatment is provided. The term “subject” as used herein refers to human and non-human animals. The term “non-human animals” and “non-human mammals” are used interchangeably herein includes all vertebrates, e.g., mammals, such as non-human primates, (particularly higher primates), sheep, dog, rodent (e.g. mouse or rat), guinea pig, goat, pig, cat, rabbits, cows, and non-mammals such as chickens, amphibians, reptiles etc. In one embodiment, the subject is human. In another embodiment, the subject is an experimental animal or animal substitute as a disease model. In another embodiment, the subject is a domesticated animal including companion animals (e.g., dogs, cats, rats, guinea pigs, hamsters etc.). A subject can have previously received a treatment for an inflammatory disease, or has never received treatment for an inflammatory disease. A subject can have previously been diagnosed with having an inflammatory disease, or has never been diagnosed with an inflammatory disease. A subject can exhibit at least one risk factor of an inflammatory disease.

The subject can be initially diagnosed by a licensed physician and/or authorized medical practitioner, and a regimen for prophylactic and/or therapeutic treatment via a method described herein can be suggested, recommended or prescribed. Thus, in some embodiments, the method comprises identifying a subject as in need of treatment for an inflammatory disease.

Animal models that are reliable indicators of inflammatory disease, that include but are not limited to endotoxemia, atherosclerotic vascular disease, vein graft failure, arteriovenous fistula failure, heart valve disease, heart failure, autoimmune disease, osteoporosis, ectopic calcification, brain damage after stroke, obesity, fatty liver disease, diabetes, Gaucher's disease, sepsis, kidney dysfunction, kidney failure, and cancer.

The term “agent” as used herein means any compound or substance such as, but not limited to, a small molecule, an antibody, a peptide, a genome editing system, an antisense oligonucleotide, and an RNA interference (RNAi), an antisense RNA, an RNA decoy molecule, an RNAaptamer, or an inhibitory polypeptide. list here] etc. An “agent” can be any chemical, entity or moiety, including without limitation synthetic and naturally-occurring proteinaceous and non-proteinaceous entities. In some embodiments, an agent is a small molecule, an antibody, a peptide, a genome editing system, an antisense oligonucleotide, and an RNA interference (RNAi), an antisense RNA, an RNA decoy molecule, an RNAaptamer, or an inhibitory polypeptide, and modifications and combinations thereof etc. In certain embodiments, agents are small molecule having a chemical moiety. For example, chemical moieties included unsubstituted or substituted alkyl, aromatic, or heterocyclyl moieties including macrolides, leptomycins and related natural products or analogues thereof. Compounds can be known to have a desired activity and/or property, or can be selected from a library of diverse compounds.

The agent can be a molecule from one or more chemical classes, e.g., organic molecules, which can include organometallic molecules, inorganic molecules, genetic sequences, etc. Agents can also be fusion proteins from one or more proteins, chimeric proteins (for example domain switching or homologous recombination of functionally significant regions of related or different molecules), synthetic proteins or other protein variations including substitutions, deletions, insertion and other variants.

The term “decrease”, “reduced”, “reduction”, or “inhibit” are all used herein to mean a decrease by a statistically significant amount. In some embodiments, “decrease”, “reduced”, “reduction”, or “inhibit” typically means a decrease by at least 10% as compared to an appropriate control (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99% , or more as compared to an appropriate control. As used herein, “reduction” or “inhibition” does not encompass a complete inhibition or reduction as compared to an appropriate control. “Complete inhibition” is a 100% inhibition as compared to an appropriate control.

The terms “increase”, “enhance”, or “activate” are all used herein to mean an increase by a reproducible statistically significant amount. In some embodiments, the terms “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, a 20 fold increase, a 30 fold increase, a 40 fold increase, a 50 fold increase, a 6 fold increase, a 75 fold increase, a 100 fold increase, etc. or any increase between 2-fold and 10-fold or greater as compared to an appropriate control. In the context of a marker, an “increase” is a reproducible statistically significant increase in such level.

As used herein, an “appropriate control” refers to an untreated, otherwise identical cell or population (e.g., a patient who was not administered an agent described herein, or was administered by only a subset of agents described herein, as compared to a non-control cell).

As used herein, a “reference level” refers to a normal, otherwise unaffected cell population or tissue (e.g., a biological sample obtained from a healthy subject, or a biological sample obtained from the subject at a prior time point, e.g., a biological sample obtained from a patient prior to being diagnosed with an inflammatory disease, or a biological sample that has not been contacted with an agent disclosed herein).

As used herein, the term “long-noncoding RNA” or “lnc-RNA” refers to an RNA transcript longer than 200 nucleotides that is not translated into protein. Long noncoding RNAs are involved in a number of cellular functions such as gene regulation, chromatin regulation, scaffolds for two or more proteins, or guides for the proper localization of specific protein complexes (See, e.g., Rin and Chang et al Annu Rev Biochem. 2012, which is incorporated herein by reference in its entirety).

As used herein, the term “lnc-FAM164A1” or “ENSG00000261618.1” or “OTTHUMG00000173421.1” refers to a long-noncoding RNA of about 2994 base pairs that is located on chromosome 8 (position 79,749,764 to 79,752,757) close to the protein coding gene FAM164A. In macrophages, lnc-FAM164A1 enhances the secretion of inflammatory cytokines. Sequences for lnc-FAM164A1, are known for a number of species, e.g., human lnc-FAM164A1 (Name: AC083837.1-201), transcript (e.g., TRANSCRIPT ID: ENST00000565297.1 or Ensembl Transcript chromosome: GRCh37:8:1:146364022:1). Long noncoding RNA, lnc-FAM164A1, can refer to human lnc-FAM164A1, including naturally occurring variants, molecules, and alleles thereof. The long non-coding RNA, lnc-FAM164A1, refers to the mammalian lnc-FAM164A1 of, e.g., mouse, rat, rabbit, dog, cat, cow, horse, pig, and the like. The nucleic sequence of SEQ ID NO: 1 comprises the nucleic sequence which encodes lnc-FAM164A1.

As used herein, the term “lnc-FAM164A1 activity” refers to the cellular functions of lnc-FAM164A1, for example, lnc-FAM164A1 enhances the expression of pro-inflammatory cytokines through NF-κB signaling in LPS-activated macrophages. For example, an increase in lnc-FAM164A1 activity can refer to the increase in phagocytosis and cytokine secretion by a cell. Non-limiting examples of cytokines secreted by activated macrophages through the NF-κB pathway include, but are not limited to, IL-1β, IL-6, and TNF-α. However, it is contemplated that other cytokines can also be measured to determine the level of lnc-FAM164A1 activity.

Methods and compositions described herein require that the ACLY is targeted. As used herein, “ATP Citrate Lyase (ACLY)” refers to the primary enzyme responsible for the synthesis of cytosolic acetyl-CoA in many tissues. Sequences for ACLY, also known as ACL, ATPCL, and CLATP, are known for a number of species, e.g., human ACLY (NCBI Gene ID: 47) polypeptide (e.g., NCBI Ref Seq NP_001087.2) and mRNA (e.g., NCBI Ref Seq NM_001096.2). ACLY can refer to human ACLY, including naturally occurring variants, molecules, and alleles thereof. ACLY refers to the mammalian ACLY of, e.g., mouse, rat, rabbit, dog, cat, cow, horse, pig, and the like. The nucleic sequence of SEQ ID NO: 3 comprises the nucleic sequence which encodes ACLY.

Methods and compositions described herein require that the Flg2 is targeted. As used herein, “filaggrin family member 2 (Flg2)” refers to a gene that encodes the protein required for cornification in skin. Mutations in this gene are often associated with skin disease. Sequences for Flg2, also known as IFPS and PSS6, are known for a number of species, e.g., human Flg2 (NCBI Gene ID: 388698) polypeptide (e.g., NCBI Ref Seq NP_001014364.1) and mRNA (e.g., NCBI Ref Seq NM_001014342.2). Flg2 can refer to human Flg2, including naturally occurring variants, molecules, and alleles thereof. Flg2 refers to the mammalian Flg2 of, e.g., mouse, rat, rabbit, dog, cat, cow, horse, pig, and the like. The nucleic sequence of SEQ ID NO: 38 comprises the nucleic sequence which encodes Flg2 (NM 001014342.2 Homo sapiens filaggrin family member 2 (FLG2), mRNA).

Methods and compositions described herein require that the SFPQ is targeted. As used herein, “Splicing factor proline and glutamine rich (SFPQ)” refers to splice factor ubiquitously expressed in many tissues. Sequences for SFPQ, also known as PSF, POMP100, and PPP1R140, are known for a number of species, e.g., human SFPQ (NCBI Gene ID: 6421) polypeptide (e.g., NCBI Ref Seq NP_005057.1) and mRNA (e.g., NCBI Ref Seq NM_005066.2). SFPQ can refer to human SFPQ, including naturally occurring variants, molecules, and alleles thereof. SFPQ refers to the mammalian SFPQ of, e.g., mouse, rat, rabbit, dog, cat, cow, horse, pig, and the like. The nucleic sequence of SEQ ID NO: 39 comprises the nucleic sequence which encodes SFPQ (mRNA (cDNA clone MGC:57191 IMAGE:5262885), complete cds).

The term “inflammatory disease” can be used interchangeably with “inflammation” to refer to diseases and conditions associated with inflammation which can include but are not limited to: (1) inflammatory or allergic diseases such as systemic anaphylaxis or hypersensitivity responses, drug allergies, insect sting allergies; inflammatory bowel diseases, such as Crohn's disease, ulcerative colitis, ileitis and enteritis; vaginitis; psoriasis and inflammatory dermatoses such as dermatitis, eczema, atopic dermatitis, allergic contact dermatitis, urticaria; vasculitis; spondyloarthropathies; scleroderma; respiratory allergic diseases such as asthma, allergic rhinitis, hypersensitivity lung diseases, and the like, (2) autoimmune diseases, such as arthritis (rheumatoid and psoriatic), osteoarthritis, multiple sclerosis, systemic lupus erythematosus, diabetes mellitus, glomerulonephritis, and the like, (3) graft rejection (including allograft rejection and graft-v-host disease) or rejection of an engineered tissue, and (4) other diseases in which undesired inflammatory responses are to be inhibited including infectious diseases, myositis, inflammatory CNS disorders such as stroke and closed-head injuries, neurodegenerative diseases, Alzheimer's disease, encephalitis, meningitis, osteoporosis, gout, hepatitis, nephritis, sepsis, sarcoidosis, conjunctivitis, otitis, chronic obstructive pulmonary disease, sinusitis, Bechet's syndrome, endotoxemia, atherosclerotic vascular disease, coronary artery disease, stent restenosis, carotid metabolic disease, stroke, acute myocardial infarction, heart failure, peripheral arterial disease, limb ischemia, vein graft failure, or a AV fistula failure.

As used herein, the term “immunotherapy” refers to the treatment of a disease via the stimulation, induction, subversion, mimicry, enhancement, augmentation or any other modulation of a subject's immune system to elicit or amplify adaptive or innate immunity (actively or passively) against cancerous or otherwise harmful proteins, cells or tissues. Immunotherapies (i.e., immunotherapeutic agents) include cancer vaccines, immunomodulators, “antibody-based immunotherapies” or monoclonal antibodies (e.g., humanized monoclonal antibodies), immunostimulants, cell-based therapies such as adoptive T-cell therapies, natural killer cell, macrophage, or dendritic cell immunotherapies or dendritic cell vaccines, and viral therapies, whether designed to treat existing cancers or prevent the development of cancers or for use in the adjuvant setting to reduce likelihood of recurrence of cancer.

As used herein, the term “small molecule” refers to a chemical agent which can include, but is not limited to, a peptide, a peptidomimetic, an amino acid, an amino acid analog, a polynucleotide, a polynucleotide analog, an aptamer, a nucleotide, a nucleotide analog, or an organic or inorganic compound (e.g., including heterorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other forms of such compounds.

As used herein, the term “nucleic acid” includes one or more types of: polydeoxyribonucleotides (containing 2-deoxy-D-ribose), polyribonucleotides (containing D-ribose), and any other type of polynucleotide that is an N-glycoside of a purine or pyrimidine base, or modified purine or pyrimidine bases (including abasic sites). The term “nucleic acid,” as used herein, also includes polymers of ribonucleosides or deoxyribonucleosides that are covalently bonded, typically by phosphodiester linkages between subunits, but in some cases by phosphorothioates, methylphosphonates, and the like. “Nucleic acids” include single- and double-stranded DNA, as well as single- and double-stranded RNA. Exemplary nucleic acids include, without limitation, gDNA; hnRNA; mRNA; rRNA, tRNA, micro RNA (miRNA), small interfering RNA (siRNA), small nucleolar RNA (snORNA), small nuclear RNA (snRNA), and small temporal RNA (stRNA), and the like, and any combination thereof

The term “RNAi” as used herein refers to interfering RNA, or RNA interference molecules are nucleic acid molecules or analogues thereof for example RNA-based molecules that inhibit gene expression. RNAi refers to a means of selective post-transcriptional gene silencing. RNAi can result in the destruction of specific mRNA, or prevents the processing or translation of RNA, such as mRNA. Non-limiting examples of RNAi include RNAi is an silencing RNA (siRNA), an endoribonuclease-prepared si RNA (esiRNA), a short hairpin RNA (shRNA), or a microRNA (miRNA).

The term “shRNA” as used herein refers to short hairpin RNA which functions as RNAi and/or siRNA species but differs in that shRNA species are double stranded hairpin-like structure for increased stability.

As used herein, the term “aptamer” or “RNA aptamer” means a single-stranded, partially single-stranded, partially double-stranded or double-stranded nucleotide sequence capable of specifically recognizing a selected non-oligonucleotide molecule or group of molecules. Accordingly, aptamers are nucleic acid or peptide molecules that bind to a particular molecule of interest with high affinity and specificity (Tuerk and Gold, Science 249:505 (1990); Ellington and Szostak, Nature 346:818 (1990)). DNA or RNA aptamers have been successfully produced which bind many different entities from large proteins to small organic molecules. See Eaton, Curr. Opin. Chem. Biol. 1:10-16 (1997), Famulok, Curr. Opin. Struct. Biol. 9:324-9(1999), and Hermann and Patel, Science 287:820-5 (2000). Aptamers can be RNA or DNA based. Methods for selecting aptamers for binding to a molecule are widely known in the art and easily accessible to one of ordinary skill in the art. Generally, aptamers are engineered through repeated rounds of in vitro selection or equivalently, SELEX (systematic evolution of ligands by exponential enrichment) to bind to various molecular targets such as small molecules, proteins, nucleic acids, and even cells, tissues and organisms. The aptamer can be prepared by any known method, including synthetic, recombinant, and purification methods, and can be used alone or in combination with other aptamers specific for the same target. Further, as described more fully herein, the term “aptamer” specifically includes “secondary aptamers” containing a consensus sequence derived from comparing two or more known aptamers to a given target. Aptamers can include, without limitation, defined sequence segments and sequences comprising nucleotides, ribonucleotides, deoxyribonucleotides, nucleotide analogs, modified nucleotides and nucleotides comprising backbone modifications, branchpoints and nonnucleotide residues, groups or bridges. In some embodiments, the aptamer recognizes the non-oligonucleotide molecule or group of molecules by a mechanism other than Watson-Crick base pairing or triplex formation.

As used herein, the terms “proteins” and “peptides” are used interchangeably herein to designate a series of amino acid residues connected to the other by peptide bonds between the alpha-amino and carboxy groups of adjacent residues. The terms “protein”, and “peptide”, which are used interchangeably herein, refer to a polymer of protein amino acids, including modified amino acids (e.g., phosphorylated, glycated, etc.) and amino acid analogs, regardless of its size or function. Although “protein” is often used in reference to relatively large polypeptides, and “peptide” is often used in reference to small polypeptides, usage of these terms in the art overlaps and varies. The term “peptide” as used herein refers to peptides, polypeptides, proteins and fragments of proteins, unless otherwise noted. The terms “protein” and “peptide” are used interchangeably herein when referring to a gene product and fragments thereof. Thus, exemplary peptides or proteins include gene products, naturally occurring proteins, homologs, orthologs, paralogs, fragments and other equivalents, variants, fragments, and analogs of the foregoing.

As used herein, the term “polysaccharide” refers to macromolecular carbohydrates whose molecule consists of a large number of monosaccharide molecules which are joined to one another by glycosidic linkage. The term polysaccharide is also intended to embrace an oligosaccharide. The polysaccharide can be homopolysaccharides or heteropolysaccharides. Whereas the homopolysaccharides contain only one kind of unit, the heteropolysaccharides consist of monomer units of different kinds.

The term “tissue” refers to a group or layer of similarly specialized cells which together perform certain special functions. The term “tissue-specific” refers to a source or defining characteristic of cells from a specific tissue.

The term “statistically significant” or “significantly” refers to statistical significance and generally means at least two standard deviation (2SD) away from a reference level. The term refers to statistical evidence that there is a difference. It is defined as the probability of making a decision to reject the null hypothesis when the null hypothesis is actually true.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

Other than in the operating examples, or where otherwise indicated, all numbers expressing quantities of ingredients or reaction conditions used herein should be understood as modified in all instances by the term “about.” The term “about” when used in connection with percentages can mean ±1%.

The singular terms “a,” “an,” and “the” include plural referents unless context clearly indicates otherwise. Similarly, the word “or” is intended to include “and” unless the context clearly indicates otherwise. Thus for example, references to “the method” includes one or more methods, and/or steps of the type described herein and/or which will become apparent to those persons skilled in the art upon reading this disclosure and so forth.

Further, unless otherwise required by context, singular terms shall include pluralities and plural terms shall include the singular.

Unless otherwise explained, all technical and scientific terms used herein have the same meaning as commonly understood by one of ordinary skill in the art to which this disclosure belongs.

It should be understood that this disclosure is not limited to the particular methodology, protocols, and reagents, etc., described herein and as such can vary. The terminology used herein is for the purpose of describing particular embodiments only, and is not intended to limit the scope of the present disclosure, which is defined solely by the claims.

All patents and other publications identified are expressly incorporated herein by reference for the purpose of describing and disclosing, for example, the methodologies described in such publications that might be used in connection with the present disclosure. These publications are provided solely for their disclosure prior to the filing date of the present application. Nothing in this regard should be construed as an admission that the inventors are not entitled to antedate such disclosure by virtue of prior disclosure or for any other reason. All statements as to the date or representation as to the contents of these documents is based on the information available to the applicants and does not constitute any admission as to the correctness of the dates or contents of these documents.

### BRIEF DESCRIPTION OF THE DRAWINGS

FIGS. 1A-1E show LPS treatment increases the expression of lnc-FAM164A1 in human macrophages, PBMCs and white blood cells in humanized Hu-CD34 NSG mice. Heatmap illustration of 11 lnc-RNAs (RP11-79H23.3 or lnc-FAM164A1), which were significantly increased by LPS stimulation in human PBMC-derived macrophages (4 different donors: A, B, C and D) (FIG. 1A). Human PBMC-derived macrophages were stimulated with 10 ng/mL LPS for 3h or 6h (FIG. 1B, P<0.05 LPS vs control; n=5-6 different donors). Human PBMC was isolated from citrated whole blood derived stimulated with 10 ng/mL LPS for 6 hours ex vivo (FIG. 1C, P<0.05 LPS vs control; n=3-4 different donors). Hu-CD34 NSG mice were stimulated with 5 mg/kg of LPS for 6 hours (FIG. 1D, P<0.05, NSG control vs NSG +LPS, n=4 mice per group). Relative fold change of the lnc-FAM164A1 expression was determined by RT-PCR (FIGS. 1B-1D). Representative image of RNAscope in situ staining of lnc-FAM164A1 on human PBMC-derived macrophages stimulated by LPS for 3 hours (Blue: DAPI; Red: lnc-FAM164A1; Green: human CD68) (FIG. 1E, white bar scale: 20 μM).

FIGS. 2A-2F show antisense or si-RNA oligonucleotides silencing of lnc-FAM164A1 reduces the expression of CCL2, IL-6 and TNF-α on human PBMC-derived macrophages. Human PBMC-derived macrophages were transfected with lnc-FAM164A1 antisense oligonucleotides (FAM) (FIGS. 2A-2F), or Axolab siRNA oligonucleotides (#5 and #8) (FIGS. 2G and 2H) or their nonspecific control oligonucleotides (NS) for 48h followed by 3-6 hours stimulation with LPS. Levels of CCL2 mRNA (FIGS. 2A and 2G), IL-6 mRNA (FIG. 2C), TNF-α mRNA (FIG. 2E) were detected by RT-PCR (P<0.05 NS-LPS vs FAM-LPS; n=4-6 different donors). CCL2 protein (FIG. 2B and 2H), IL-6 (FIG. 2D) and TNF-α protein (FIG. 2F) in culture medium were measured by ELISA (P<0.05 NS-LPS vs FAM-LPS; n=4-5 different donors).

FIGS. 3A-3G show enforced expression of lnc-FAM164A1 enhances the expression of pro-inflammatory TNF-α and CCL2 induced by LPS on human PBMC-derived macrophages. Human PBMC-derived macrophages were infected with LacZ control adenovirus (Ad-lacZ) or lnc-FAM164A1 expressing adenovirus (Ad-FAM) for 48h, and then stimulated with 10 ng/mL LPS for 2 hours. (FIG. 3A) Representative image of RNAscope in situ staining of lnc-FAM164A1 on adenovirus infected human PBMC-derived macrophages stimulated with or without LPS for 2 hours (Blue: DAPI; Red: lnc-FAM164A1). Expression of lnc-FAM164A1 RNA (FIGS. 3B and 3E), TNF-α mRNA and CCL2 mRNA (FIGS. 3C and 3F) was determined by RT-PCR; Protein levels of TNF-α and CCL2 in culture medium were measured by ELISA (FIGS. 3D and 3G). (P<0.05 Ad-LacZ vs Ad-FAM; n=5-7 independent experiments using different donors).

FIGS. 4A-4D show enforced expression of lnc-FAM164A1 promotes the activation of NF-κB signaling pathway in macrophages. (FIG. 4A) Enforced expression of lnc-FAM164A1 enhances the activation of NF-κB promoter assessed by NF-κB-luciferase reporter assay in HEK-293 cells (P<0.05 Vector vs Lnc-FAM164A1, n=9 samples). Enforced expression of lnc-FAM164A1 by adenovirus enhances the degradation of I-κB-α and the nuclear translocation of p65 induced by LPS for 15-60 minutes on THP-1-differentiated macrophages (FIG. 4B) and human PBMC-derived macrophages (FIGS. 4C and 4D). IκB-α and p65 protein in nuclear extracts were detected by western blots. β-actin and laminA/C were used as loading controls.

FIGS. 5A-5H show enforced expression of lnc-FAM164A1 enhances the induction of pro-inflammatory cytokines induced by LPS on mouse peritoneal macrophages. Mouse peritoneal were infected with LacZ control adenovirus (Ad-lacZ) or lnc-FAM164A1 expressing adenovirus (Ad-FAM) for 48h, and then stimulated with 10 ng/mL LPS for 3h. Expression oflnc-FAM164A1 RNA (FIG. 5A), mouse IL-1β mRNA (FIG. 5B), mouse TNF-α mRNA (FIG. 5C), and IL-6 mRNA (FIG. 5E) was determined by RT-PCR; Protein levels of TNF-α (FIG. 5D) and IL-6 (FIG. 5F) in culture medium were measured by ELISA. (P<0.05 LacZ vs FAM; n=6-9). Enforced expression of lnc-FAM164A1 enhances the induction of plasma TNF-α induced by LPS in mouse model of endotoxemia. C57BL/6J mice were infected with LacZ control adenovirus (Ad-lacZ) or lnc-FAM164A1 expressing adenovirus (Ad-FAM) for 3 days, and then stimulated with LPS for 3 hours. Expression of lnc-FAM164A1 RNA in the liver tissue (FIG. 5G) was determined by RT-PCR; Plasma levels of murine TNF-α protein (FIG. 5H) was measured by ELISA. (P<0.05 Ad-LacZ+LPS vs Ad-FAM+LPS; n=8-10 mice per group).

FIGS. 6A-6F show silencing of lnc-FAM164A1 in monocytes/macrophages decreases the expression of human CCL2 and IL-6 in a humanized mouse model of endotoxemia. Hu-CD34 NSG-SGM3 mice were administrated with lipid nanoparticle formulated si-RNA control oligos or si-lnc-FAM oligos, followed by LPS challenge for 3 hours. Expression of lnc-FAM164A1 RNA in purified monocytes (FIG. 6A) and human CD68, CCL2 and IL-6 mRNA in peritoneal cells (FIGS. 6B, 6D, and 6F) isolated from the endotoxemic mice was determined by RT-PCR. Plasma levels of human CCL2 and IL-6 protein were detected by ELISA (FIGS. 6C and 6E) (P<0.05, si-control +LPS vs si-lnc-FAM +LPS, n=6-7 mice per group).

FIGS. 7A-7D show mass spectrometry identified three proteins associated with lnc-FAM164A1. (FIG. 7A) Venn Diagram shows the number of proteins identified in three lnc-FAM164A1 RNA pulldown experiments (#1, #2, #3) using whole cell lysates from LPS-activated THP-1-differentiated macrophages. Those proteins have more than two fold change of the normalized AUC in the comparison of sense versus antisense (S/AS) or sense versus beads(S/B). Three proteins (ACLY, FLG2 and SFPQ) show up in both comparisons (S/AS and S/B). ACLY silencing decreases CCL2 mRNA expression induced by enforced expression of lnc-FAM164A1 in human PBMC-derived macrophages. (FIG. 7B) Disease associations of three lnc-FAM164A1-associated proteins (ACLY, FLG2 and SFPQ). (FIG. 7C) Input control of whole cell lysates from LPS-activated human PBMC-derived macrophages and the eluted proteins from Beads control (B), Antisense lnc-FAM164A1 (AS) and sense lnc-FAM164A1 (S) were detected by western blot using antibodies against human ACLY an hnRNPA1, respectively. Data shown were representative of three experiments. (FIG. 7D) ACLY silencing combined with enforced expression of lnc-FAM164A1 was performed on human PBMC-derived macrophages, followed by treatment with or without LPS for 2 hours. RNA expression of lnc-FAM164A1, ACLY and CCL2 were assessed by RT-PCR. (n=6-9 samples from three different experiments, p<0.05 by one-way ANOVA).

FIGS. 8A-8C show experimental scheme (FIG. 8A). Volcano plot of lnc-RNAs based on their relative expression levels (Log2 fold-change, LPS vs Control). 11 lnc-RNAs (Blue circles) have significant increase of expression induced by LPS treatment in human PBMC-derived macrophages (n=4, adjusted p-value FDR<0.05 and Fold-change≥2). Lnc-FAM164A1 highlighted in Red (FIG. 8B). Normalized intensity of each lnc-RNA in human lnc-RNA microarray analysis was shown as mean±SD (p<0.05, LPS vs control n=4 different donors) (FIG. 8C).

FIG. 9A-9C show human PBMC-derived macrophages were stimulated with 10 ng/mL human IL-6 and TNF-α for 3 hours. Relative fold change of the lnc-FAM164A1 expression was determined by RT-PCR (P<0.05 LPS vs control; n=3-4 different donors) (FIG. 9A). Hu-CD34 NSG mice were stimulated with LPS for 6 hours. Expression of lnc-FAM164A1 RNA in liver, spleen and lung isolated from the endotoxemic mice was determined by RT-PCR (P<0.05, Spleen vs Lung; n=4 mice per group) (FIG. 9B). (FIG. 9C) Ratio of lnc-FAM164A1 in cytoplasm and nucleus of PBMC-derived macrophages. Human PBMC-derived macrophages were stimulated with 10 ng/mL LPS for 3 hours. Nuclear and cytoplasmic RNA were separately purified. Lnc-FAM164A1 RNA, IL-1β and GAPDH mRNA expression were determined by RT-PCR and shown as the ratio of nuclear/cytoplasmic in unstimulated macrophages (Left Panel) and LPS-treated macrophages (Right Panel) (n=2 different donors).

FIGS. 10A and 18B. (FIG. 10A) Time course of lnc-FAM164A1 and cytokines expression in LPS-stimulated PBMC-derived macrophages. Human PBMC-derived macrophages were stimulated with 10 ng/mL LPS for 6, 12 and 24 hours. Lnc-FAM164A1 RNA, IL-1β, IL-6 and TNF-α mRNA expression were determined by RT-PCR and normalized by GAPDH expression. (Representative results of 2 different donors). (FIG. 10B) NF-κB inhibitor (Bay 11-7802) blocks the transcription of lnc-FAM164A1 and cytokines in LPS-stimulated PBMC-derived macrophages. Human PBMC-derived macrophages were pretreated with 3-30 μM Bay 11-7802 for 30 minutes before the stimulation with 10 ng/mL LPS for 6 hours. Lnc-FAM164A1 RNA, IL-1β, IL-6 and TNF-α mRNA expression were determined by RT-PCR and normalized by GAPDH expression (Representative data from 2 different donors).

FIGS. 11A-11E show antisense or si-RNA oligonucleotides silencing of lnc-FAM164A1 reduces the expression of CCL2, IL-6 and TNF-α on human PBMC-derived macrophages. Human PBMC-derived macrophages were transfected with lnc-FAM164A1 antisense oligonucleotides (FAM, FIGS. 11A and 11B), or Axolab siRNA oligonucleotides (#5 and #8, FIGS. 11C-11F) or their nonspecific control oligonucleotides (NS) for 48h followed by 3-6 hours stimulation with LPS. Levels of lnc-FAM164A1 RNA (FIGS. 11A and 11C), IL-10 mRNA (FIG. 11B), IL-6 (FIG. 11D) and TNF-α mRNA (FIG. 11E) were detected by RT-PCR (P<0.05 NS-LPS vs FAM-LPS; n=5-6 different donors). IL-6 protein (FIG. 11F) in culture medium were measured by ELISA (P<0.05 NS-LPS vs #5+LPS or #8+LPS; n=4 different donors).

FIGS. 12A-12L show effect of enforced expression of lnc-FAM164A1 on TNF-α, CCL2, IL-1β and IL-6 expression in THP-1-derived macrophages and human PBMC-derived macrophages. Human monocytic THP-1-derived macrophages (FIGS. 12A-12I) or human PBMC-derived macrophages (FIGS. 12J-12L) were infected with LacZ control adenovirus (Ad-lacZ) or lnc-FAM164A1 expressing adenovirus (Ad-FAM) for 48h, and then stimulated with 10 ng/mL LPS for 2-3 hours. Expression levels of lnc-FAM164A1 RNA (FIG. 12A) and TNF-α, CCL2, IL-1β and IL-6 mRNA (FIGS. 12B, 12D, 12F, 12H, 12J and 12K) were determined by RT-PCR. Protein levels of TNF-α, CCL2, IL-1β and IL-6 in culture medium were measured by ELISA (FIGS. 12C, 12E, 12G, 12I and 12L). (P<0.05 Ad-LacZ vs Ad-FAM; n=5-7 different experiments).

FIGS. 13A-13E. (FIG. 13A) Mouse peritoneal macrophages were infected with LacZ control adenovirus (Ad-lacZ) or lnc-FAM164A1 expressing adenovirus (Ad-FAM) for 48h, and then stimulated with 10 ng/mL LPS for 3h. (FIGS. 13B-13E) C57BL/6J mice were infected with LacZ control adenovirus (Ad-lacZ) or lnc-FAM164A1 expressing adenovirus (Ad-FAM) for 3 days, and then stimulated with LPS for 3 hours. Plasma levels of murine IL-6 (FIG. 13B) in the plasma were measured by ELISA. Expression levels of CCL2 mRNA in peritoneal macrophages (FIG. 13A) and IL-1β, IL-6 and TNF-α mRNA in liver were determined by RT-PCR (FIGS. 13C-13E). (P<0.05 Ad-LacZ vs Ad-FAM; n=9-10 mice per group).

FIGS. 14A-14F show numeration of Human CD45+WBC and monocytes in humanized Hu-CD34 NSG-SGM3 mice. Hu-CD34 NSG-SGM3 mice were administrated with lipid nanoparticle formulated si-RNA control oligos or si-lnc-FAM oligos, followed by LPS challenge for 3 hours. The percentage of human CD45+and mouse CD45+WBCs (FIGS. 14A, 14C and 14E) and human CD45+CD14+monocytes (FIGS. 14B, 14D and 14F) in the blood, spleen and bone marrow were detected by Flow Cytometry. (P<0.05, si-control+LPS vs si-lnc-FAM +LPS, n=6-7 mice per group).

FIGS. 15A-15F show mass Spectrometry identified three proteins associated with lnc-FAM164A1. (FIG. 15A) Relative normalized AUC of ACLY, FLG2 and SFPQ in Beads control (Beads), Antisense lnc-FAM164A1 (Antisense) and sense lnc-FAM164A1 (Sense) detected by MS (n=3, p<0.05 by t-test, Sense vs Beads; Sense vs Antisense). ACLY silencing decreases TNF-α expression induced by enforced expression of lnc-FAM164A1 in LPS-activated THP-1-differentiated macrophages. (FIG. 15B) Input control of whole cell lysates from LPS-activated THP-1-differentiated macrophages and the eluted proteins from Beads control (Beads), Antisense lnc-FAM164A1 (Antisense) and sense lnc-FAM164A1 (Sense) were detected by western blot using antibodies against human ACLY an hnRNPA1, respectively. Data shown were representative of three experiments. (FIGS. 15C-15F) ACLY silencing combined with enforced expression of lnc-FAM164A1 was performed on THP-1-differentiated macrophages, followed by treatment with or without LPS for 2 hours. RNA expression of ACLY, lnc-FAM164A1 and TNF-α were assessed by RT-PCR. TNF-α protein in culture medium was measured by ELISA (n=8-9 samples from three different experiments, p<0.05 by one-way ANOVA).

FIGS. 16A-16H show EVs released from activated RAW cells promote the activation of macrophages. EVs were isolated from culture medium of RAW cells infected with either Adeno-lacZ or Adeno-lnc-FAM164A1 virus. Resuspended EVs were incubated with RAW cells for 1 hour and followed by additional stimulation with LPS for 2 hours. Expression of lnc-FAM164A1 RNA and mouse cytokine mRNA in EVS (FIGS. 16A, 16C, 16E, and 16G, n=3-4 samples) and RAW cells (FIGS. 16B, 16D, 16F, and 16H) was detected by RT-PCR and normalized by the expression of GAPDH. (P<0.05, Ad-lacZ +LPS 2 hours vs Ad-FAM +LPS 2 hours, n=6 samples from two independent experiments).

FIG. 17 shows EVs isolated from culture medium of human PBMC-derived macrophages stimulated with LPS for 24 hours or from THP-1 cells infected with either Adeno-lacZ or Adeno-lnc-FAM164A1 virus for 24 hours and stimulated by LPS for 12 hours. Expression of lnc-FAM164A1 RNA in EVS was detected by RT-PCR and normalized by the expression of GAPDH (n=3 samples).

### DETAILED DESCRIPTION

The invention described herein is based on, in part, antisense oligonucleotide or siRNA silencing of lnc-FAM164A1 suppressed the LPS-induced expression of pro-inflammatory cytokines such as CCL2/MCP-1, IL-6 and TNF-α. Conversely, enforced expression of lnc-FAM164A1 in human and mouse primary macrophages enhanced the induction of pro-inflammatory cytokines by LPS. Luciferase reporter assay revealed lnc-FAM164A1 modulates the NF-κB signaling pathway. Enforced expression of lnc-FAM164A1 induced a rapid degradation of IKB-a and enhanced the nuclear translocation of p65 in LPS-stimulated human primary macrophages. Further, Lnc-FAM164A1 expression increased the plasma levels of TNF-α protein in the endotoxemic wild-type mice. In humanized mouse models of endotoxemia, LPS challenge increased the expression of lnc-FAM164A1 in blood leukocytes and spleen, and lnc-FAM164A1 silencing decreased the expression of human CCL2 and IL-6 in plasma and peritoneal cells.

Further described herein is work that shows RNA pulldown combined with mass spectrometry identified three proteins (ATP-citrate synthase (ACLY); Filaggrin-2 (Flg2); and Splicing factor, proline-and-glutamine-rich (SFPQ)) as being lnc-FAM164A1-associated proteins. Work described herein show that ACLY silencing decreases the expression of CCL2 and TNF-α induced by enforced expression of lnc-FAM164A1 in vitro. These data presented herein indicate that human lnc-FAM164A1 promotes pro-inflammatory activation of macrophages through its interaction with ACLY and NF-κB signaling and may participate in the pathogenesis of inflammatory diseases.

Long noncoding RNAs (lnc-RNAs, >200nt), non protein coding transcripts that play unsuspected roles in numerous biological processes and human diseases including embryonal development, tumor growth and metastasis, cardiometabolic disorders as well as inflammation. Several lnc-RNAs have been characterized as important regulators among the differentiation of immune cells, such as erythroid cells, T-lymphocytes, dendritic cells and monocytes/macrophages.

### Inflammatory Disease

An inflammatory disease or disorder is characterized by the activation of immune cells, for example macrophages (e.g., an activated macrophage), T cells, dendritic cells, B cells, or neutrophils. An inflammatory disease can be acute or chronic. In one embodiment, the macrophage activation macrophage is uncontrolled activation.

Macrophages are large phagocytes and a key player in innate immunity. Due to their ability to engulf pathogens, lipids, apoptotic cells, damaged tissue and to interplay with other immune cells, macrophages play an essential role in host defense and also contribute to the pathogenesis of various inflammatory diseases. Macrophages are remarkably plastic and can switch phenotype depending on the cellular stimulus in the tissue microenvironment. Alternatively, the balance of macrophage subpopulations can shift in response to molecular cues, determining the inflammatory state of normal or pathological tissues. Although human and murine macrophages express thousands of lnc-RNAs, only several lnc-RNAs have been characterized in modulating the activation of macrophages. In addition, several other mouse lnc-RNAs have been identified as modulators of inflammatory gene expression in macrophages and in mouse models of diabetes, atherosclerosis and endotoxemia.

Methods and compositions described herein are used to treat and/or prevent an inflammatory disease in a subject. Exemplary inflammatory diseases include, but are not limited to, endotoxemia, atherosclerotic vascular disease is coronary artery disease, stent restenosis, carotid metabolic disease, stroke, acute myocardial infarction, heart failure, peripheral arterial disease, limb ischemia, vein graft failure, AV fistula failure, Crohn's disease, ulcerative colitis, ileitis and enteritis; vaginitis; psoriasis and inflammatory dermatoses such as dermatitis, eczema, atopic dermatitis, allergic contact dermatitis, urticaria; vasculitis; spondyloarthropathies; scleroderma; respiratory allergic diseases such as asthma, allergic rhinitis, hypersensitivity lung diseases, arthritis (e.g. rheumatoid and psoriatic), eczema, psoriasis, osteoarthritis, multiple sclerosis, systemic lupus erythematosus, diabetes mellitus, glomerulonephritis, graft rejection (including allograft rejection and graft-v-host disease) or rejection of an engineered tissue, infectious diseases, myositis, inflammatory CNS disorders, stroke, closed-head injuries, neurodegenerative diseases, Alzheimer's disease, encephalitis, meningitis, osteoporosis, gout, hepatitis, hepatic veno-occlusive disease (VOD), hemorrhagic cystitis, nephritis, sepsis, sarcoidosis, conjunctivitis, otitis, chronic obstructive pulmonary disease, sinusitis, Bechet's syndrome, graft-versus-tumor effect, mucositis, appendicitis, ruptured appendix, peritonitis, or any other inflammatory disease known in the art. In one embodiment, the heart valve disease is aortic valve disease or mitral valve disease.

In another embodiment, the inflammatory disease occurs in response to a tissue (e.g., an organ, a blood vessel, or a blood valve) or cell transplantation. In another embodiment, the tissues can be artificial or engineered. As used herein, the term “transplantation” or “transplant” or “transplanted” refers to the transfer of viable cells or tissues from a donor or cell culture into a subject in need thereof. Transplantations can be done by a skilled physician in engraftment and infusion of cells and tissues (e.g. bone marrow transplantation). Non-limiting examples of tissues or cells that can be transplanted include cardiac, cardiac valve, vascular (vein or artery), bone, bone marrow, cartilage, tendons, ligaments, skeletal muscle, peripheral nervous tissue, brain, pancreas, liver, kidney, lung, bronchial smooth muscle, urinary, gastrointestinal tract, uterine, testicular, adipose, skin, hematopoietic stem cells, peripheral blood stem cells, and the like. Furthermore, the tissue or cells can be from a healthy donor subject that has donated cells or tissue that are viable and permit engraftment into a subject in need thereof. The in vitro-engineered tissues can be cultured prior to transplantation on a solid support, scaffold, or in tissue culture plastic dishes. There are many tissue-engineering techniques known in the art that can be used to make the tissue for transplantation into a subject in need thereof with a disease. Diseases and conditions the may require tissue or cell engraftment include but are not limited to cancer, anemias (e.g. aplastic anemia), blood diseases and disorders (e.g. sickle cell anemia), reconstructive surgeries of the bone, skeletal muscle, skin, and the like, spinal cord injuries, diabetes, cardiovascular diseases (e.g. valve diseases, stenosis, vascular reconstruction) or any other condition known in the art that requires the transplantation of a new tissue.

In another embodiment, the inflammatory disease can arise as a consequence of cancers such as acute myeloid leukemia (AML), chronic myeloid leukemia (CML), acute lymphoblastic leukemia (ALL), hodgkin lymphoma (HL) (relapsed, refractory), non-Hodgkin lymphoma (NHL) (relapsed, refractory), neuroblastoma, Ewing sarcoma, multiple myeloma, myelodysplastic syndromes, or gliomas. Additional non-limiting examples of cancer include carcinomas (e.g. adenocarcinoma, squamous cell carcinoma, adenosquamous carcinoma, anaplastic carcinoma, large and small cell carcinomas), sarcomas (e.g. osteosarcoma, Kaposi's sarcoma, lymphangiosarcoma, neurobibrosarcoma, dermatofibrosarcoma, etc), lymphomas, leukemias, blastomas (e.g. hepatoblastoma, medulloblastoma, nephroblastoma, pancreatoblastoma, pleuropulmonary blastoma, retinoblastoma, glioblastoma multiforme, gonadoblastoma), melanomas, seminomas, leiomyomas, and the like.

Treating and/or Preventing an Inflammatory Disease

One aspect provided herein is a method of treating or preventing an inflammatory disease in a subject comprising administering to a subject an effective amount of an agent that inhibits a long noncoding RNA(lnc-RNA) expressed in a macrophage.

In one embodiment, the lnc-RNA is AL078621.3; RP11-536K7.5; RP11-143M1.3; RP11-561O23.5; AC10980.2; P2RX7; LOC440896; D63785; RP11-58A12.2; RP11-79H23.3 (lnc-FAM164A1); or RP13-452N2.1.

Another aspect provided herein is a method of treating or preventing an inflammatory disease in a subject comprising administering to a subject an effective amount of an agent that inhibits lnc-FAM164A.

Another aspect provided herein is a method of treating or preventing an inflammatory disease in a subject comprising administering to a subject an effective amount of an agent that inhibits a protein bound to an lnc-RNA (e.g., lnc-FAM164A1). In one embodiment, the protein is ACLY, Flg2, or SFPQ. In one embodiment, the protein is ACLY.

In one embodiment of various aspects herein, the method further comprises, prior to administering, diagnosing a patient with having an inflammatory disease.

In another embodiment of various aspects herein, the method further comprises, prior to administering, receiving the results of an assay that diagnoses a patient as having an inflammatory disease. Assays that are useful in diagnosising a particular inflammatory disease are known in the art and can be performed by a skilled person. In one embodiment, the assay is performed at least 1 min, 1 hour, 1 day, 1 week, 1 month, 1 year, or more prior to administration of the agent.

Yet another aspect provided herein is a method of treating an inflammatory disease in a subject comprising (a) receiving results of an assay that measures a level of a lnc-RNA expressed in a macrophage in a biological sample; (b) comparing the level of the lnc-RNA to a reference level; (c) identifying a subject as having an inflammatory disease if the level of the lnc-RNA is significantly increased as compared to the reference level; and (d) administering to the subject having an inflammatory disease an agent that inhibits the lnc-RNA, or a composition comprising an agent that inhibits the lnc-RNA.

Another aspect provided herein is a method of treating an inflammatory disease in a subject comprising (a) measuring a level of a lnc-RNA expressed in a macrophage in a biological sample; (b) comparing the level of the lnc-RNA to a reference level; (c) identifying a subject as having an inflammatory disease if the level of the lnc-RNA is significantly increased as compared to the reference level; and (d) administering to the subject having an inflammatory disease an agent that inhibits the lnc-RNA, or a composition comprising an agent that inhibits the lnc-RNA. In one embodiment of any aspect, the method further comprises, prior to step a), obtaining a biological sample from the subject.

In one embodiment, the lnc-RNA is inhibited in a target cell. In one embodiment, the target cell is a mammalian cell, for example, a human cell. In on embodiment, a target cell can be a xemplary target cells include, but are not limited to a blood cell, a macrophage, an activated macrophage, a T cell, a dendritic cell, a B cell, a natural killer cell, or a neutrophil. Additional non-limiting examples of a target cell can further include stem cells, cancer cells, vascular endothelium, cardiac cells, cardiac valve cells, pancreatic cells, adipose cells, neurons, hepatic cells, kidney cells, osteoclasts, osteoblasts, fibroblasts, and the like. One skilled in the art can identify a cell type, for example, based on its gene expression pattern via immunofluorescence, or sequence-based anaylsis (e.g., genome sequencing). The gene expression pattern for the exemplary cell types are known in the art.

A macrophage is considered to be “activated” when the macrophage is (1) able to phagocytose or kill another cell, microbe, or fragment thereof, and/or (2) expresses or releases cytokines such as IL-1β, TNF-α, IL-6, IL-8, or any other macrophage molecules (e.g proteases, neutrophil chemotatic factors; reactive oxygen species such as nitric oxide and superoxide, eicosanoids or growth factors) known in the art. The products of activated macrophages result in tissue destruction and inflammation.

In one embodiment, inhibiting results in the reduction of the level or activity of the agent's target (e.g., a lnc-RNA expressed on a macrophage, lnc-FAM164A1, or a protein bound to a lnc-RNA, e.g., ACLY).

In one embodiment, inhibiting a lnc-RNA expressed on a macrophage inhibits the level or activity of the lnc-RNA expressed on the macrophage. The lnc-RNA expressed on a macrophage activity can be any currently known activity, or yet to be discovered activity lnc-RNA expressed on a macrophage. In one embodiment, the lnc-RNA expressed on a macrophage level or activity is inhibited by at least 5%, 10%, 15%, 20%, 25%, 30%, 35%, 40%, 45%, 50%, 55%, 60%, 65%, 70%, 75%, 80%, 85%, 90%, 95%, 99%, or more as compared to an appropriate control. As used herein, an appropriate control would be the lnc-RNA expressed on a macrophage level or activity in an otherwise identical sample that is not contacted by an agent or composition described herein, or is the level or activity of the lnc-RNA expressed on a macrophage in a subject prior to administration of an agent or composition. One skilled in the art can determine if the activity of the lnc-RNA expressed on a macrophage is reduced following administration of an agent or composition described herein using functional readouts of lnc-RNA expressed on a macrophage activity, for example, phagocytosis, and cytokine expression by real time-polymerase chain reacation (RT-PCR), immunohistochemistry, or flow cyometry. One skilled in the art can determine if the level of the lnc-RNA expressed on a macrophage is reduced following administration of an agent or composition described herein using PCR-based assays (e.g., quantitative PCR) to directly measure the level of the lnc-RNA.

In one embodiment, inhibiting lnc-FAM164A1 inhibits the level or activity of lnc-FAM164A1. The lnc-FAM164A1 activity can be any currently known activity, or yet to be discovered activity of lnc-FAM164A1. In one embodiment, lnc-FAM164A1 level or activity is inhibited by at least 5%, 10%, 15%, 20%, 25%, 30%, 35%, 40%, 45%, 50%, 55%, 60%, 65%, 70%, 75%, 80%, 85%, 90%, 95%, 99%, or more as compared to an appropriate control. As used herein, an appropriate control would be lnc-FAM164A1 activity or level in an otherwise identical sample that is not contacted by an agent or composition described herein, or is the level or activity of lnc-FAM164A1 in a subject prior to administration of an agent or composition. One skilled in the art can determine if the activity of lnc-FAM164A1 is reduced following administration of an agent or composition described herein using functional readouts of lnc-FAM164A1 activity, for example, phagocytosis, and cytokine expression by real time-polymerase chain reacation (RT-PCR), immunohistochemistry, or flow cyometry. Examplary cytokines that exhibit a modulation in their expression include, but are not limited to, IL-1β, IL-6, or TNF-α. One skilled in the art can determine if the level of the lnc-FAM164A1 is reduced following administration of an agent or composition described herein using PCR-based assays (e.g., quantitative PCR) to directly measure the level of the lnc-RNA.

In one embodiment, inhibition of lnc-FAM164A1 results in decreased expression of CCL2 and IL-6 in a plasma cell and/or a peritoneal cell. One skilled in the art can determine if the level of CCL2 and IL-6 is reduced in a plasma cell and/or a peritoneal cell following administration of an agent or composition described herein using PCR-based assays (e.g., quantitative PCR) or westernblotting to directly measure the mRNA or protein level of CCL2 and IL-6 as compared to an appropriate control, respectively. As used herein, an appropriate control refers to the level of of CCL2 and IL-6 in a plasma cell and/or a peritoneal cell that is not contacted by an agent or compostion described herein, or the level of CCL2 and IL-6 in a plasma cell and/or a peritoneal cell prior to administration of the agent or composition. One skilled in the art can identify or isolate a plasma cell and/or a peritoneal cell using standard techniques, for example, flow cytometery that sorts the cell for particular cell surface markers specific for a plasma cell and/or a peritoneal cell, for example, a plasma cell can be identified by expression of CD138, CD78 and IL-6, and a peritoneal cell can be identified by expression of B220, CD80, and CD43.

In one embodiment, inhibition of lnc-FAM164A1 results in decreased NFkB signaling. One skilled in the art can assess NFkB signaling using functional readouts that assess the activation of NFkB targets, for example, assessing p65 nuclear import, which occurs upson NFkB signal pathway activation.

In one embodiment, inhibiting a protein bound to a lnc-RNA inhibits the level or activity of the protein. The activity of the protein can be any currently known activity, or yet to be discovered activity. In one embodiment, the level or activity of the protein is inhibited by at least 5%, 10%, 15%, 20%, 25%, 30%, 35%, 40%, 45%, 50%, 55%, 60%, 65%, 70%, 75%, 80%, 85%, 90%, 95%, 99%, or more as compared to an appropriate control. As used herein, an appropriate control would be activity or level of the protein in an otherwise identical sample that is not contacted by an agent or composition described herein, or is the level or activity of the protein in a subject prior to administration of an agent or composition. One skilled in the art can determine if the activity of the protein is reduced following administration of an agent or composition described herein using functional readouts of the protein's activity. One skilled in the art can determine if the level of the protein is reduced following administration of an agent or composition described herein using PCR-based assays (e.g., quantitative PCR) or westerblotting to directly measure the mRNA or protein level of the protein, respectively.

In one embodiment, inhibiting a protein bound to a lnc-RNA inhibits binding of the protein to a lnc-RNA, for example, lnc-FAM164A1. In one embodiment, binding of the protein to a lnc-RNA is inhibited by at least 5%, 10%, 15%, 20%, 25%, 30%, 35%, 40%, 45%, 50%, 55%, 60%, 65%, 70%, 75%, 80%, 85%, 90%, 95%, 99%, or more as compared to an appropriate control. As used herein, an appropriate control would be binding of the protein to a lnc-RNA in an otherwise identical sample that is not contacted by an agent or composition described herein, or is the binding of the protein to a lnc-RNA in a subject prior to administration of an agent or composition. One skilled in the art can determine if the binding of the protein to lnc-RNA is reduced following administration of an agent or composition described herein using functional readouts of ACLY binding, for example, co-immunoprecipation of ACLY and lnc-RNA.

In one embodiment, inhibiting ACLY inhibits the level or activity of ACLY. The ACLY activity can be any currently known activity, or yet to be discovered activity of ACLY. In one embodiment, ACLY level or activity is inhibited by at least 5%, 10%, 15%, 20%, 25%, 30%, 35%, 40%, 45%, 50%, 55%, 60%, 65%, 70%, 75%, 80%, 85%, 90%, 95%, 99%, or more as compared to an appropriate control. As used herein, an appropriate control would be ACLY activity or level in an otherwise identical sample that is not contacted by an agent or composition described herein, or is the level or activity of ACLY in a subject prior to administration of an agent or composition. One skilled in the art can determine if the activity of ACLY is reduced following administration of an agent or composition described herein using functional readouts of ACLY activity, for example, catalyzing the conversion of citrate and CoA into acetyl-CoA and oxaloacetate, and hydrolysis of ATP. One skilled in the art can determine if the level of the ACLY is reduced following administration of an agent or composition described herein using PCR-based assays (e.g., quantitative PCR) or westerblotting to directly measure the mRNA or protein level of ACLY, respectively.

In one embodiment, inhibiting ACLY binding of ACLY to a lnc-RNA, for example, lnc-FAM164A1. In one embodiment, ACLY binding to a lnc-RNA is inhibited by at least 5%, 10%, 15%, 20%, 25%, 30%, 35%, 40%, 45%, 50%, 55%, 60%, 65%, 70%, 75%, 80%, 85%, 90%, 95%, 99%, or more as compared to an appropriate control. As used herein, an appropriate control would be ACLY binding to a lnc-RNA in an otherwise identical sample that is not contacted by an agent or composition described herein, or is the binding of ACLY to a lnc-RNA in a subject prior to administration of an agent or composition. One skilled in the art can determine if the binding of ACLY to lnc-RNA is reduced following administration of an agent or composition described herein using functional readouts of ACLY binding, for example, co-immunoprecipation of ACLY and lnc-RNA.

In one embodiment, inhibition of ACLY results in decreased expression of CCL2 and TNF-α in the subject. One skilled in the art can determine if the level of CCL2 and TNF-α following administration of an agent or composition described herein using PCR-based assays (e.g., quantitative PCR) or western blotting to directly measure the mRNA or protein level of CCL2 and TNF-α as compared to an appropriate control, respectively. As used herein, an appropriate control refers to the level of CCL2 and TNF-α in a biological sample that is not contacted by an agent or compostion described herein, or the level of CCL2 and TNF-α in a plasma cell and/or a peritoneal cell prior to administration of the agent or composition.

In one embodiment, the subject has previously been diagnosed with having an inflammatory disease. In one embodiment, the subject has not previously been diagnosed with having an inflammatory disease. A subject can have previously received a treatment or therapeutic for a given inflammatory disease.

In one embodiment, the subject exhibits at least one risk factor of developing an inflammatory disease. Exemplary risks factors for an inflammatory disease are known in the art, and can be properly assessed by a skilled practitioner.

Sepsis is a potentially life-threatening complication of an infection. Sepsis occurs when chemicals released into the bloodstream to fight the infection trigger inflammatory responses throughout the body. This inflammation can trigger a cascade of changes that can damage multiple organ systems, causing them to fail. Another aspect provides a method of identifying a subject having sepsis, the method comprising (a) measuring a level of a lnc-FAM164A1 in a biological sample; (b) comparing the level of the lnc-FAM164A1 to a reference level; (c) identifying a subject as having sepsis if the level of the lnc-RNA is significantly increased as compared to the reference level; and (d) administering to the subject having sepsis a therapeutic to treat sepsis. In one embodiment, the method further comprises, prior to step (a), obtaining a biological sample from the subject. In one embodiment, the biological sample is a blood sample. Agents

In various embodiments, the agent that is administered to a subject to treat or prevent an inflammatory disease inhibits an lnc-RNA expressed in a macrophage, lnc-FAM164A1, or a protein bound to a lnc-RNA, for example, ACLY.

In one embodiment, a subject is administered an agent that inhibits a lnc-RNA expressed in a macrophage to treat of prevent an inflammatory disease. In one embodiment, the lnc-RNA is AL078621.3; RP11-536K7.5; RP11-143M1.3; RP11-561O23.5; AC10980.2; P2RX7; LOC440896; D63785; RP11-58A12.2; RP11-79H23.3 (lnc-FAM164A1); or RP13-452N2.1. An agent described herein is considered effective for inhibiting a lnc-RNA expressed in a macrophage if, for example, upon administration, it inhibits the presence, amount, activity and/or level of a lnc-RNA expressed in a macrophage.

In one embodiment, a subject is administered an agent that inhibits lnc-FAM164A1 to treat or prevent an inflammatory disease. An agent described herein is considered effective for inhibiting lnc-FAM164A1 if, for example, upon administration, it inhibits the presence, amount, activity and/or level of lnc-FAM164A1 in the cell.

In yet another embodiment, a subject is administered an agent that inhibits a protein bound to a lnc-RNA to treat or prevent an inflammatory disease. An agent described herein is considered effective for protein bound to a lnc-RNA if, for example, upon administration, it inhibits the presence, amount, binding of, activity and/or level of the protein in the cell.

In yet another embodiment, a subject is administered an agent that inhibits ACLY to treat or prevent an inflammatory disease. An agent described herein is considered effective for inhibiting ACLY if, for example, upon administration, it inhibits the presence, amount, binding of activity and/or level of ACLY in the cell.

In one embodiment, the agent is a small molecule, an antibody, a peptide, a genome editing system, an antisense oligonucleotide, and an RNA interference (RNAi), an antisense RNA, an RNA decoy molecule, an RNAaptamer, and an inhibitory polypeptide. In one embodiment, the RNAi is a microRNA, an siRNA, or a shRNA

An agent can inhibit e.g., the transcription of lnc-RNA expressed in a macrophage or a protein bound to a lnc-RNA, for example, ACLY in a cell. An agent can inhibit the activity or alter the activity (e.g., such that the activity no longer occurs, no longer occurs properly (e.g., as compared to wild-type lnc-RNA or lnc-FAM164A1 activity), or occurs at a reduced rate of in the cell (e.g., lnc-RNA expression in a macrophage or ACLY expression).

The agent may function directly in the form in which it is administered. Alternatively, the agent can be modified or utilized intracellularly to produce something which inhibits, for example, lnc-RNA in a macrophage or a protein bound to a lnc-RNA, for example, ACLY, such as introduction of a nucleic acid sequence into the cell and its transcription resulting in the production of the nucleic acid and/or protein inhibitor of lnc-RNA in a macrophage or ACLY within the cell. In some embodiments, the agent is any chemical, entity or moiety, including without limitation synthetic and naturally-occurring non-proteinaceous entities. In certain embodiments, the agent is a small molecule having a chemical moiety. For example, chemical moieties included unsubstituted or substituted alkyl, aromatic, or heterocyclyl moieties including macrolides, leptomycins and related natural products or analogues thereof. Agents can be known to have a desired activity and/or property, or can be identified from a library of diverse compounds.

In various embodiments, the agent is a small molecule that inhibits lnc-RNA expressed in a macrophage or a protein bound to a lnc-RNA, for example, ACLY. As used herein, the term “small molecule” refers to a chemical agent which can include, but is not limited to, a peptide, a peptidomimetic, an amino acid, an amino acid analog, a polynucleotide, a polynucleotide analog, an aptamer, a nucleotide, a nucleotide analog, an organic or inorganic compound (e.g., including heterorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. Methods for screening small molecules are known in the art and can be used to identify a small molecule that is efficient at, for example, inhibiting lnc-RNA (e.g., expressed in a macrophage) or ACLY activity or levels, or ACLY binding to lnc-RNA, given the desired target (e.g., lnc-FAM164A1).

In one embodiment, the small molecule inhibitor of ACLY is SB 204990, BMS 303141, and MEDICA 16 (e.g., available from Tocris; Minneapolis, Minn.), and Bempedoic acid. Inhibitors of ACLY are further described in, e.g., Granchi, C. European Journal of Medicial Chemistry. (2018) 157: 1276-1291, which is incorporated herein by reference in its entirety.

In one embodiment, the small molecule that inhibits lnc-FAM164A1 is a NFkB signaling pathway inhibitor. In one embodiment, the small molecule that inhibits lnc-FAM164A1 is the NFkB signaling pathway inhibitor Bay 11-7802.

In one embodiment, the agent is an antibody or antigen-binding fragment thereof, or an antibody reagent that is specific for lnc-RNA expressed in a macrophage or a protein bound to a lnc-RNA, for example, ACLY. As used herein, the term “antibody reagent” refers to a polypeptide that includes at least one immunoglobulin variable domain or immunoglobulin variable domain sequence and which specifically binds a given antigen. An antibody reagent can comprise an antibody or a polypeptide comprising an antigen-binding domain of an antibody. In some embodiments of any of the aspects, an antibody reagent can comprise a monoclonal antibody or a polypeptide comprising an antigen-binding domain of a monoclonal antibody. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody reagent” encompasses antigen-binding fragments of antibodies (e.g., single chain antibodies, Fab and sFab fragments, F(ab')2, Fd fragments, Fv fragments, scFv, CDRs, and domain antibody (dAb) fragments (see, e.g. de Wildt et al., Eur J. Immunol. 1996; 26(3):629-39; which is incorporated by reference herein in its entirety)) as well as complete antibodies. An antibody can have the structural features of IgA, IgG, IgE, IgD, or IgM (as well as subtypes and combinations thereof). Antibodies can be from any source, including mouse, rabbit, pig, rat, and primate (human and non-human primate) and primatized antibodies. Antibodies also include midibodies, nanobodies, humanized antibodies, chimeric antibodies, and the like.

In one embodiment, the agent is a humanized, monoclonal antibody or antigen-binding fragment thereof, or an antibody reagent. As used herein, “humanized” refers to antibodies from non-human species (e.g., mouse, rat, sheep, etc.) whose protein sequence has been modified such that it increases the similarities to antibody variants produce naturally in humans. In one embodiment, the humanized antibody is a humanized monoclonal antibody. In one embodiment, the humanized antibody is a humanized polyclonal antibody. In one embodiment, the humanized antibody is for therapeutic use.

In one embodiment, the anti-lnc-RNA antibody that inhibits an lnc-RNA expressed on a macrophage is any known anti-lnc-RNA antibodies in the art, or any anti-lnc-FAM164A1 antibodies that are yet to be discovered. In one embodiment, the anti-lnc-FAM164A1 antibody is a humanized anti-lnc-FAM164A1 antibody derived from any known, or yet to be discovered, non-human anti-lnc-FAM164A1 antibody.

In one embodiment, the anti-lnc-FAM164A1 antibody is any known anti-lnc-FAM164A1 antibodies in the art, or any anti-lnc-FAM164A1 antibodies that are yet to be discovered. In one embodiment, the anti-lnc-FAM164A1 antibody is a humanized anti-lnc-FAM164A1 antibody derived from any known, or yet to be discovered, non-human anti-lnc-FAM164A1 antibody.

In another embodiment, the anti-LNC-FAM164A1 antibody or antibody reagent binds to a nucleotide sequence that comprises the sequence of SEQ ID NO: 1; or binds to a nucleotide sequence that comprises a sequence with at least 80%, at least 85%, at least 90%, at least 95%, at least 96%, at least 97%, at least 98%, at least 99% or greater sequence identity to the sequence of SEQ ID NO: 1. In one embodiment, the anti-lnc-FAM164A1 antibody or antibody reagent binds to a nucleotide sequence that comprises the entire sequence of SEQ ID NO: 1. In another embodiment, the antibody or antibody reagent binds to a nucleotide sequence that comprises a fragment of the sequence of SEQ ID NO: 1, wherein the fragment is sufficient to bind its target, e.g., lnc-FAM164A1, and result in the inhibition of lnc-FAM164A1 level and/or activity.

SEQ ID NO: 1 is the nucleotide sequence of lnc-FAM64A1,

(SEQ ID NO: 1)
TCTAAATCCCATTTCTATTCTATTATTATTACTACTACAAAAATAGTAAT
AAAAACAAACATTTAAATAATATCTAATAAATACCAACTATTCTTCTAAG
AACTTGACATGTATAAACTCATTTAAGCTTCAACTCTGTGAAATAGGGTA
TCAGTTATGTATTGCCAAAATAACGCTATATAGCAAACTACCCCAAAACA
TAAAACTGTAAAAACATAATTACAACAATAGACCTTTTTATCCTTTCTCA
CAAGCCACTGTTTTTGCTGATCTGGGCCAAGTTCAAAAGACATTGACTTG
GCCCACTCACCTGTGGACAGTCAGCTGCCTTGTTGGATGGGTGCTGGGAG
TATAAGATGGCCTCAGTTAGGACTGCTTGGCTGTTCTCTAGAAGTTCTCT
CATTTTCACAATGCTAGCCTGGACTTATTTCCATTGCCATAGGAGGGTTC
TAACAGAGAGAAAGAGAGCGGAAGCAGGCAATGCTTCTTAAAGCCTAGGT
TTGGAATGCACACTGTTACTTCCCACGTATGGTATTGGACAGAGCAAGTG
AAAACACCTAGTGCCAAGAGTATGCATCCAGGAAGGGGTAAAGAACAGGA
CTAGTTTTGCCATTTATTAAGGTATTATTATTAATCCTTATTTTAGAAGA
AACTGATATATAGAGGAGTTAAGCAACTTGCTCAAGGTCGTGGACTAATG
AAACAATGGCACTAGTATTTAAATACTGTCAGTCTGGTTTCATTCTGTGC
TGTTAACTTCTACATGGTGCTGTCAACTTCTACATGGTGCTGTCTCTCAG
CTGAAGGAACGATAGAGGAGGAAAGGCAAAAGGCAACAATGGGCTTTGAA
GTAACTCTACTGAGATAATTAAAAGACCACATAAAAGAAGGAAAACGTAT
CTTACTTCAGGAATTCAACACTAAGAATCCTACTCCCTTCGTGAAATCAG
AAAAGGTTTGCATGAACCCTGTGGGCCTTTGAAAGAGAGAAGGCATACTG
TTGATGGTCACCAACTTACTGAAGCCTGCAGCTTGCAGGAACCCATCTCA
TCAGAGTAACTGAAAGAGAACACAGTATCCAAAAACAAAAACAACAAAAG
TCACGTGCTGGGATAAGTTCTTGATAGTGTGAATAGTCAGAAAAAAATGT
TCTGAAACTTCTTGTCCCAGCTGCGTTATTAACTGTGAATTTCATCATAT
GGTTGATTAAGTTGCTTTATAAGACTAAATTTTTATAAGATGTGTACATA
GGAATATGACTTCCCTCTCCTAAGTTAACATAACAGGTATTCCCAAATGT
GTTGTGAAGTCGACATTCTTGTTCTAATTTATAGTTGTATTGCTCCCTGC
AATAGCATCTTCTTTCATTTCCCAGTCTCCAGTTTTTCCATTTTGTTTAC
AGTAGCCAGAGAAATCTATCAAGATGCACACTTGATCACAGCATCCTTCG
GTAAATCACCATTATCTTCATACTGACAAAGGTCAGTCTTTAAGGACCTT
TATCAGGTGTCCTCTCTTTAGTTCCCCAACCTATTTCTTACCATCCTTTA
TCCCCCAGCTAAATAACTTGGAGAATTCCAAGTGCGTCATCCACTGTATT
AGTCTGTTCTCACATCGCTAACAAAGACATACCTGAGACTGGGGAATTTA
TAAAGGAAAGAGGTTTAATGGACTCACAGTTCCGCAGGGCTAGGGAGGCT
TCACAATCATGGCAGAAGACAAAGCAAGAGCAAAGGGAAGTCTTACATGG
CAGCAGGCAAGAGAGCTTGTGCAGGGGGAACTCCTGTTTATAAAACCATC
AGATCTTGGGAGATGTGTTCAATACGATGAGAACAGTGTGGGGGAAACCA
CCCCCATGATGCCATTATCTCCACTTGTCTCTGTCCTTGACACATGGGAA
TTATTATAATTCTAGGTGAGATTTGGGTGGGGACACAGAGCCAAACAATA
TCATCCACCTTCATGTCTCTTTCTCTTTACTCACCTGCTTTCTTGACAAG
AAATATCTTCATCAGCCCTTCCTTCCTTCCCTCCCTCCCTCCTTTCTTTC
TTTCTTGCTTGCTTGCTTTTCCTTCCTTTTTCTTCTTTATTTCCTTTGCC
AATTTGAGAGATTGGCAAGGAGAGTAATGCTGGAATTGGAGACACATATA
ACTAATACCAGCCAAAATAATTGTGAGCAGAAGTGATATGCCTTCTGAGC
CAAGGCAGAGAAAAGATTATATGTGATTCTCAGACCTCTTCTCATCCTCA
TTGGCTAAGAAGGGCATAGGTCCCAGATGGAACAGCTATACGTTAATGGG
GCCTTCCTCCATTTGGATCCCTAAGTAACTGAAATGGAGCCCCTCTCCAA
CCTGTGATAAAATTGTGCTACAAAGGAGAACTCAATTTTCTTGTGCTAAA
ATACTGAGATTTGGAAATTAATGTGTGCTGCATTATAACCTAGGTTATTA
TAAAGATATTATTGAAAATTACTTTTAATATTCATTTTTGTCATTATTGG
TGAATTTGAAGTTTCATATAATTATTGAACACTTATATGTCATTTATATA
TTTATTATACTTAGTTCTTTGGCAAAATGCTGTTTATGTCCTTTGCCTAT
TTTTCTACTAGACTGTTTATGTTGATTTATAAAACATATTTATATATTAG
GCTACAGTAACCCTTTCATGTCATTGGTACTGTTTCCAATTTATTAACTG
CCTTTTATTATCACTTTATGATGTATTTTATGTCCAAAGGTTTAAAAATT
ATATACTGAGTAATCGATAATTATTTAAAAATTTTGTTCTTATTTTATGA
GAAAGGCCTTCTAAAATCTGATAGGTTGGTTTTTCTCTGGATATACTATT
TTCTATTTTTTAAAACATGAAATTCTATACTCCATTATGATTTATGTTGT
TGCATGCTATCTGGTCGGAATATAGCTTTGTAATGAATACATTCGTCTTA
AAACTGACTTGGCTTTTTCTCCTAAATTAAAAAGACAACTCTGA

In one embodiment, the anti-ACLY antibody is any known anti-ACLY antibodies in the art, or any anti-ACLY antibodies that are yet to be discovered. In one embodiment, the anti-ACLY antibody is a humanized anti-ACLY antibody derived from any known, or yet to be discovered, non-human anti-ACLY antibody. Anti-ACLY monoclonal antibody clones are known in the art, for example, anti-ACLY antibody clone OTI3G8, 4H6L2, 5F8D11, and 4D11. The structure, sequences, and functions for these anti-ACLY monoclonal antibody clones are known in the art.

In another embodiment, the anti-ACLY antibody or antibody reagent binds to an amino acid sequence that comprises the sequence of SEQ ID NO:2 ; or binds to an amino acid sequence that comprises a sequence with at least 80%, at least 85%, at least 90%, at least 95%, at least 96%, at least 97%, at least 98%, at least 99% or greater sequence identity to the sequence of SEQ ID NO: 2. In one embodiment, the anti-ACLY antibody or antibody reagent binds to an amino acid sequence that comprises the entire sequence of SEQ ID NO: 2. In another embodiment, the antibody or antibody reagent binds to an amino acid sequence that comprises a fragment of the sequence of SEQ ID NO: 2, wherein the fragment is sufficient to bind its target, e.g., ACLY, and result in the inhibition of ACLY level and/or activity.

SEQ ID NO: 2 is an amino acid seauence that encodes ACLY.

(SEQ ID NO: 2)
MSAKAISEQTGKELLYKFICTISAIQNRFKYARVTPDTDWARLLQDHPWL
LSQNLVVKPDQLIKRRGKLGLVGVNLTLDGVKSWLKPRLGQEATVGKATG
FLKNFLIEPFVPHSQAEEFYVCIYATREGDYVLFHHEGGVDVGDVDAKAQ
KLLVGVDEKLNPEDIKKHLLVHAPEDKKEILASFISGLFNFYEDLYFTYL
EINPLVVIKDGVYVLDLAAKVDATADYICKVKWGDIEFPPPFGREAYPEE
AYIADLDAKSGASLKLTLLNPKGRIWTMVAGGGASVVYSDTICDLGGVNE
LANYGEYSGAPSEQQTYDYAKTILSLMTREKHPDGKILIIGGSIANFTNV
AATFKGIVRAIRDYQGPLKEHEVTIFVRRGGPNYQEGLRVMGEVGKTTGI
PIHVFGTETHMTAIVGMALGHRPIPNQPPTAAHTANFLLNASGSTSTPAP
SRTASFSESRADEVAPAKKAKPAMPQDSVPSPRSLQGKSTTLFSRHTKAI
VWGMQTRAVQGMLDFDYVCSRDEPSVAAMVYPFTGDHKQKFYWGHKEILI
PVFKNMADAMRKHPEVDVLINFASLRSAYDSTMETMNYAQIRTIAIIAEG
IPEALTRKLIKKADQKGVTIIGPATVGGIKPGCFKIGNTGGMLDNILASK
LYRPGSVAYVSRSGGMSNELNNIISRTTDGVYEGVAIGGDRYPGSTFMDH
VLRYQDTPGVKMIVVLGEIGGTEEYKICRGIKEGRLTKPIVCWCIGTCAT
MFSSEVQFGHAGACANQASETAVAKNQALKEAGVFVPRSFDELGEIIQSV
YEDLVANGVIVPAQEVPPPTVPMDYSWARELGLIRKPASFMTSICDERGQ
ELIYAGMPITEVFKEEMGIGGVLGLLWFQKRLPKYSCQFIEMCLMVTADH
GPAVSGAHNTIICARAGKDLVSSLTSGLLTIGDRFGGALDAAAKMFSKAF
DSGIIPMEFVNKMKKEGKLIMGIGHRVKSINNPDMRVQILKDYVRQHFPA
TPLLDYALEVEKITTSKKPNLILNVDGLIGVAFVDMLRNCGSFTREEADE
YIDIGALNGIFVLGRSMGFIGHYLDQKRLKQGLYRHPWDDISYVLPEHMS
M

In one embodiment, the agent is an antisense oligonucleotide. As used herein, an “antisense oligonucleotide” refers to a synthesized nucleic acid sequence that is complementary to a DNA or mRNA sequence, such as that of a microRNA. Antisense oligonucleotides are typically designed to block expression of a DNA or RNA target by binding to the target and halting expression at the level of transcription, translation, or splicing. Antisense oligonucleotides of the present invention are complementary nucleic acid sequences designed to hybridize under cellular conditions to a gene, e.g., a lnc-RNA expressed in a macrophage, or a protein bound to a lnc-RNA, for example, ACLY. Thus, oligonucleotides are chosen that are sufficiently complementary to the target, i.e., that hybridize sufficiently well and with sufficient specificity in the context of the cellular environment, to give the desired effect.

In one embodiment, an antisense oligonucleotide that inhibits lnc-FAM164A1 may comprise at least 5, at least 10, at least 20, at least 30, at least 40, at least 50, at least 60, at least 70, at least 80, at least 90, at least 100, at least 200, at least 300, at least 400, at least 500, at least 600, at least 700, at least 800, at least 900, at least 1000, at least 2000, or more bases complementary to a portion of the coding sequence of the human lnc-FAM164A1 gene. In one embodiment, the coding sequence of the lnc-FAM164A1 gene is the sequence of SEQ ID NO: 1.

In one embodiment, an antisense oligonucleotide that inhibits ACLY may comprise at least 5, at least 10, at least 20, at least 30, at least 40, at least 50, at least 60, at least 70, at least 80, at least 90, at least 100, at least 200, at least 300, at least 400, at least 500, at least 600, at least 700, at least 800, at least 900, at least 1000, at least 2000, or more bases complementary to a portion of the coding sequence of the human ACLY gene. In one embodiment, the coding sequence of the ACLY gene is the sequence of SEQ ID NO: 3.

SEQ ID NO: 3 is the nucleotide sequence of ACLY, e.g., for isoform 1.

(SEQ ID NO: 3)

| | |
|---|---|
| | atgtcgg ccaaggcaat |
| 181 | ttcagagcag acgggcaaag aactccttta caagttcatc |
| | tgtaccacct cagccatcca |
| 241 | gaatcggttc aagtatgctc gggtcactcc tgacacagac |
| | tgggcccgct tgctgcagga |
| 301 | ccacccctgg ctgctcagcc agaacttggt agtcaagcca |
| | gaccagctga tcaaacgtcg |
| 361 | tggaaaactt ggtctcgttg gggtcaacct cactctggat |
| | ggggtcaagt cctggctgaa |
| 421 | gccacggctg ggacaggaag ccacagttgg caaggccaca |
| | ggcttcctca agaactttct |
| 481 | gatcgagccc ttcgtccccc acagtcaggc tgaggagttc |
| | tatgtctgca tctatgccac |
| 541 | ccgagaaggg gactacgtcc tgttccacca cgaggggggt |
| | gtggacgtgg gtgatgtgga |
| 601 | cgccaaggcc cagaagctgc ttgttggcgt ggatgagaaa |
| | ctgaatcctg aggacatcaa |
| 661 | aaaacacctg ttggtccacg cccctgaaga caagaaagaa |
| | attctggcca gttttatctc |
| 721 | cggcctcttc aatttctacg aggacttgta cttcacctac |
| | ctcgagatca atccccttgt |
| 781 | agtgaccaaa gatggagtct atgtccttga cttggcggcc |
| | aaggtggacg ccactgccga |
| 841 | ctacatctgc aaagtgaagt ggggtgacat cgagttccct |
| | ccccccttcg ggcgggaggc |
| 901 | atatccagag gaagcctaca ttgcagacct cgatgccaaa |
| | agtggggcaa gcctgaagct |
| 961 | gaccttgctg aaccccaaag ggaggatctg gaccatggtg |
| | gccgggggtg gcgcctctgt |
| 1021 | cgtgtacagc gataccatct gtgatctagg gggtgtcaac |
| | gagctggcaa actatgggga |
| 1081 | gtactcaggc gcccccagcg agcagcagac ctatgactat |
| | gccaagacta tcctctccct |
| 1141 | catgacccga gagaagcacc cagatggcaa gatcctcatc |
| | attggaggca gcatcgcaaa |
| 1201 | cttcaccaac gtggctgcca cgttcaaggg catcgtgaga |
| | gcaattcgag attaccaggg |
| 1261 | ccccctgaag gagcacgaag tcacaatctt tgtccgaaga |
| | ggtggcccca actatcagga |
| 1321 | gggcttacgg gtgatgggag aagtcgggaa gaccactggg |
| | atccccatcc atgtctttgg |
| 1381 | cacagagact cacatgacgg ccattgtggg catggccctg |
| | ggccaccggc ccatccccaa |
| 1441 | ccagccaccc acagcggccc acactgcaaa cttcctcctc |
| | aacgccagcg ggagcacatc |
| 1501 | gacgccagcc cccagcagga cagcatcttt ttctgagtcc |
| | agggccgatg aggtggcgcc |
| 1561 | tgcaaagaag gccaagcctg ccatgccaca agattcagtc |
| | ccaagtccaa gatccctgca |
| 1621 | aggaaagagc accaccctct tcagccgcca caccaaggcc |
| | attgtgtggg gcatgcagac |
| 1681 | ccgggccgtg caaggcatgc tggactttga ctatgtctgc |
| | tcccgagacg agccctcagt |
| 1741 | ggctgccatg gtctaccctt tcactgggga ccacaagcag |
| | aagttttact gggggcacaa |
| 1801 | agagatcctg atccctgtct tcaagaacat ggctgatgcc |
| | atgaggaagc atccggaggt |
| 1861 | agatgtgctc atcaactttg cctctctccg ctctgcctat |
| | gacagcacca tggagaccat |
| 1921 | gaactatgcc cagatccgga ccatcgccat catagctgaa |
| | ggcatccctg aggccctcac |
| 1981 | gagaaagctg atcaagaagg cggaccagaa gggagtgacc |
| | atcatcggac ctgccactgt |
| 2041 | tggaggcatc aagcctgggt gctttaagat tggcaacaca |
| | ggtgggatgc tggacaacat |
| 2101 | cctggcctcc aaactgtacc gcccaggcag cgtggcctat |
| | gtctcacgtt ccggaggcat |
| 2161 | gtccaacgag ctcaacaata tcatctctcg gaccacggat |
| | ggcgtctatg agggcgtggc |
| 2221 | cattggtggg gacaggtacc cgggctccac attcatggat |
| | catgtgttac gctatcagga |
| 2281 | cactccagga gtcaaaatga ttgtggttct tggagagatt |
| | gggggcactg aggaatataa |
| 2341 | gatttgccgg ggcatcaagg agggccgcct cactaagccc |
| | atcgtctgct ggtgcatcgg |
| 2401 | gacgtgtgcc accatgttct cctctgaggt ccagtttggc |
| | catgctggag cttgtgccaa |
| 2461 | ccaggcttct gaaactgcag tagccaagaa ccaggctttg |
| | aaggaagcag gagtgtttgt |
| 2521 | gccccggagc tttgatgagc ttggagagat catccagtct |
| | gtatacgaag atctcgtggc |
| 2581 | caatggagtc attgtacctg cccaggaggt gccgccccca |
| | accgtgccca tggactactc |
| 2641 | ctgggccagg gagcttggtt tgatccgcaa acctgcctcg |
| | ttcatgacca gcatctgcga |
| 2701 | tgagcgagga caggagctca tctacgcggg catgcccatc |
| | actgaggtct tcaaggaaga |
| 2761 | gatgggcatt ggcggggtcc tcggcctcct ctggttccag |
| | aaaaggttgc ctaagtactc |
| 2821 | ttgccagttc attgagatgt gtctgatggt gacagctgat |
| | cacgggccag ccgtctctgg |
| 2881 | agcccacaac accatcattt gtgcgcgagc tgggaaagac |
| | ctggtctcca gcctcacctc |
| 2941 | ggggctgctc accatcgggg atcggtttgg gggtgccttg |
| | gatgcagcag ccaagatgtt |
| 3001 | cagtaaagcc tttgacagtg gcattatccc catggagttt |
| | gtgaacaaga tgaagaagga |
| 3061 | agggaagctg atcatgggca ttggtcaccg agtgaagtcg |
| | ataaacaacc cagacatgcg |
| 3121 | agtgcagatc ctcaaagatt acgtcaggca gcacttccct |
| | gccactcctc tgctcgatta |
| 3181 | tgcactggaa gtagagaaga ttaccacctc gaagaagcca |
| | aatcttatcc tgaatgtaga |
| 3241 | tggtctcatc ggagtcgcat ttgtagacat gcttagaaac |
| | tgtgggtcct ttactcggga |
| 3301 | ggaagctgat gaatatattg acattggagc cctcaatggc |
| | atctttgtgc tgggaaggag |
| 3361 | tatggggttc attggacact atcttgatca gaagaggctg |
| | aagcaggggc tgtatcgtca |
| 3421 | tccgtgggat gatatttcat atgttcttcc ggaacacatg |
| | agcatgtaa |

In one embodiment, an antisense oligonucleotide that inhibits FGL2 may comprise at least 5, at least 10, at least 20, at least 30, at least 40, at least 50, at least 60, at least 70, at least 80, at least 90, at least 100, at least 200, at least 300, at least 400, at least 500, at least 600, at least 700, at least 800, at least 900, at least 1000, at least 2000, or more bases complementary to a portion of the coding sequence of the human FGL2 gene. In one embodiment, the coding sequence of the FGL2 gene is the sequence of SEQ ID NO: 38.

SEQ ID NO: 38 is the nucleotide sequence of the FGL2 gene (NM_001014342.2 Homo sapiens filaggrin family member 2 (FLG2), mRNA).

(SEQ ID NO: 38)
ACCCTGCAAGCTGCATCAGGCTTTATCCTACTTGTTCCTTTGGTGAACCAGGTTCACTTAAACTTGCAAA
AAGATGACCGACCTCTTGAGAAGTGTTGTCACCGTAATTGATGTTTTCTACAAATACACCAAGCAAGATG
GGGAGTGTGGCACACTGAGCAAGGGTGAACTAAAGGAACTTCTGGAGAAAGAGCTTCATCCAGTTCTGAA
GAACCCAGATGATCCAGACACAGTGGATGTCATCATGCATATGCTGGATCGAGATCATGACAGAAGATTG
GACTTTACTGAGTTTCTTTTGATGATATTCAAGCTGACTATGGCCTGCAACAAGGTCCTCAGCAAAGAAT
ACTGCAAAGCTTCAGGGTCAAAGAAGCATAGGCGTGGTCACCGACACCAAGAAGAAGAAAGTGAAACAGA
AGAGGATGAAGAGGATACACCAGGACATAAATCAGGTTACAGACATTCAAGTTGGAGTGAGGGAGAGGAG
CATGGATATAGTTCTGGGCACTCAAGGGGAACTGTGAAATGTAGACATGGGTCCAACTCCAGGAGGCTAG
GAAGACAAGGTAATTTATCCAGCTCTGGGAACCAAGAGGGATCTCAGAAAAGATACCACAGGTCCAGCTG
TGGTCATTCATGGAGTGGTGGCAAAGACAGACATGGTTCCAGCTCTGTAGAACTGAGAGAAAGAATAAAC
AAGTCACACATTAGCCCTTCTAGGGAATCTGGGGAGGAGTATGAATCTGGATCTGGATCAAACAGTTGGG
AAAGGAAAGGTCATGGTGGTCTGTCATGTGGATTGGAGACTAGTGGGCATGAATCAAACTCTACTCAGTC
AAGAATTAGAGAACAAAAGCTTGGGTCTAGCTGTTCAGGTTCAGGAGACAGTGGGAGGCGAAGTCATGCA
TGTGGTTATAGCAATTCAAGTGGGTGTGGAAGGCCACAAAATGCTTCAAGTTCTTGTCAGTCACATAGAT
TTGGAGGGCAAGGAAATCAATTTAGCTATATTCAGTCAGGCTGTCAGTCAGGAATTAAGGGAGGACAAGG
CCATGGCTGTGTCTCAGGAGGTCAGCCCTCTGGATGTGGTCAACCTGAGTCTAACCCCTGTAGTCAGTCC
TATAGTCAGAGAGGATATGGAGCTAGAGAAAATGGTCAACCACAGAACTGTGGAGGACAATGGAGAACAG
GCTCAAGTCAGTCCTCTTGCTGTGGACAATATGGGTCTGGAGGTAGCCAGTCTTGTAGTAATGGTCAACA
TGAATATGGTTCCTGTGGCCGCTTTTCAAACTCTTCTAGTTCAAATGAATTTTCCAAATGTGATCAATAT
GGGTCTGGTTCAAGTCAGTCTACTAGCTTTGAACAACATGGAACAGGCTTGAGTCAGTCCTCTGGGTTCG
AACAACATGTATGTGGCTCAGGTCAAACTTGTGGCCAGCATGAGTCTACATCAAGTCAATCCTTGGGCTA
TGACCAGCATGGGTCTAGCTCAGGTAAGACATCTGGCTTTGGACAACATGGGTCTGGCTCAGGTCAGTCC
TCTGGCTTTGGACAATGTGGGTCAGGCTCAGGTCAGTCCTCTGGCTTTGGACAGCATGGGTCTGTCTCAG
GACAATCCTCTGGTTTTGGACAGCATGGGTCTGTCTCAGGACAATCCTCTGGTTTTGGACAACATGAGTC
TAGATCACGTCAGTCTAGCTATGGCCAACATGGTTCTGGCTCAAGTCAATCATCTGGCTATGGCCAATAT
GGGTCTAGAGAGACATCTGGCTTTGGACAACATGGGTTGGGCTCAGGTCAATCCACTGGCTTTGGCCAAT
ATGGATCGGGCTCAGGTCAGTCCTCTGGCTTTGGACAACATGGGTCTGGCTCAGGACAATCCTCTGGCTT
TGGACAACATGAGTCTAGATCAGGTCAGTCTAGTTATGGCCAACACAGTTCTGGCTCAAGTCAGTCATCT
GGCTATGGCCAACATGGGTCTAGACAGACATCTGGCTTTGGACAACATGGGTCAGGCTCAAGTCAATCCA
CTGGCTTTGGCCAATATGGATCAGGCTCAGGTCAGTCCTCTGGCTTTGGACAACATGTTTCTGGCTCAGG
ACAATCCTCTGGTTTTGGACAACATGAGTCTAGATCAGGTCATTCTAGCTATGGCCAACATGGTTTTGGC
TCAAGTCAATCATCTGGCTATGGTCAACATGGGTCAAGTTCAGGACAGACATCTGGATTTGGACAACACG
AGTTAAGCTCAGGTCAGTCTTCCAGCTTTGGCCAACATGGATCAGGCTCAGGTCAGTCCTCTGGCTTTGG
ACAACATGGGTCTGGCTCAGGACAATCCTCTGGCTTTGGACAACATGAGTCTAGATCAGGTCAGTCTAGC
TATGGCCAACACAGTTCTGGCTCAAGTCAGTCATCTGGCTATGGCCAACATGGGTCTAGACAGACATCTG
GCTTTGGACAACATGGGTCAGGCTCAAGTCAATCCACTGGCTTTGGCCAATATGGATCAGGCTCAGGTCA
GTCCGCTGGCTTTGGACAACATGGGTCTGGCTCAGGACAATCCTCTGGCTTTGGACAGCATGAGTCTAGA
TCACATCAGTCCAGCTATGGCCAACATGGTTCTGGCTCAAGTCAATCATCTGGCTATGGTCAACATGGGT
CAAGTTCGGGACAGACATCTGGCTTTGGACAACACAGGTCAAGCTCAGGTCAATACTCTGGCTTTGGACA
ACATGGATCAGGCTCAGGTCAGTCCAGTGGCTTTGGACAACATGGGACTGGCTCAGGACAATACTCTGGT
TTTGGACAACATGAGTCTAGATCACATCAGTCTAGCTATGGCCAACATGGTTCTGGCTCAAGTCAGTCAT
CTGGCTATGGTCAACATGGGTCAAGTTCAGGACAGACTTTTGGATTTGGACAACACAGGTCAGGCTCAGG
TCAATCCTCTGGCTTTGGCCAACATGGATCAGGCTCAGGTCAGTCCTCTGGCTTTGGACAACATGAGTCA
GGCTCAGGAAAATCCTCTGGCTTTGGACAGCATGAGTCTAGATCAAGTCAGTCTAATTATGGCCAACATG
GTTCTGGCTCAAGTCAGTCATCTGGCTATGGTCAACATGGGTCTAGTTCAGGACAGACAACTGGCTTTGG
ACAACACAGGTCAAGCTCAGGCCAATACTCAGGCTTTGGACAACATGGATCAGGCTCAGATCAGTCCTCT
GGCTTTGGACAACATGGGACTGGTTCAGGACAATCCTCTGGTTTTGGACAATATGAGTCTAGATCACGTC
AGTCTAGCTATGGCCAACATGGTTCTGGCTCAAGTCAATCATCTGGCTATGGTCAACATGGGTCAAATTC
AGGACAGACATCTGGATTTGGACAACACAGGCCAGGCTCAGGTCAGTCCTCTGGCTTTGGCCAATATGGA
TCGGGCTCAGGTCAGTCTTCTGGCTTTGGACAACATGGGTCAGGCACAGGTAAATCCTCTGGCTTTGCAC
AGCATGAGTACAGATCAGGTCAGTCTAGCTATGGCCAACATGGTACTGGCTCCAGTCAATCATCTGGCTG
TGGCCAACATGAGTCTGGCTCAGGTCCAACCACAAGTTTTGGACAGCATGTGTCTGGCTCAGACAATTTC
TCTAGTTCTGGACAACATATATCTGACTCAGGTCAGTCCACTGGATTTGGCCAATATGGTTCAGGCTCAG
GTCAATCAACTGGCTTGGGCCAGGGTGAATCTCAACAAGTAGAGTCAGGATCCACAGTTCATGGGAGACA
GGAAACTACTCATGGTCAGACAATAAATACCACTAGACATAGCCAGTCTGGTCAAGGACAATCCACACAG
ACAGGGTCCAGGGTAACTAGAAGACGAAGATCTAGCCAAAGTGAGAACAGTGACAGTGAAGTGCACTCAA
AGGTCTCACACAGACATTCAGAACACATTCACACACAAGCTGGATCTCACTACCCAAAGTCAGGATCCAC
AGTTCGCAGAAGACAAGGAACTACTCATGGACAGAGAGGAGATACCACTAGACATGGCCATTCTGGTCAT
GGACAGTCTACACAGACAGGTTCCAGAACATCTGGAAGACAGAGATTTAGCCACAGTGATGCCACTGACA
GTGAAGTGCACTCAGGGGTCTCACATAGACCACACTCACAAGAACAAACTCACAGCCAAGCTGGATCTCA
ACATGGAGAGTCAGAATCCACAGTTCATGAGAGACATGAAACTACTTATGGACAGACAGGAGAGGCCACT
GGACATGGCCACTCTGGTCATGGACAGTCCACACAGAGAGGGTCCAGGACAACTGGAAGAAGGGGATCTG
GCCATAGTGAGTCCAGTGACAGTGAAGTGCACTCAGGGGGCTCACACAGACCACAATCACAAGAACAAAC
TCATGGCCAAGCCGGATCTCAACATGGAGAGTCAGGATCCACAGTTCATGGGAGACACGGAACTACTCAT
GGACAGACAGGAGATACCACTAGACATGCCCACTATCATCATGGAAAATCCACACAGAGAGGGTCCAGTA
CAACTGGAAGAAGGGGATCTGGCCACAGTGAGTCCAGTGACAGTGAAGTGCACTCAGGGGGCTCGCACAC
ACATTCAGGACACACTCACGGCCAAAGTGGATCTCAACATGGAGAGTCAGAATCCATAATTCATGACAGA
CACAGAATTACTCATGGACAGACAGGAGATACCACTAGACATTCCTACTCTGGTCATGAACAAACCACAC
AGACAGGGTCCAGGACAACTGGAAGACAGAGAACTAGCCACAGTGAGTCCACTGACAGTGAAGTGCACTC
AGGGGGCTCACACAGACCACACTCACGAGAACACACTTACGGCCAAGCCGGATCTCAACATGAAGAGCCA
GAATTCACAGTTCATGAGAGACACGGAACTACTCATGGACAGATAGGAGATACCACTGGACATTCCCACT
CTGGTCATGGACAGTCCACACAGAGAGGGTCCAGGACAACTGGAAGACAGAGATCTAGCCACAGTGAGTC
CAGTGACAGTGAAGTGCACTCAGGGGTCTCACACACACATACAGGACACACTCATGGTCAAGCTGGATCT
CAACATGGACAGTCAGAATCCATAGTTCCTGAGAGACATGGAACTACTCATGGACAGACAGGAGATACCA
CTAGACATGCCCACTATCATCATGGATTAACCACACAGACAGGGTCCAGGACTACTGGAAGAAGGGGATC
TGGCCACAGTGAGTACAGTGACAGTGAAGGGTACTCAGGAGTCTCACATACACATTCAGGACACACTCAT
GGCCAAGCCAGATCTCAACATGGAGAGTCAGAATCCATAGTTCATGAGAGACATGGAACTATACATGGAC
AGACAGGCGATACCACCAGACATGCCCACTCTGGTCATGGACAGTCCACACAGACAGGGTCCAGGACCAC
TGGAAGAAGGTCATCTGGCCACAGTGAGTACAGTGACAGTGAAGGGCACTCAGGGTTCTCACAAAGACCA
CACTCACGAGGACACACTCACGGCCAGGCTGGATCTCAACATGGAGAGTCAGAATCCATAGTTGACGAGA
GACATGGAACTACTCATGGACAGACAGGAGATACCAGTGGACATTCTCAATCTGGTCATGGACAGTCCAC
ACAGTCAGGATCCAGTACAACTGGAAGAAGGAGATCTGGCCACAGTGAGTCCAGTGACAGTGAAGTGCAC
TCAGGGGGCTCACATACACATTCAGGACACACACACAGCCAAGCCAGGTCTCAACATGGAGAGTCAGAAT
CCACAGTTCACAAGAGACACCAAACTACTCATGGACAGACAGGAGATACCACTGAACATGGCCACCCTAG
TCATGGACAAACCATACAGACAGGGTCCAGGACAACTGGAAGAAGGGGATCTGGCCACAGTGAGTACAGT
GACAGTGAAGGGCCCTCAGGGGTCTCACACACACATTCAGGACACACTCACGGTCAAGCTGGATCTCACT
ATCCAGAGTCAGGATCCTCAGTTCATGAGAGACACGGAACTACTCATGGACAAACAGCAGATACCACTAG
ACATGGCCACTCTGGTCATGGACAGTCCACACAGAGAGGGTCCAGGACAACTGGAAGAAGGGCATCTGGC
CACAGTGAGTACAGTGACAGTGAAGGGCACTCAGGGGTCTCACACACACATTCAGGACACGCTCATGGCC
AAGCCGGATCTCAACATGGAGAGTCAGGATCCTCAGTTCATGAGAGACACGGAACTACTCATGGACAGAC
AGGAGATACCACTAGACATGCTCACTCTGGTCATGGACAGTCCACACAGAGAGGGTCAAGGACAGCTGGA
AGAAGGGGATCTGGCCACAGTGAGTCCAGTGACAGTGAAGTGCACTCAGGGGTCTCACACACACATTCAG
GACACACTTATGGCCAAGCCAGATCTCAACATGGAGAGTCAGGATCTGCCATTCACGGGAGACAGGGAAC
TATACATGGACAGACAGGAGATACCACTAGACATGGCCAGTCTGGTCATGGACAGTCCACACAGACAGGT
TCCAGGACAACTGGAAGACAAAGATCTAGTCACAGTGAGTCCAGTGATAGTGAAGTGCACTCAGAGGCCT
CACCCACACATTCAGGACACACTCACAGCCAAGCCGGATCTCGACATGGACAGTCAGGATCCTCAGGTCA
TGGGAGACAGGGAACTACTCATGGACAGACAGGAGATACCACTAGACATGCCCACTATGGTTATGGACAA
TCCACACAGAGAGGGTCCAGGACAACTGGAAGAAGGGGATCTGGCCACAGTGAGTCCAGTGACAGTGAAG
TGCACTCATGGGGCTCACACACACATTCAGGACACATTCAGGGCCAAGCTGGATCTCAACAAAGACAGCC
AGGATCCACAGTTCATGGGAGACTGGAAACTACTCATGGACAGACAGGAGATACCACTAGACATGGCCAT
TCTGGTTATGGACAATCCACACAGACAGGTTCCAGATCTAGTAGAGCAAGTCATTTTCAGTCACATAGTA
GTGAAAGGCAAAGGCATGGATCAAGTCAGGTTTGGAAACATGGCAGCTATGGACCTGCAGAATATGACTA
TGGGCACACTGGGTATGGGCCTTCTGGTGGCAGCAGAAAAAGCATCAGTAATTCTCACCTTTCATGGTCA
ACAGACAGCACTGCAAACAAGCAACTGTCTAGACATTGACAGTTATTTTCTAGTTCTGACCTTATAGTAT
CCAAAGCAACTAAAAGAACAGGAAGACACAGTTTAAATCATGAACAGTCAATGGTAAGTTATGAACATTC
AGTTGATTCTCAGTATCAGTCTCGACCTATTATTATAAGAAGTCAGGAATCTAGTCATGGACATTCTATA
GTAACTCATAAACAGTCAAACAACACCTATGTTCAACCTGGATATAACACAGCCAGAAGGGAGGGATGTA
CACATAGCCAGTCAAATGACCACCTTGGATTTGGCCATGGACAATCCATATCAGTTCATGGCCATTCAAA
ATCTAGTTCAATCAGAAAACAGGAATCCCATACTGATAACAAAAAGCATTCAGAAGATTGGGAGAAAGAC
ACTCATGAGCAATTAGGATCTAGGCATGGGAAGTTAGAGTTCAATACAATAGGTATACATGGATCTAGCC
AGCAACATTTCGGAGATACAACTTTTCATGGGCAGGTAAGATCCAGCACAGGTTTTGCCAGATAGGTATT
AAGTCATGGGCCATCAAGAGATGCCTAGGGTCAGTCTGGATTCAGTACCAATGAAAGACAAGTATACAGC
CATGGCCAATCAAATGATAGTTATGAGTAGTCAAATGACAGCAAAAGTCAAAGATACATTTTCAGTCACT
TTCTTGACAGCCAAGACCCTGCAGGAATTGAAGAGTATAGGTATAGATATTCATCAAGCAGTGCAACCAT
ATGCAGTGGGGGAGACAAAGGCAAGAGTCAGAGTCAAGTCTGTCAGGAGGTATCAGAATATACGGTGAGG
ATGTGGGCAAAAAACAAAGAGGCTCTGAGGCCAGCGGTTACCATACAAAGGAAAGAACAGGCTCTGGTTC
CTTCTGCTTAGATAGCAACACCCCACTCTATGAATATGTCCAAGAACAAAGGAGTTATTACTTTGAATAA
GAAGCCAACATAAACTAGCCCAAGATAAGAACTAACCCAGAGAAGAAATGAGACACATACATGAAATTAA
GGTATTTAACATGATCTCCTCTTTTGGTAGTAAGGGTATATGTCTGTTCTTTCATTTTAACTATAGTTCT
GTACTATATTACTTTTGTTTGGTGCCAGGTGTTTTTGTAAGGCTCCACATTCATTGAACTCCTTGGTTAG
AAAATAGTGAATGAGGCCGGGCGCGGTGGCTCACGCCTGTAATCCCAGCACTTTGGGAGGCCAAGATGGG
TGGATCACGAAGTCAGGAGTTCGAGACCAGCCTGGCCAACATAGTGAAATCCCGTCTCTACTAAAAATAC
AAAAATTAGCTGGGTGCAGTAGCGGGCACCTGTAATCCTAGCTACTCAGGAGGCTGAGGCAGGAGAATTG
GTTGAACCCGGGAGGCAGAGGTTGCAGTGAGCCGAGATCGCACCACTGCACTCCAGCCTGGGTGACTGAG
CAAGACTCCACCTTGAAAAAAAGAAAGAAAGAAAATAGTGAATGGAAGAAAAAGATAAACACCATTTGGG
GCTATATTCAGAACTATATAATAAAGAGAGATTTGTGTGGTTGGGAATAAAATTAGGTTTTAAAAAAATT
TCCGAATTTCAAAATTTGGATTTTCCAGATTTTGTTTTAATTAATTATTGACAAACCTATCCAAGGAGCT
AAATGACGTGGTTTAGGAGCCAAAACGTCTCTAGAAACATTATAACACATCCCATCCTGAACAAAGAATG
TTGTCATAGTTTCCCACATGAATATTCAGAATATTTGCATCTTTTTGTGTCACTACTGGAATTCTGTACA
ATTATATTTAAATTTATTGTCATGGCCTTCTGGATATAGGATCCAGAAAATTGTACTTCATAAAAATTGG
CAATAAACATTTCATCAAGTTAAAAAAAAAAAAAAAAAAAAAAAAAAAAAAAAAAAAAAAAAAAAAAAAA

In one embodiment, an antisense oligonucleotide that inhibits SFPQ may comprise at least 5, at least 10, at least 20, at least 30, at least 40, at least 50, at least 60, at least 70, at least 80, at least 90, at least 100, at least 200, at least 300, at least 400, at least 500, at least 600, at least 700, at least 800, at least 900, at least 1000, at least 2000, or more bases complementary to a portion of the coding sequence of the human SFPQ gene. In one embodiment, the coding sequence of the SFPQ gene is the sequence of SEQ ID NO: 39.

SEQ ID NO: 39 is the nucleotide sequence of the SFPQ gene (mRNA (cDNA clone MGC:57191 IMAGE:5262885), complete cds).

(SEQ ID NO: 39)
TCTGTGTCATCCGCCATTTTGTGAGAAGCAAGGTGGCCTCCACGTTTCCT
GAGCGTCTTCTTCGCTTTTGCCTCGACCGCCCCTTGACCACAGACATGTC
TCGGGATCGGTTCCGGAGTCGTGGCGGTGGCGGTGGTGGCTTCCACAGGC
GTGGAGGAGGCGGCGGCCGCGGCGGCCTCCACGACTTCCGTTCTCCGCCG
CCCGGCATGGGCCTCAATCAGAATCGCGGCCCCATGGGTCCTGGCCCGGG
CCAGAGCGGCCCTAAGCCTCCGATCCCGCCACCGCCTCCACACCAACAGC
AGCAACAGCCACCACCGCAGCAGCCACCGCCGCAGCAGCCGCCACCGCAT
CAGCCGCCGCCGCATCCACAGCCGCATCAGCAGCAGCAGCCGCCGCCACC
GCCGCAGGACTCTTCCAAGCCCGTCGTTGCTCAGGGACCCGGCCCCGCTC
CCGGAGTAGGCAGCACACCACCAGCCTCCAGCTCGGCCCCGCCCGCCACT
CCACCAACCTCGGGGGCCCCGCCAGGGTCCGGGCCAGGCCCGACTCCGAC
CCCGCCGCCTGCAGTCACCTCGGCCCCTCCCGGGGCGCCGCCACCCACCC
CGCCAAGCAGCGGGGTCCCTACCACACCTCCTCAGGCCGGAGGCCCGCCG
CCTCCGCCCGCGGCAGTCCCGGGCCCGGGTCCAGGGCCTAAGCAGGGCCC
AGGTCCGGGTGGTCCCAAAGGCGGCAAAATGCCTGGCGGGCCGAAGCCAG
GTGGCGGCCCGGGCCTAAGTACGCCTGGCGGCCACCCCAAGCCGCCGCGT
CGAGGCGGCGGGGAGCCCCGCGGGGGCCGCCAGCACCACCCGCCCTACCA
CCAGCAGCATCACCAGGGGCCCCCGCCCGGCGGGCCCGGCGGCCGCAGCG
AGGAGAAGATCTCGGACTCGGAGGGGTTTAAAGCCAATTTGTCTCTCTTG
AGGAGGCCTGGAGAGAAAACTTACACACAGCGATGTCGGTTGTTTGTTGG
GAATCTACCTGCTGATATCACGGAGGATGAATTCAAAAGACTATTTGCTA
AATATGGAGAACCAGGAGAAGTTTTTATCAACAAAGGCAAAGGATTCGGA
TTTATTAAGCTTGAATCTAGAGCTTTGGCTGAAATTGCCAAAGCCGAACT
GGATGATACACCCATGAGAGGTAGACAGCTTCGAGTTCGCTTTGCCACAC
ATGCTGCTGCCCTTTCTGTTCGTAATCTTTCACCTTATGTTTCCAATGAA
CTGTTGGAAGAAGCCTTTAGCCAATTTGGTCCTATTGAAAGGGCTGTTGT
AATAGTGGATGATCGTGGAAGATCTACAGGGAAAGGCATTGTTGAATTTG
CTTCTAAGCCAGCAGCAAGAAAGGCATTTGAACGATGCAGTGAAGGTGTT
TTCTTACTGACGACAACTCCTCGTCCAGTCATTGTGGAACCACTTGAACA
ACTAGATGATGAAGATGGTCTTCCTGAAAAACTTGCCCAGAAGAATCCAA
TGTATCAAAAGGAGAGAGAAACCCCTACTCGTTTTGCCCAGCATGGCACG
TTTGAGTACGAATATTCTCAGCGATGGAAGTCTTTGGATGAAATGGAAAA
ACAGCAAAGGGAACAAGTTGAAAAAAACATGAAAGATGCAAAAGACAAAT
TGGAAAGTGAAATGGAAGATGCCTATCATGAACATCAGGCAAATCTTTTG
CGCCAAGATCTGATGAGACGACAGGAAGAATTAAGACGCATGGAAGAACT
TCACAATCAAGAAATGCAGAAACGTAAAGAAATGCAATTGAGGCAAGAGG
AGGAACGACGTAGAAGAGAGGAAGAGATGATGATTCGTCAACGTGAGATG
GAAGACCAAATGAGGCGCCAAAGAGAGGAAAGTTACAGCCGAATGGGCTA
CATGGATCCACGGGAAAGAGACATGCGAATGGGTGGCGGAGGAGCAATGA
ACATGGGAGATCCCTATGGTTCAGGAGGCCAGAAATTTCCACCTCTAGGA
GGTGGTGGTGGCATAGGTTATGAAGCTAATCCTGGCGTTCCACCAGCAAC
CATGAGTGGTTCCATGATGGGAAGTGACATGCGTACTGAGCGCTTTGGGC
AGGGAGGTGCGGGGCCTGTGGGTGGACAGGGTCCTAGAGGAATGGGGCCT
GGAACTCCAGCAGGATATGGTAGAGGGAGAGAAGAGTACGAAGGCCCAAA
CAAAAAACCCCGATTTTAGATGTGATATTTAGGCTTTCATTCCAGTTTGT
TTTGTTTTTTTGTTTAGATACCAATCTTTTAAATTCTTGCATTTTAGTAA
GAAAGCTATCTTTTTATGGATGTTAGCAGTTTATTGACCTAATATTTGTA
AATGGTCTGTTTGGGCAGGTAAAATTATGTAATGCAGTGTTTGGAACAGG
AGAATTTTTTTTTCCTTTTTATTTCTTTATTTTTTCTTTTTTACTGTATA
ATGTCCCTCAAGTTTATGGCAGTGTACCTTGTGCCACTGAATTTCCAAAG
TGTACCAATTTTTTTTTTTTTACTGTGCTTCAAATAAATAGAAAATAGTT
ATAAAAAAAAAAAAAAAAAAAAAAAAAAAAAAAAAAAAAAAAAAAAAAAA
AAAAAAAAAAAAAAAAAAAAAA

In one embodiment, the desired target (e.g., a lnc-RNA expressed in a macrophage, or a protein bound to a lnc-RNA, for example, ACLY) is depleted from the cell's genome using any genome editing system including, but not limited to, zinc finger nucleases, TALENS, meganucleases, and CRISPR/Cas systems. In one embodiment, the genomic editing system used to incorporate the nucleic acid encoding one or more guide RNAs into the cell's genome is not a CRISPR/Cas system; this can prevent undesirable cell death in cells that retain a small amount of Cas enzyme/protein. It is also contemplated herein that either the Cas enzyme or the sgRNAs are each expressed under the control of a different inducible promoter, thereby allowing temporal expression of each to prevent such interference.

When a nucleic acid encoding one or more sgRNAs and a nucleic acid encoding an RNA-guided endonuclease each need to be administered in vivo, the use of an adenovirus associated vector (AAV) is specifically contemplated. Other vectors for simultaneously delivering nucleic acids to both components of the genome editing/fragmentation system (e.g., sgRNAs, RNA-guided endonuclease) include lentiviral vectors, such as Epstein Barr, Human immunodeficiency virus (HIV), and hepatitis B virus (HBV). Each of the components of the RNA-guided genome editing system (e.g., sgRNA and endonuclease) can be delivered in a separate vector as known in the art or as described herein.

In one embodiment, the desired target (e.g., a lnc-RNA expressed in a macrophage, or a protein bound to a lnc-RNA, for example, ACLY) is inhibited by RNA inhibition. In one embodiment, the nucleic acid molecule is an antisense RNA, an RNA interference (RNAi) molecule, an RNA decoy molecule, or an RNAaptamer. Inhibitors of the expression of a given gene can be an inhibitory nucleic acid. In some embodiments of any of the aspects, the inhibitory nucleic acid is an inhibitory RNA (iRNA). The RNAi can be single stranded or double stranded.

The iRNA can be siRNA, shRNA, endogenous microRNA (miRNA), or artificial miRNA. In one embodiment, an iRNA as described herein effects inhibition of the expression and/or activity of a target, e.g. a lnc-RNA expressed in a macrophage, or a protein bound to a lnc-RNA, for example, ACLY.

One skilled in the art would be able to design siRNA, shRNA, or miRNA to the desired target, e.g., a lnc-RNA expressed in a macrophage, or ACLY, e.g., using publically available design tools. siRNA, shRNA, or miRNA is commonly made using companies such as Dharmacon (Layfayette, Colo.) or Sigma Aldrich (St. Louis, Mo.).

In one embodiment, the siRNA oligo that inhibits lnc-FAM164A1 has a sequence of SEQ ID NO: 5 or 7.

In one embodiment, the siRNA oligo that inhibits FLG2 has a sequence of SEQ ID NO: 40-45. Target site: 267-292, Sense: 5′ AUGACAGAAGAUUGGACUUUACUGA 3′ (SEQ ID NO: 40); Target site: 267-292, Antisense: 5′ UCAGUAAAGUCCAAUCUUCUGUCAUGA 3′ (SEQ ID NO: 41); Target site 683-708, Sense 5′ CUGAGAGAAAGAAUAAACAAGUCAC 3′ (SEQ ID NO: 42); Target site 683-708, Antisense 5′ GUGACUUGUUUAUUCUUUCUCUCAGUU 3′ (SEQ ID NO: 43); Target site: 837-862, Sense: 5′ AGUCAAGAAUUAGAGAACAAAAGCT 3′ (SEQ ID NO: 44); Target site: 837-862, Antisense: 5′ AGCUUUUGUUCUCUAAUUCUUGACUGA 3′ (SEQ ID NO: 45).

In one embodiment, the siRNA oligo that inhibits SFPQ has a sequence of SEQ ID NO: 46-51. Target site: 1387-1412, Sense 5′ GCAGUGAAGGUGUUUUCUUACUGAC 3′ (SEQ ID NO: 46); Target site: 1387-1412, Antisense 5′ GUCAGUAAGAAAACACCUUCACUGCAU 3′ (SEQ ID NO: 47); Target sites: 2289-2314, Sense: 5′ GCAUUUUAGUAAGAAAGCUAUCUTT 3′ (SEQ ID NO: 48); Target sites: 2289-2314, Antisense: 5′ AAAGAUAGCUUUCUUACUAAAAUGCAA 3′ (SEQ ID NO: 49); Target site: 1736-1761, Sense: 5′ ACGCAUGGAAGAACUUCACAAUCAA 3′ (SEQ ID NO: 50); Target site: 1736-1761, Antisense: 5′ UUGAUUGUGAAGUUCUUCCAUGCGUCU 3′ (SEQ ID NO: 51).

In some embodiments of any of the aspects, the iRNA can be a dsRNA. A dsRNA includes two RNA strands that are sufficiently complementary to hybridize to form a duplex structure under conditions in which the dsRNA will be used. One strand of a dsRNA (the antisense strand) includes a region of complementarity that is substantially complementary, and generally fully complementary, to a target sequence. The target sequence can be derived from the sequence of an mRNA formed during the expression of the target. The other strand (the sense strand) includes a region that is complementary to the antisense strand, such that the two strands hybridize and form a duplex structure when combined under suitable conditions.

The RNA of an iRNA can be chemically modified to enhance stability or other beneficial characteristics. The nucleic acids featured in the invention may be synthesized and/or modified by methods well established in the art, such as those described in “Current protocols in nucleic acid chemistry,” B eaucage, S. L. et al. (Edrs.), John Wiley & Sons, Inc., New York, N.Y., USA, which is hereby incorporated herein by reference.

In one embodiment, the agent is miRNA that inhibits the desired target, e.g., a lnc-RNA expressed in a macrophage, or a protein bound to a lnc-RNA, for example, ACLY. MicroRNAs are small non-coding RNAs with an average length of 22 nucleotides. These molecules act by binding to complementary sequences within mRNA molecules, usually in the 3′ untranslated (3′UTR) region, thereby promoting target mRNA degradation or inhibited mRNA translation. The interaction between microRNA and mRNAs is mediated by what is known as the “seed sequence”, a 6-8-nucleotide region of the microRNA that directs sequence-specific binding to the mRNA through imperfect Watson-Crick base pairing. More than 900 microRNAs are known to be expressed in mammals. Many of these can be grouped into families on the basis of their seed sequence, thereby identifying a “cluster” of similar microRNAs. A miRNA can be expressed in a cell, e.g., as naked DNA. A miRNA can be encoded by a nucleic acid that is expressed in the cell, e.g., as naked DNA or can be encoded by a nucleic acid that is contained within a vector.

In one embodiment, the agent, e.g., the miRNA, has a sequence corresponding to the sequence of SEQ ID NO: 1; or comprises the sequence of SEQ ID NO: 1; or comprises a sequence with at least 80%, at least 85%, at least 90%, at least 91%, at least 92%, at least 93%, at least 94%, at least 95%, at least 96%, at least 97%, at least 98%, at least 99%, or at least 100% sequence identity to the sequence of SEQ ID NO: 1, and having the same activity as the sequence of SEQ ID NO: 1 (e.g., inhibits lnc-FAM164A1).

In one embodiment, the agent, e.g., the miRNA, has a sequence corresponding to the sequence of SEQ ID NO: 2, or comprises the sequence of SEQ ID NO: 2; or comprises a sequence with at least 80%, at least 85%, at least 90%, at least 91%, at least 92%, at least 93%, at least 94%, at least 95%, at least 96%, at least 97%, at least 98%, at least 99%, or at least 100% sequence identity to the sequence of SEQ ID NO: 2, and having the same activity as the sequence of SEQ ID NO: 2 (e.g., inhibits ACLY).

The agent may result in gene silencing of the target gene (e.g., a lnc-RNA expressed in a macrophage, or a protein bound to a lnc-RNA, for example, ACLY), such as with an RNAi molecule (e.g. siRNA or miRNA). This entails a decrease in the mRNA level in a cell for a target by at least about 5%, about 10%, about 20%, about 30%, about 40%, about 50%, about 60%, about 70%, about 80%, about 90%, about 95%, about 99%, about 100% of the mRNA level found in the cell without the presence of the agent. In one preferred embodiment, the mRNA levels are decreased by at least about 70%, about 80%, about 90%, about 95%, about 99%, about 100%. One skilled in the art will be able to readily assess whether the siRNA, shRNA, or miRNA effective target e.g., a lnc-RNA expressed in a macrophage, or ACLY, for its downregulation, for example by transfecting the siRNA, shRNA, or miRNA into cells and detecting the levels of a gene (e.g., a lnc-RNA expressed in a macrophage, or ACLY) found within the cell via PCR-based assays as compared to an appropriate control. As used herein, the appropriate control is a cell that is not treated with a iRNA or RNAi molelcule, or treated with a non-targeting iRNA or RNAi molelcule (e.g., a control iRNA or RNAi molelcule).

The agent may be contained in and thus further include a vector. Many such vectors useful for transferring exogenous genes into target mammalian cells are available. The vectors may be episomal, e.g. plasmids, virus-derived vectors such cytomegalovirus, adenovirus, etc., or may be integrated into the target cell genome, through homologous recombination or random integration, e.g. retrovirus-derived vectors such as MMLV, HIV-1, ALV, etc. In some embodiments, combinations of retroviruses and an appropriate packaging cell line may also find use, where the capsid proteins will be functional for infecting the target cells. Usually, the cells and virus will be incubated for at least about 24 hours in the culture medium. The cells are then allowed to grow in the culture medium for short intervals in some applications, e.g. 24-73 hours, or for at least two weeks, and may be allowed to grow for five weeks or more, before analysis. Commonly used retroviral vectors are “defective”, i.e. unable to produce viral proteins required for productive infection. Replication of the vector requires growth in the packaging cell line.

The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, artificial chromosome, virus, virion, etc.

As used herein, the term “expression vector” refers to a vector that directs expression of an RNA or polypeptide described herein from nucleic acid sequences contained therein linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

Integrating vectors have their delivered RNA/DNA permanently incorporated into the host cell chromosomes. Non-integrating vectors remain episomal which means the nucleic acid contained therein is never integrated into the host cell chromosomes. Examples of integrating vectors include retroviral vectors, lentiviral vectors, hybrid adenoviral vectors, and herpes simplex viral vector.

One example of a non-integrative vector is a non-integrative viral vector. Non-integrative viral vectors eliminate the risks posed by integrative retroviruses, as they do not incorporate their genome into the host DNA. One example is the Epstein Barr oriP/Nuclear Antigen-1 (“EBNA1”) vector, which is capable of limited self-replication and known to function in mammalian cells. As containing two elements from Epstein-Barr virus, oriP and EBNA1, binding of the EBNA1 protein to the virus replicon region oriP maintains a relatively long-term episomal presence of plasmids in mammalian cells. This particular feature of the oriP/EBNA1 vector makes it ideal for generation of integration-free cells. Another non-integrative viral vector is adenoviral vector and the adeno-associated viral (AAV) vector.

Another non-integrative viral vector is RNA Sendai viral vector, which can produce protein without entering the nucleus of an infected cell. The F-deficient Sendai virus vector remains in the cytoplasm of infected cells for a few passages, but is diluted out quickly and completely lost after several passages (e.g., 10 passages).

Another example of a non-integrative vector is a minicircle vector. Minicircle vectors are circularized vectors in which the plasmid backbone has been released leaving only the eukaryotic promoter and cDNA(s) that are to be expressed.

As used herein, the term “viral vector” refers to a nucleic acid vector construct that includes at least one element of viral origin and has the capacity to be packaged into a viral vector particle. The viral vector can contain a nucleic acid encoding a polypeptide as described herein in place of non-essential viral genes. The vector and/or particle may be utilized for the purpose of transferring nucleic acids into cells either in vitro or in vivo. Numerous forms of viral vectors are known in the art.

In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of ordinary skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity, e.g. ligand-mediated receptor activity and specificity of a native or reference polypeptide is retained.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In some embodiments, a polypeptide described herein (or a nucleic acid encoding such a polypeptide) can be a functional fragment of one of the amino acid sequences described herein. As used herein, a “functional fragment” is a fragment or segment of a peptide which retains at least 50% of the wildtype reference polypeptide's activity according to an assay known in the art or described below herein. A functional fragment can comprise conservative substitutions of the sequences disclosed herein.

In some embodiments, a polypeptide described herein can be a variant of a polypeptide or molecule as described herein. In some embodiments, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity of the non-variant polypeptide. A wide variety of PCR-based site-specific mutagenesis approaches are known in the art and can be applied by the ordinarily skilled artisan.

A variant amino acid or DNA sequence can be at least 80%, at least 90%, at least 91%, at least 92%, at least 93%, at least 94%, at least 95%, at least 96%, at least 97%, at least 98%, at least 99%, identical to a native or reference sequence. The degree of homology (percent identity) between a native and a mutant sequence can be determined, for example, by comparing the two sequences using freely available computer programs commonly employed for this purpose on the world wide web (e.g. BLASTp or BLASTn with default settings).

Alterations of the native amino acid sequence can be accomplished by any of a number of techniques known in the art. Mutations can be introduced, for example, at particular loci by synthesizing oligonucleotides containing a mutant sequence, flanked by restriction sites permitting ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are well established and include, for example, those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties. Any cysteine residue not involved in maintaining the proper conformation of a polypeptide also can be substituted, generally with serine, to improve the oxidative stability of the molecule and prevent aberrant crosslinking. Conversely, cysteine bond(s) can be added to a polypeptide to improve its stability or facilitate oligomerization.

### Pharmaceutical Compositions

One aspect provides a composition comprising any of the agents described herein and a pharmaceutically acceptable carrier. In one embodiment, the composition comprises at least one agent described herein.

In one embodiment, the composition is formulated for the treatment or prevention of an inflammatory disease.

In another embodiment, the pharmaceutical composition is administered to a subject with an inflammatory disease in need thereof, whereby the pharmaceutical composition decreases at least one symptom of the disease by 50%.

In one embodiment, the pharmaceutical composition is administered to a subject at risk of having an inflammatory disease.

The pharmaceutical composition can further comprise a lipid vehicle. Exemplary lipid vehicles include, but are not limited to, liposomes, micelles, exosomes, lipid emulsions, and lipid-drug complex.

In one embodiment, the composition further comprises a particle or polymer-based vehicle. Exemplary particle or polymer-based vehicles include, but are not limited to, nanoparticles, microparticles, polymer microspheres, or polymer-drug conjugates.

### Administration

In some embodiments, the methods described herein relate to treating a subject having or diagnosed as having an inflammatory disease comprising administering an agent that inhibits a target, e.g., lnc-RNA expressed in a macrophage, lnc-FAM164A1, or a protein bound to a lnc-RNA, for example, ACLY, as described herein. Subjects having an inflammatory disease can be identified by a physician using current methods of diagnosing a condition. Symptoms and/or complications an inflammatory disease, which characterize this disease and aid in diagnosis are well known in the art and include but are not limited to, fever, fatigue, pain, rashes, vomiting, shortness of breath, gastrointestinal discomfort, cardiovascular complications, or an increase or drop in blood pressure. Tests that can aid in a diagnosis of, e.g. an inflammatory disease, include but are not limited example blood tests, non-invasive imaging, and/or tissue biopsy. A family history of an inflammatory disease will also aid in determining if a subject is likely to have the condition or in making a diagnosis of an inflammatory disease.

The agents and compositions described herein (e.g., that inhibit a target described herein) can be administered to a subject having or diagnosed as having an inflammatory disease. In some embodiments, the methods described herein comprise administering an effective amount of an agent to a subject in order to alleviate at least one symptom of the inflammatory disease. As used herein, “alleviating at least one symptom of the inflammatory disease” is ameliorating any condition or symptom associated with the disease (e.g., fever, fatigue, pain, rashes, vomiting, shortness of breath, etc.). As compared with an equivalent untreated control, such reduction is by at least 5%, 10%, 20%, 40%, 50%, 60%, 80%, 90%, 95%, 99% or more as measured by any standard technique. A variety of means for administering the agents and compositions described herein to subjects are known to those of skill in the art.

In one embodiment, the agent is administered systemically or locally (e.g., to the target organ). In one embodiment, the agent is administered intravenously. In one embodiment, the agent is administered continuously, in intervals, or sporadically. The route of administration of the agent will be optimized for the type of agent being delivered (e.g., a miRNA, a small molecule, or an RNAi), and can be determined by a skilled practitioner.

The term “effective amount” as used herein refers to the amount of an agent or composition thereof described herein can be administered to a subject having or diagnosed as having an inflammatory disease needed to alleviate at least one or more symptom of the disease. The term “therapeutically effective amount” therefore refers to an amount of an agent or composition that is sufficient to provide a particular anti-inflammatory disease effect when administered to a typical subject. An effective amount as used herein, in various contexts, would also include an amount of an agent sufficient to delay the development of a symptom of the disease, alter the course of a symptom of the disease (e.g., slowing the progression of the inflammatory disease), or reverse a symptom of the disease (e.g., correcting or halting symptoms of the inflammatory disease). Thus, it is not generally practicable to specify an exact “effective amount”. However, for any given case, an appropriate “effective amount” can be determined by one of ordinary skill in the art using only routine experimentation.

In one embodiment, the agent or composition is administered continuously (e.g., at constant levels over a period of time). Continuous administration of an agent can be achieved, e.g., by epidermal patches, continuous release formulations, or on-body injectors.

In one embodiment, the agent or composition is administered in intervals (e.g., at various levels over a given period of time).

Effective amounts, toxicity, and therapeutic efficacy can be evaluated by standard pharmaceutical procedures in cell cultures or experimental animals. The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of the agent, which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay, e.g., measuring neurological function, or blood work, among others. The dosage can be determined by a physician and adjusted, as necessary, to suit observed effects of the treatment.

### Dosage

“Unit dosage form” as the term is used herein refers to a dosage for suitable one administration. By way of example a unit dosage form can be an amount of therapeutic disposed in a delivery device, e.g., a syringe or intravenous drip bag. In one embodiment, a unit dosage form is administered in a single administration. In another, embodiment more than one-unit dosage form can be administered simultaneously.

The dosage of the agent as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to administer further cells, discontinue treatment, resume treatment, or make other alterations to the treatment regimen. The dosage should not be so large as to cause adverse side effects, such as cytokine release syndrome. Generally, the dosage will vary with the age, condition, and sex of the patient and can be determined by one of skill in the art. The dosage can also be adjusted by the individual physician in the event of any complication.

### Combinatorial Therapy

In one embodiment, the agent or compositions described herein is used as a monotherapy. In one embodiment, the agents described herein can be used in combination with other known agents and therapies for an inflammatory disease. Administered “in combination,” as used herein, means that two (or more) different treatments are delivered to the subject during the course of the subject's affliction with the disorder, e.g., the two or more treatments are delivered after the subject has been diagnosed with the disorder (an inflammatory disease) and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered. The agents described herein and the at least one additional therapy can be administered simultaneously, in the same or in separate compositions, or sequentially. For sequential administration, the agent described herein can be administered first, and the additional agent can be administered second, or the order of administration can be reversed. The agent and/or other therapeutic agents, procedures or modalities can be administered during periods of active disorder, or during a period of remission or less active disease. The agent can be administered before another treatment, concurrently with the treatment, post-treatment, or during remission of the disorder.

Therapeutics currently used to treat an inflammatory disease include but are not limited to non-steroidal anti-inflammatory drugs (e.g. acetaminophen, aspririn, ibuprofen, naproxen), tolmetin, ketorolac, diclofenac, enolic acids (e.g. piroxicam, meloxicam, nubumetone), pyrazolon derivatives (e.g. phenylbutazone), cyclooxygenase-2 selective inhibitors (e.g. rofecoxib, parecoxib, celecoxib, lumaricoxib, etoricoxib), apazone, gold, steroids (e.g. cortisone, prednisolone, methylprednisolone), disease-modifying antirheumatic drugs (e.g. anti-TNFα, anti-cyclic citrullinated peptide). Inflammatory diseases can further be a result of microbial or viral infections. Therapeutics to treat infectious diseases include antivirals (e.g. lopinavir, indinavir, nelfinavir, amprenavir, ritonavir, elvitegravir, dolutegravir, maraviro, enfuviritide, pleconaril, amantadine, rimantadine), antimicrobial agents (penicillin, ampicillin, amoxicillin, ciprofloxacin, levofloxacin, vancomycin, cycloserine, bacitracin, gentamycin, cefotetan, cefprozil), and anti-fungals (e.g. nystatin, amphotericin B).

It is contemplated that the methods described herein can be applied to immunotherapies for the treatment of cancer as the same drugs are often used for the treatment of autoimmune diseases (e.g. rheumatoid arthritis or organ transplants). Exemplary therapeutics used to treat cancer include, but are not limited to, immunotherapies such as dendritic cell vaccines, cell therapies, antibodies, interferons, or classical chemotherapeutic agent such as methotrexate, cyclophosphamide, 5-azacytadine, azathioprine, trimetrexate, leucovorin, vinblastine, bleomycin, doxorubicin, daunorunbicin, bortezomib, 5-fluorouracil, mitomycin, cisplatin, temozolamide, melphalen, ifosfamide, thiotepa, busulfan, carmustine, carboplatin, and any other chemotherapeutic known in the art.

When administered in combination, the agent or composition and the additional agent (e.g., second or third agent), or all, can be administered in an amount or dose that is higher, lower or the same as the amount or dosage of each agent used individually, e.g., as a monotherapy. In certain embodiments, the administered amount or dosage of the agent, the additional agent (e.g., second or third agent), or all, is lower (e.g., at least 20%, at least 30%, at least 40%, or at least 50%) than the amount or dosage of each agent used individually. In other embodiments, the amount or dosage of agent, the additional agent (e.g., second or third agent), or all, that results in a desired effect (e.g., treatment of an inflammatory disease) is lower (e.g., at least 20%, at least 30%, at least 40%, or at least 50% lower) than the amount or dosage of each agent individually required to achieve the same therapeutic effect.

### Parenteral Dosage Forms

Parenteral dosage forms of an agents described herein can be administered to a subject by various routes, including, but not limited to, subcutaneous, intravenous (including bolus injection), intramuscular, and intraarterial. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, controlled-release parenteral dosage forms, and emulsions.

Suitable vehicles that can be used to provide parenteral dosage forms of the disclosure are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate.

### Controlled and Delay Dosage Forms

In some embodiments of the aspects described herein, an agent or composition is administered to a subject by controlled- or delayed-release means. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. (Kim, Cherng-ju, Controlled Release Dosage Form Design, 2 (Technomic Publishing, Lancaster, Pa.: 2000)). Controlled-release formulations can be used to control a compound of formula (I)'s onset of action, duration of action, plasma levels within the therapeutic window, and peak blood levels. In particular, controlled- or extended-release dosage forms or formulations can be used to ensure that the maximum effectiveness of an agent is achieved while minimizing potential adverse effects and safety concerns, which can occur both from under-dosing a drug (i.e., going below the minimum therapeutic levels) as well as exceeding the toxicity level for the drug.

A variety of known controlled- or extended-release dosage forms, formulations, and devices can be adapted for use with any agent described herein. Examples include, but are not limited to, those described in U.S. Pat. Nos.: 3,845,770; 3,916,899; 3,536,809; 3,598,123; 4,008,719; 5674,533; 5,059,595; 5,591 ,767; 5,120,548; 5,073,543; 5,639,476; 5,354,556; 5,733,566; and 6,365,185, each of which is incorporated herein by reference in their entireties. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), multilayer coatings, microparticles, liposomes, or microspheres or a combination thereof to provide the desired release profile in varying proportions. Additionally, ion exchange materials can be used to prepare immobilized, adsorbed salt forms of the disclosed compounds and thus effect controlled delivery of the drug. Examples of specific anion exchangers include, but are not limited to, DUOLITE® A568 and DUOLITE® AP143 (Rohm&Haas, Spring House, Pa. USA).

### Efficacy

The efficacy of an agents described herein, e.g., for the treatment of an inflammatory disease, can be determined by the skilled practitioner. However, a treatment is considered “effective treatment,” as the term is used herein, if one or more of the signs or symptoms of the inflammatory disease are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate, e.g., fever, fatigue, pain, shortness of breath, etc. Efficacy can also be measured by a failure of an individual to worsen as assessed by hospitalization, or need for medical interventions (i.e., progression of the symptoms). Methods of measuring these indicators are known to those of skill in the art and/or are described herein.

Efficacy can be assessed in animal models of a condition described herein, for example, a mouse model or an appropriate animal model of an inflammatory disease, for example, endotoxemia, as the case may be. When using an experimental animal model, efficacy of treatment is evidenced when a statistically significant change in a marker is observed, e.g., macrophage activation, temperature, blood cell count, tumor size, or tumor mass.

The present invention can be defined in any of the following numbered paragraphs:
1. A method of treating an inflammatory condition in a subject, comprising: administering a modulation agent to inhibit the expression of Inc-FAM164A1.
2. The method of paragraph 1, wherein the modulation agent of expression of lnc-FAM164A1 is a nucleic acid molecule, small molecule, protein, polysaccharide, or antibody.
3. The method of any preceding paragraph, wherein the nucleic acid molecule is an antisense RNA, an RNA interference (RNAi) molecule, an RNA decoy molecule, or an RNAaptamer.
4. The method of any preceding paragraph, wherein the RNAi molecule is wherein the RNAi molecule is an siRNA molecule, an esiRNA molecule, an shRNA molecule, or a miRNA molecule.
5. The method of any preceding paragraph, wherein the protein is a peptide, polypeptide, or glycosylated protein.
6. The method of any preceding paragraph, wherein the inflammatory condition is characterized by activation of immune cells.
7. The method of any preceding paragraph, wherein the activation of immune cells is acute or chronic.
8. The method of any preceding paragraph, wherein immune cells include macrophages, T cells, dendritic cells, B cells, or neutrophils.
9. The method of any preceding paragraph, wherein the activation of immune cells is macrophage activation.
10. The method of any preceding paragraph, wherein the macrophage activation is characterized by uncontrolled macrophage activation and sustained inflammation.
11. The method of any preceding paragraph, wherein the inflammatory condition includes atherosclerotic vascular disease, heart valve disease, heart failure, autoimmune disease, osteoporosis, ectopic calcification, brain damage after stroke, obesity, fatty liver disease, diabetes, Gaucher's disease, sepsis, kidney dysfunction, kidney failure, and cancer.
12. The method of any preceding paragraph, wherein the atherosclerotic vascular disease is coronary artery disease, stent restenosis, carotid metabolic disease, stroke, acute myocardial infarction, heart failure, peripheral arterial disease, limb ischemia, vein graft failure, AV fistula failure.
13. The method of any preceding paragraph, wherein the heart valve disease is aortic or mitral valve disease.
14. The method of any preceding paragraph, wherein the inflammatory condition arises in artificial or tissue engineered tissues.
17. The method of any preceding paragraph, wherein the tissues are organs.
18. The method of any preceding paragraph, wherein the tissues are blood vessels or valves.
19. A method of modulating immune activation in cells, comprising administering to cells an effective amount of a modulation agent of expression of Inc-FAM164A1 in the cell.
20. The method of any preceding paragraph, wherein the effective amount of a modulation agent inhibits the expression of lnc-FAM164A1.
21. The method of any preceding paragraph, wherein the modulation agent of expression of lnc-FAM164A1 is a nucleic acid molecule, small molecule, protein, polysaccharide, or antibody.
22. The method of any preceding paragraph, wherein the nucleic acid molecule is an antisense RNA, an RNA interference (RNAi) molecule, a decoy molecule, or an RNAaptamer.
23. The method of any preceding paragraph, wherein the RNAi molecule is wherein the RNAi molecule is an siRNA molecule, an esiRNA molecule, an shRNA molecule, or a miRNA molecule.
24. The method of any preceding paragraph, wherein the protein is a peptide, polypeptide, or glycosylated protein.
25. The method of any preceding paragraph, wherein the protein modulation agent is ATP-citrate synthase (ACLY), filaggrin-2 (FLG2), or splicing factor, proline- and glutamine-rich (SFPQ).
26. The method of any preceding paragraph, wherein the protein modulating agent interacts with lnc-FAM164A1 as an interacting partner to enhance the activity of lnc-FAM164A1.
27. The method of any preceding paragraph, wherein the immune activation in cells is acute or chronic.
28. The method of any preceding paragraph, wherein cells include macrophages, T cells, dendritic cells, B cells, or neutrophils.
29. The method of any preceding paragraph, wherein the macrophages are activated macrophages.
30. The method of any preceding paragraph, wherein modulating immune activation in cells comprises a cell in a subject having an inflammatory condition.
31. The method of any preceding paragraph, wherein the inflammatory condition includes atherosclerotic vascular disease, heart valve disease, heart failure, autoimmune disease, osteoporosis, ectopic calcification, brain damage after stroke, obesity, fatty liver disease, diabetes, Gaucher's disease, sepsis, kidney dysfunction, kidney failure, and cancer.
32. The method of any preceding paragraph, wherein the atherosclerotic vascular disease is coronary artery disease, stent restenosis, carotid metabolic disease, stroke, acute myocardial infarction, heart failure, peripheral arterial disease, limb ischemia, vein graft failure, AV fistula failure.
33. The method of any preceding paragraph, wherein the heart valve disease is aortic or mitral valve disease.
34. The method of any preceding paragraph, wherein the inflammatory condition arises in artificial or tissue engineered tissues.
35. The method of any preceding paragraph, wherein the tissues are organs.
36. The method of any preceding paragraph, wherein the tissues are blood vessels or valves.
37. A pharmaceutical composition comprising a modulation agent to inhibit the expression of lnc-FAM164A1.
38. The composition of any preceding paragraph, wherein the modulation agent of expression of lnc-FAM164A1 is a nucleic acid molecule, small molecule, protein, polysaccharide, or antibody.
39. The composition of any preceding paragraph, wherein the nucleic acid molecule is an antisense RNA, an RNA interference (RNAi) molecule, a decoy molecule, or an RNAaptamer.
40. The composition of any preceding paragraph, wherein the RNAi molecule is wherein the RNAi molecule is an siRNA molecule, an esiRNA molecule, an shRNA molecule, or a miRNA molecule.
41. The method of any preceding paragraph, wherein the protein is a peptide, polypeptide, or glycosylated protein.
42. The method of any preceding paragraph, wherein the protein modulation agent is ATP-citrate synthase (ACLY), filaggrin-2 (FLG2), or splicing factor, proline- and glutamine-rich (SFPQ).
43. The composition of any preceding paragraph, wherein the protein modulating agent interacts with lnc-FAM164A1 as an interacting partner to enhance the activity of lnc-FAM164A1.
44. The composition of any preceding paragraph, wherein the modulating agent is in a pharmaceutically acceptable formulation.
45. The composition of any preceding paragraph, wherein the modulation agent is administered to a subject having an inflammatory condition
46. The method of any preceding paragraph, wherein the inflammatory condition includes atherosclerotic vascular disease, heart valve disease, heart failure, autoimmune disease, osteoporosis, ectopic calcification, brain damage after stroke, obesity, fatty liver disease, diabetes, Gaucher's disease, sepsis, kidney dysfunction, kidney failure, and cancer.
47. The method of any preceding paragraph, wherein the atherosclerotic vascular disease is coronary artery disease, stent restenosis, carotid metabolic disease, stroke, acute myocardial infarction, heart failure, peripheral arterial disease, limb ischemia, vein graft failure, AV fistula failure.

MGH 24231 draft paragraphs
48. The method of any preceding paragraph, wherein the heart valve disease is aortic or mitral valve disease.
49. The method of any preceding paragraph, wherein the inflammatory condition arises in artificial or tissue engineered tissues.
50. The method of any preceding paragraph, wherein the tissues are organs.
51. The method of any preceding paragraph, wherein the tissues are blood vessels or valves.

The present invention can be further defined in any of the following numbered paragraphs:
1. A method of treating or preventing an inflammatory disease in a subject, the method comprising: administering to a subject an effective amount of an agent that inhibits a long noncoding RNA(lnc-RNA) expressed in a macrophage.
2. The method of paragraph 1, wherein the lnc-RNA is selected from the group consisting of: AL078621.3; RP11-536K7.5; RP11-143M1.3; RP11-561O23.5; AC10980.2; P2RX7; LOC440896; D63785; RP11-58A12.2; RP11-79H23.3 (lnc-FAM164A1); and RP13-452N2.1.
3. A method of treating or preventing an inflammatory disease in a subject, the method comprising: administering to a subject an effective amount of an agent that inhibits lnc-FAM164A1.
4. The method of any preceding paragraph, wherein the inflammatory disease is selected from the group consisting of: endotoxemia, atherosclerotic vascular disease, coronary artery disease, peripheral arteru disease, in-stent restenosis, vein graft disease, arteriovenous fistula disease, vascular calcification, heart valve disease, heart failure, autoimmune disease, osteoporosis, ectopic calcification, brain damage after stroke, obesity, fatty liver disease, diabetes, Gaucher's disease, sepsis, kidney dysfunction, kidney failure, and cancer.
5. The method of any preceding paragraph, wherein the heart valve disease is aortic valve disease or mitral valve disease.
6. The method of any preceding paragraph, wherein the inflammatory disease occurs in response to a tissue or cell transplantation.
7. The method of any preceding paragraph, wherein the inflammatory disease is acute or chronic.
8. The method of any preceding paragraph, wherein the agent is selected from the group consisting of: a small molecule, an antibody, a peptide, a genome editing system, an antisense oligonucleotide, and an RNA interference (RNAi), an antisense RNA, an RNA decoy molecule, an RNAaptamer, and an inhibitory polypeptide.
9. The method of any preceding paragraph, wherein the RNAi is microRNA, an siRNA, or a shRNA
10. The method of any preceding paragraph, wherein the agent is a vector that encodes the agent.
11. The method of any preceding paragraph, wherein the vector is non-integrative or integrative.
12. The method of any preceding paragraph, wherein the non-integrative vector is selected from the group consisting of an EBNA1 vector, a minicircle vector, a non-integrative adenovirus, a non-integrative RNA, and a Sendai virus.
13. The method of any preceding paragraph, wherein the vector is an episomal vector.
14. The method of any preceding paragraph, wherein the vector is a lentiviral vector.
15. The method of any preceding paragraph, wherein the lnc-RNA is inhibited in a target cell.
16. The method of any preceding paragraph, wherein lnc-FAM164A1 is inhibited in a target cell.
17. The method of any preceding paragraph, wherein the target cell is a mammalian cell.
18. The method of any preceding paragraph, wherein the target cell is a macrophage, T cell, dendritic cells, B cell, natural killer cell, or neutrophil.
19. The method of any preceding paragraph, wherein the macrophage is an activated macrophage.
20. The method of any preceding paragraph, wherein the subject is a mammal.
21. The method of any preceding paragraph, wherein the subject is a human.
22. The method of any preceding paragraph, wherein inhibiting is reducing the level and/or activity.
23. The method of any preceding paragraph, wherein the effective amount of the agent reduces the level of lnc-RNA by at least 50%, at least 60%, at least 70%, at least 80%, at least 90%, or more as compared to an appropriate control.
24. The method of any preceding paragraph, wherein the effective amount of the agent reduces the activity of lnc-RNA by at least 50%, at least 60%, at least 70%, at least 80%, at least 90%, or more as compared to an appropriate control.
25. The method of any preceding paragraph, wherein the effective amount of the agent reduces the level of lnc-FAM164A1 by at least 50%, at least 60%, at least 70%, at least 80%, at least 90%, or more as compared to an appropriate control.
26. The method of any preceding paragraph, wherein the effective amount of the agent reduces the expression of lnc-FAM164A1 by at least 50%, at least 60%, at least 70%, at least 80%, at least 90%, or more as compared to an appropriate control.
27. The method of any preceding paragraph, wherein the subject has previously been diagnosised with having an inflammatory disease.
28. The method of any preceding paragraph, wherein the subject has not previously been diagnosed with having an inflammatory disease.
29. The method of any preceding paragraph, wherein the subject exhibits at least one risk factor of developing an inflammatory disease.
30. The method of any preceding paragraph, further comprising, prior to adminisitering, diagnosing a subject as having an inflammatory disease.
31. The method of any preceding paragraph, further comprising, prior to administering, receiving the results of an assay that diagnoses a patient as having an inflammatory disease.
32. The method of any preceding paragraph, wherein the agent decreases expression of CCL2 and IL-6 in a plasma cell and/or a peritoneal cell.
33. The method of any preceding paragraph, wherein the agent decreases NFkB signaling.
34. A method of treating or preventing an inflammatory disease in a subject, the method comprising: administering to a subject an effective amount of an agent that inhibits a protein bound to a lnc-RNA.
35. The method of any preceding paragraph, wherein the protein bound to the lnc-RNA is selected from the group consisting of: ATP-citrate synthase (ACLY); Filaggrin-2 (Flg2); and Splicing factor, proline-and-glutamine-rich (SFPQ).
36. The method of any preceding paragraph, wherein the protein bound to the lnc-RNA is ACLY.
37. The method of any of any preceding paragraph, wherein the agent decreases the expression of CCL2 and TNF-α in the subject.
38. The method of any of any preceding paragraph, wherein inhibiting reduces the level or activity of the protein.
39. The method of any of any preceding paragraph, wherein inhibiting reduces the binding of the protein to the lnc-RNA.
40. The method of any preceding paragraph, wherein the lnc-RNA is lnc-FAM164A1.
41. The method of any preceding paragraph, wherein the effective amount of the agent reduces the level of the protein by at least 50%, at least 60%, at least 70%, at least 80%, at least 90%, or more as compared to an appropriate control.
42. The method of any preceding paragraph, wherein the effective amount of the agent reduces the activity of the protein by at least 50%, at least 60%, at least 70%, at least 80%, at least 90%, or more as compared to an appropriate control.
43. The method of any preceding paragraph, wherein the effective amount of the agent reduces the binding of the protein to the lnc-RNA by at least 50%, at least 60%, at least 70%, at least 80%, at least 90%, or more as compared to an appropriate control.
44. A method of treating an inflammatory disease in a subject, the method comprising:
   a. receiving results of an assay that measures a level of a lnc-RNA expressed in a macrophage in a biological sample;
   b. comparing the level of the lnc-RNA to a reference level;
   c. identifying a subject as having an inflammatory disease if the level of the lnc-RNA is increased as compared to the reference level; and
   d. administering to the subject having an inflammatory disease an agent that inhibits the lnc-RNA, or a composition comprising an agent that inhibits the lnc-RNA.
45. A method of treating an inflammatory disease in a subject, the method comprising:
   a. measuring a level of a lnc-RNA expressed in a macrophage in a biological sample;
   b. comparing the level of the lnc-RNA to a reference level;
   c. identifying a subject as having an inflammatory disease if the level of the lnc-RNA is increased as compared to the reference level; and
   d. administering to the subject having an inflammatory disease an agent that inhibits the lnc-RNA, or a composition comprising an agent that inhibits the lnc-RNA.
46. The method of any preceding paragraph, wherein the lnc-RNA is selected from the group consisting of: AL078621.3; RP11-536K7.5; RP11-143M1.3; RP11-561O23.5; AC10980.2; P2RX7; LOC440896; D63785; RP11-58A12.2; RP11-79H23.3 (lnc-FAM164A1); and RP13-452N2.1.
47. The method of any preceding paragraph, wherein the lnc-RNA is lnc-FAM164A1.
48. The method of any preceding paragraph, further comprising, prior to step a), obtaining a biological sample from the subject.
49. A pharmaceutical composition comprising an agent that inhibits a lnc-RNA expressed in a macrophage and a pharmaceutically acceptable carrier.
50. A pharmaceutical composition comprising an agent that inhibits lnc-FAM164A1 and a pharmaceutically acceptable carrier.
51. A pharmaceutical composition comprising an agent that inhibits a protein bound to a lnc-RNA and a pharmaceutically acceptable carrier.
52. The pharmaceutical composition of any preceding paragraph, wherein the protein bound to the lnc-RNA is selected from the group consisting of: ATP-citrate synthase (ACLY); Filaggrin-2 (Flg2); and Splicing factor, proline-and-glutamine-rich (SFPQ).
53. The pharmaceutical composition of any preceding paragraph, wherein the agent is selected from the group consisting of: small molecule, an antibody, a peptide, a genome editing system, an antisense oligonucleotide, and an RNA interference (RNAi), an antisense RNA, an RNA decoy molecule, an RNAaptamer, or an inhibitory polypeptide.
54. The pharmaceutical composition of any preceding paragraph, wherein the RNAi is microRNA, an siRNA, or a shRNA.
55. A method of identifying a subject having sepsis, the method comprising:
   a. measuring a level of a lnc-FAM164A1 in a biological sample;
   b. comparing the level of the lnc-FAM164A1 to a reference level;
   c. identifying a subject as having sepsis if the level of the lnc-RNA is increased as compared to the reference level; and
   d. administering to the subject having sepsis a therapeutic to treat sepsis.
56. The method of any preceding paragraph, further comprising, prior to step (a), obtaining a biological sample from the subject.
57. The method of any preceding paragraph, wherein the biological sample is a blood sample.

### EXAMPLES

### Example 1

Introduction

Long noncoding RNAs (lnc-RNAs, >200nt), non protein coding transcripts known as the “dark matter” of the genome, play unsuspected roles in numerous biological processes and human diseases including embryonal development, tumor growth, metastasis, cardiometabolic disorders, and inflammation. Based on the location to their neighboring protein-coding genes, lnc-RNAs are classified as antisense, intronic, divergent and intergenic lnc-RNAs.¹ Although relatively less evolutionary conservation than protein coding genes,² intergenic lnc-RNAs can regulate gene transcription in a tissue-specific, cell type-specific, or disease-stage-dependent manner.³⁻⁵ Several lnc-RNAs have been characterized as important regulators among the differentiation of immune cells, such as erythroid cells, T-lymphocytes, dendritic cells and monocytes/macrophages.⁶⁻⁹ ENREF 5 Their biological function in immunity, however, remains largely undetermined.

Macrophages are large phagocytes and a key player in innate immunity. Due to their ability to engulf pathogens, lipids, apoptotic cells, damaged tissue and to interplay with other immune cells, macrophages play an essential role in host defense and also contribute to the pathogenesis of various inflammatory diseases. Macrophages are remarkably plastic and can switch phenotype depending on the cellular stimulus in the tissue microenvironment. Alternatively, the balance of macrophage subpopulations may shift in response to molecular cues, determining the inflammatory state of normal or pathological tissues. ¹⁰⁻¹³ In vitro, cultured macrophages can be skewed into a pro-inflammatory phenotype using bacterial LPS or IFN-γ or an anti-inflammatory phenotype induced by IL-4 or IL-13. LPS recognizes Toll-like receptor 4 (TLR4) and activates macrophages. LPS-induced signaling triggers the phosphorylation and ubiquitin-dependent degradation of IκB proteins, resulting in the translocation of NF-κB dimers (e.g. p65/p50) from cytoplasm to nucleus and the production of pro-inflammatory mediators (e.g. IL-1β, IL-6, TNF-α).

Although human and murine macrophages express thousands of lnc-RNAs, limited numbers of lnc-RNAs have been characterized in modulating the activation of macrophages.¹⁵⁻²⁹ Only several lnc-RNAs have conserved expression in both human and mouse macrophages.²⁶⁻²⁸ It was first reported that ligation of pattern recognition receptors (e.g. TLRs) increases the expression of long intergenic noncoding RNA (lincRNA)-Cox2, located proximally to the PG-endoperoxide synthase 2 (Ptgs2/Cox2).¹⁵ LincRNA-Cox2 plays dual roles in modulating the expression of immune response genes in murine bone marrow-derived macrophages, which are mediated by its interaction with heterogeneous nuclear ribonucleoprotein (hnRNP) AB and A2/B1, modulation of Mi-2/NuRD-mediated epigenetic histone modifications as well as SWI/SNF-associated chromatin remodeling.¹⁵¹⁷ In addition, several other mouse lnc-RNAs have been identified as modulators of inflammatory gene expression in macrophages and in mouse models of diabetes, atherosclerosis and endotoxemia.^{18-21,25} Discovered in the human macrophage-like cell line THP-1, lnc-RNA-THRIL (Linc1992) specifically interacts with hnRNP-L and regulates the transcription of TNF-α cytokine induced by TLR2 agonist (Pam3CSK4).²² Two recent reports demonstrated the increased expression of a late-response lnc-RNA-TCONS 00019715 and lnc-MacORIS in human primary macrophages elicited by LPS and IFN-γ, which regulate macrophage polarization and IFN-γ/JAK2/STAT1 signaling pathway.^{23,29}

To identify intergenic lnc-RNAs that regulate macrophage activation in inflammatory diseases, lnc-RNA microarray analysis of human PBMC-derived macrophages activated by LPS for 3 hours was performed. Unbiased bioinformatic analysis selected 11 lnc-RNAs that significantly increased by LPS stimulation. Intergenic lnc-RNA-FAM164A1-1 (lnc-FAM164A1) that locates close to the protein coding gene FAM164A (family with sequence similarity 164, member A) was selected for validation studies of the systems approach described herein. Mechanistic studies revealed that human lnc-FAM164A1 enhances the expression of pro-inflammatory molecules in part through NF-κB signaling in LPS-activated macrophages.

Methods and Materials

Reagents. Lymphocyte separation medium (LSM) was obtained from MP Biomedicals. Ultrapure LPS (tlrl-smlps, from S. minnesota R595) was obtained from InvivoGen. Bayl1-7082 was obtained from Sigma-Aldrich. Antibodies used in western blots were as follows: mouse monoclonal antibodies against β-actin (Novus Biologicals, NB600-501) and lamin A/C (Active motif, 39288), rabbit antibodies against p65 (Santa Cruz Biotechnology, sc-372), against IκB-α (Cell Signaling, 9242), against ACLY (Abcam, ab40793) and against hn RNP A1(ThermoFisher Scientific, PAS-19431). pGL4.32[1uc2P/NF-κB-RE/Hygro] Vector was purchase from Promega (E8491) and Dual-Glo Luciferase Assay System was from VWR (PAE2920). Lipofectamine® 3000 Reagent (L3000015) was from Life Technologies. Pierce RIPA Buffer (PI-89900), Pierce IP Lysis Buffer (87787), Pierce™ RNA 3′ End Desthiobiotinylation Kit, Pierce™ Magnetic RNA-Protein Pull-Down Kit, and Halt Combined Protease and Phosphatase Inhibitor Cocktails (PI-78441) were from ThermoFisher Scientific.

Cell cultures. THP-1 cells. The human monocytic leukemia cell line (THP-1) was purchased from ATCC (TIB-202) and cultured with Roswell Park Memorial Institute (RPMI) 1640 medium supplemented with 10% FBS, penicillin/streptomycin in cell culture incubator at 37° C. (5% CO2). THP-1 cells were plated in 12-well plates at 1.0×10′ per well and differentiated into macrophage-like cells by the addition of 100 ng/mL of PMA (Sigma-Aldrich P8139) for 48 hours.

Human primary macrophages derived from peripheral blood mononuclear cells (PBMC). PBMCs were isolated from blood buffy coat (Research Blood Components, Brighton, MA) using lymphocyte separation medium (LSM) as described previously.³⁰ PBMCs (5×10⁶ cells/well) were plated in 6-well culture plates and maintained in RPMI-1640 supplemented with 5% human serum and penicillin/streptomycin at 37° C(5% CO₂) for 7-10 days before use.

Mouse peritoneal macrophages. Peritoneal macrophages were harvested 4 days after intraperitoneal injection of 2.5 ml of 4% Brewer thioglycollate medium (BD Diagnostic Systems, Sparks, Md.) into C57BL/6J mice (8-10 weeks old, male, Jackson Laboratory). ³¹ 1×10⁶ cells were cultured on 12-well plates (Corning) with RPMI 1640 medium supplemented with 10% FBS and antibiotics (penicillin, streptomycin and amphotericin B). Non-adherent cells were washed with PBS after 1 hour culture.

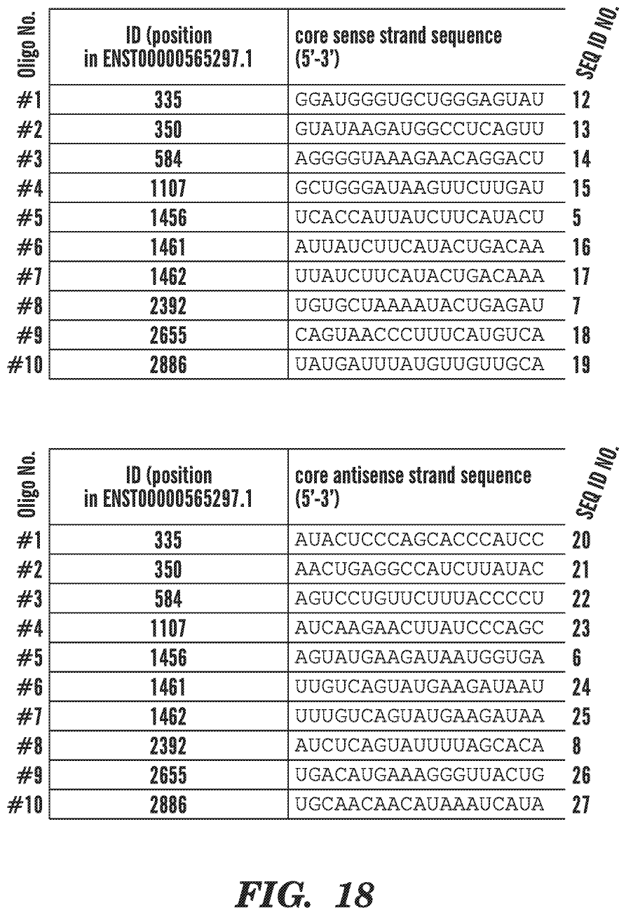

Silencing of lnc-FAM164A1 by antisense and si-RNA oligonucleotides on human PBMC-derived macrophages. Lnc-FAM164A1 RNA silencing was performed on human PBMC-derived macrophagesas using SilenceMag (BOCA Scientific, Boca Raton, Fla.) at final concentration of 50-100 nM.32 36-48 hours after transfection, macrophages were stimulated by 10 ng/mL LPS for 2-6 hours before collection of macrophages and cell culture medium. Anti-sense LNATM long RNA GepmeR oligonucleotides target lnc-FAM164A1 (Sequence 5′-GGTAAGAAATAGGTTG-3′(SEQ ID NO: 52)) and nonspecific negative control oligonucleotides (Sequence 5′-AACACGTCTATACGC-3′(SEQ ID NO: 53)) were designed by Exiqon A/S (Vedbaek, Denmark). Si-RNA Oligonucleotides targeting lnc-FAM164A1 and luciferase control siRNA oligos were designed by Axolabs (Kulmback, Germany). Their sequences and chemical modifications are as follows (A, G, U, C: RNA nucleotide; a, g, u, c: 2′-O-methyl-nucleotide, s: Phosphorothioate, dT: desoxy-T residue): Luciferase control siRNA (XD-00194): Sense strand: 5′-cuuAcGcuGAGuAcuucGAdTsdT-3′ (SEQ ID NO: 54); Antisense strand: 5′-UCGAAGuACUcAGCGuAAGdTsdT-3′ (SEQ ID NO: 4). Lnc-FAM164A1 specific siRNA oligo#5 (XD-05325): 5′-ucAccAuuAucuucAuAcudTsdT-3′(SEQ ID NO: 5) Antisense strand: 5′-AGuAUGAAGAuAAUGGUGAdTsdT-3′ (SEQ ID NO: 6). Lnc-FAM164A1 specific siRNA oligo#8 (XD-05328): 5′-uguGcuAAAAuAcuGAGAudTsdT-3′ (SEQ ID NO: 7); Antisense strand: 5′-AUCUcAGuAUUUuAGcAcAdTsdT-3′ (SEQ ID NO: 8). Additional oligos used to inhibit lnc-FAM164A1 are shown in FIG. 18.

RNA extraction and RT-PCR. Total RNA were extracted using an Illustra RNAspin Mini kit (GE Healthcare, Piscataway, N.J.) and the genomic DNA was removed by on-column DNase I digestion at RT for 15 minutes. cDNAs were synthesized using a high capacity cDNA reverse transcription kit (Applied Biosystems, Carlsbad, Calif.). Real-time PCR was performed on a 7900HT fast real-time PCR system (Applied Biosystems). Taqman probe and primers for lnc-FAM164A1 were designed by Integrated DNA Technologies and listed as follows: Probe: 5′-/56-FAM/ATGG CCTCA/ZEN/GTTAGGACTGCTTGG/3IABkFQ/-3′ (SEQ ID NO: 9); Primer1: 5′-GATGGGTGC TGGGAGTATAAG-3′ (SEQ ID NO: 10); Primer2: 5′-CCGCTCTCTTTCTCTCTGTTAG-3′ (SEQ ID NO: 11). Other Taqman probes for IL-1β, IL-6, IL-10, CCL2, TNF-α, and GAPDH was purchased from Life Technologies. Relative expression of each gene was normalized by GAPDH.

In Situ Hybridization using RNAscope.

Lnc-FAM164A1 RNA in situ was detected using RNAscope 2.5 Red Chromogenic assay (Advanced Cell Diagnostics, Inc., Catalog 322360) according to the manufacturer's manual. The 20ZZ probe named Hs-lnc-FAM164A-1 (Catalog 475881) was designed to detect the 412-1623 of lnc-FAM164A-1:1. Briefly, human PBMC-derived macrophages were cultured on Millicell EZ slide (Millipore). After the stimulation with 10 ng/mL of LPS for certain hours, macrophage monolayers were fixed by 10% Neutral Buffered Formalin for 30 min at RT, and then treated with hydrogen peroxide and Protease III before performing the RNAscope assay. After RNAscope staining, macrophages were stained with mouse anti-human CD68 (clone PG-Ml, DAKO) at 4 degree overnight and FITC-labelled goat anti-mouse IgG at RT for 1 hour. Slides were added mounting medium with DAPI (VECTASHIELD) (Vector Laboratory, Burlingame, Calif., USA) before placing coverslips. Slides were examined using the confocal microscope Al (Nikon) and the images were processed with Elements 3.20 software (Nikon).

ELISA. Duoset ELISA kits (R&D systems, Minneapolis, Minn.) were used to detect the levels of cytokines (human and mouse CCL2, IL-6, TNF-α) in culture medium from macrophages or mouse plasma.

Lnc-FAM164A1 RNA pulldown assay. Lnc-FAM164A1 sense and antisense RNA were in vitro transcribed with T7 RNA polymerase (Invitrogen) and purified by RNA Clean & Concentrator™-5 (Zymo Research). RNA (10 pg) was labeled with biotin at 3′-terminus using Pierce™ RNA 3′ End Desthiobiotinylation Kit. The THP-1 differentiated macrophage lysates were prepared using Pierce IP Lysis Buffer supplemented with Anti-RNase and Protease/Phosphatase Inhibitor Cocktail. RNA pulldown was performed by using the Pierce™ Magnetic RNA-Protein Pull-Down Kit. Streptavidin Magnetic Beads were first prepared according to manufacturer's instructions and then immediately subjected to the capture of biotin-labeled RNA in RNA capture buffer for 30 minutes at RT with agitation. The RNA-captured beads were incubated with 200 pg cell lysates diluted in Protein-RNA Binding Buffer for 1 hour at 4° C. with rotation. The RNA-binding protein complexes were washed three times with washing buffer and then eluted by D-biotin Elution Buffer at 37° C. for 30 minutes. The eluted protein complexes were denatured, reduced, and separated with 7.5% Mini-PROTEAN TGX™ Precast Protein Gels (Bio-Rad) with a running of 25% of gel length. After the Bio-Safe Coomassie Stain (Bio-Rad), the gel contained stained proteins were collected, washed, alkylated and digested with trypsin at 37° C. overnight for MS analysis. The eluted proteins were denatured and reduced in SDS-sample buffer (Boston Bioproducts) and used for western blots.

Mass Spectrometry.The RNA pulldown samples were analyzed with the high resolution/accuracy Orbitrap Fusion Lumos mass spectrometer fronted with an EASY spray source, and coupled to an Easy-nLC1000 HPLC pump (Thermo Scientific). The peptides were separated using a dual column set-up: an Acclaim PepMap RSLC C18 trap column, 75 μm×20 mm; and an EASY spray LC heated (45° C.) column, 75 μm×250 mm. The gradient was run at 300 nL/min from 5-21% solvent B (acetonitrile/0.1% formic acid) for 50 minutes, 21-30% Solvent B for 10 minutes, followed by five minutes of 95% solvent B. Solvent A was 0.1% formic acid. The instrument was set at 120 K resolution, and the top N precursor ions in 3 seconds cycle time (within a scan range of 375-1500 m/z) were subjected to collision induced dissociation (collision energy 30%) for peptide sequencing (MS/MS).

Western blot. Macrophages whole cell lysate were prepared using RIPA buffer containing protease inhibitor (Roche). Total protein was separated by 4-20% Mini-PROTEAN® TGX™ Precast Gel and transferred using the iBlot Western blotting system (Life Technologies). Primary antibodies against IκB-α, p65, ACLY, LaminA/C and β-actin were used. Protein expression was detected using Pierce ECL Western Blotting substrate reagent (ThermoFisher Scientific) and ImageQuant LAS 4000 (GE Healthcare).

Recombinant adenoviruses and trasnfectoin on macrophages. Lnc-FAM164A1 cDNA was isolated from human PBMC-derived macrophages was amplified by PCR and then cloned into the pcDNA3.1 vector. Lnc-FAM164A1 oligonucleotides were first amplified from pcDNA3.1- lnc-FAM164A1, sub-cloned into the pENTR11 entry vector, and then transferred to the pAd-CMV-V5 adenoviral vector through LR recombination using Gateway LR Clonase II Enzyme Mix to generate pAd/CMV/lnc-FAM164A1 (Ad-lnc-FAM164A1) (Life Technologies, Grand Island, N.Y., USA). pAd/CMV/V5-GW/lacZ was used as control vector. ³³ Adenovirus was amplified by transfection of PacI-digested vector in HEK293A cells with subsequent purification using Fast Trap Adenovirus Purification and Concentration (EMD Millipore) and dialysis in 10 mM Tris (pH 7.4) buffer with 10% (v/v) glycerol, and storage at −70° C. Viral titer was determined by plaque assay on 293 cells using Adeno-XTM rapid titer kit (Clontech, Mountain View, Calif., USA) and expressed as infectious units (ifu). For the in vitro cultured primary macrophages and THP-1-differentiated cells, both Ad-lacZ and Ad-lnc-FAM164A1 virus were added into the cells at 1000 ifu/macrophages and incubated for 48 hours. After a medium change, macrophages were stimulated with 10 ng/mL LPS for 0-3 hours before extraction of protein and mRNA for western blot and RT-PCR, respectively.

NF-κB-luciferase reporter assay. HEK-293 cells were plated in 24-well plates (0.5×10⁵/well). On the next day, cells were co-transfected of pGL4.32 vector [1uc2p/ NF-κB-RE/Hygro] (Promega, E8491) with either pcDNA3.1 empty vector or pcDNA3.1-lnc-FAM164A1 using Lipofectamine 3000. 24 hours later, the cells were stimulated 10 ng/mL LPS for another 6h. Firefly luciferase activity was measured using Dual-Glo luciferase assay kit (Promega).

Mass spectrometric data analysis. All MS/MS data were queried against the human UniProt database (downloaded on August 01, 2014) using the HT-SEQUEST search algorithm, via the Proteome Discoverer (PD) Package (version 2.1, Thermo Scientific). Methionine oxidation was set as a variable modification and carbamidomethylation of cysteine residues was set as a fixed modification. Peptides were filtered based on a 1% FDR. Peptides assigned by Proteome Discoverer to a given protein group (Master Protein), and not present in any other protein group, were considered as unique and used for quantification. A minimum of two unique peptides were included. Quantification was done using the area-under-the-curve (AUC) of up to the top three most abundant peptides per protein.

Network analysis. To quantify the disease associations of the lnc-FAM164A1 interacting candidate proteins, ACLY, FLG2 and SFPQ, the average shortest network distance between their modules and disease-related proteins were measured, where network distance is defined as the non-Euclidean distance measured in terms of the number of edges between two nodes. ACLY, FLG2 and SFPQ modules are defined as the subgraphs consisting of the respective candidate protein and its first neighbors, i.e. direct interaction partners, in the interactome. The average shortest distance D of a candidate module to disease genes is measured by calculating the shortest distance between each candidate module gene s and all genes t of a disease and then averaging over all candidate module module genes s such that

$$D = \frac{1}{‖ S ‖} {\sum }_{s ∈ S} {d}_{s} and {d}_{s} = \frac{1}{‖ T ‖} {\sum }_{t ∈ T} {d}_{st} ,$$

where dₛₜ is the shortest distance between s and t and S and T are the sets of genes in the ACLY, FLG2 and SFPQ first neighbors modules and disease genes, respectively. To compare the average shortest distance value to random expectation, the average shortest distance of the same number of randomly selected genes to disease genes was calculated for N=250 realizations. To control for degree (i.e., the number of connections of a gene), the random selection was done in a degree-preserving manner where all genes were binned according to their degree and random genes were selected uniformly at random from their corresponding degree bin. Empirical p-values were calculated by pₑₘₚ=P(Dᵣ<D), where Dᵣ is the average shortest distance of the randomized instance. The interactome onto which the ACLY, FLG2 and SFPQ modules and disease genes were mapped consists of curated physical protein-protein interactions with experimental support, including binary interactions, protein complexes, enzyme-coupled reactions, signaling interactions, kinase-substrate pairs, regulatory interactions and manually curated interactions from literature, as described previously.' Disease genes were obtained from the DiseaseConnect (http://disease-connect.org)³⁵ (using entries with evidence from Genome-Wide Association Studies (GWAS) and Online Mendelian Inheritance in Man (OMIM) (e.g., available on the world wide web at www.omim.org/) and MalaCards (e.g., available on the world wide web at www.malacards.org/) ³⁶ databases.

Gain-of-function study by adenovirus in mouse model of endotoxemia. C57BL/6J mice (8-10 weeks old, male) were purchased from Jackson Laboratory. All animal experiments were approved by Institutional Animal Care and Use Committee at Beth Israel Deaconess Medical Center. 5×10⁹ ifu of Ad-lacZ or Ad-lnc-FAM164A1 virus (0.2 mL) were intravenously injected into the mice through tail vein. After 3 Days, mice were administrated with 4 mg/kg of LPS into peritoneum.³⁷ All mice were then euthanized 3 hours after LPS challenge. Whole blood was collected from Inferior Vena Cava (IVC) into sodium citrate (final concentration: 0.38%). Mouse plasma was prepared by spinning at 4000×g for 15 min. Tissue was collected or embedded in O.C.T. compound (Tissue-Tek) and then frozen in liquid nitrogen and stored at −80° C. until further use.

Humanised mouse model of endotoxemia. Hu-CD34 NSG mice or Hu-CD34 NSG-SGM3 mice (4-5 months old, female) were purchased from Jackson Laboratory. NSG (NOD scid gamma) mouse strain was served as the host and transplanted with human CD34+Hematopoietic stem cells (HSCs). Small amount of blood were collected from Hu-CD34 NSG mice through intraorbital bleeding. On next day, these Hu-CD34 NSG mice were administrated intraperitoneally with 5 mg/kg of LPS and then euthanized 6 hours after LPS challenge. In loss-of-function study, NSG-SGM3 mice were administrated of 0.5 mg/kg Lipid nanoparticle formulated si-RNA against lnc-FAM164A1 intravenously (twice in 5 days), followed by a bonus i.p. injection of 5 mg/kg LPS. Whole blood was collected from the IVC into sodium citrate. Mouse plasma was prepared by spinning at 4000×g for 15 min. Liver, spleen and lung tissues were collected and stored at −80 ° C. until further use. Total white blood cell (WBC) was prepared by using ACK (Ammonium-Chloride-Potassium) Lysing Buffer. Splenocytes were isolated as described previously. ³² After incubation with anti-mouse CD16/CD32 mAb (BioLegend) to block the Fc receptor, cells were then stained with Alexa Fluor 488-conjugated human CD45 APC-Cy7-conjugated anti-mouse CD45 (BioLegend) in autoMACS running buffer containing bovine serum albumin, EDTA, PBS and 0.09% azide (Miltenyl Biotee) for 30 minutes. Stained cells were analyzed by FACSAria2 (BD Bioscience) and Flowjo software.

Statistical analysis. Data are shown as mean±standard deviation (SD). And, “n” indicates the number of independent experiments or number of animals/samples. Differences between groups were analyzed using Student's t-test or one-way ANOVA with Dunn's Multiple Comparison Test (GraphPad prism 5, Prism Software Inc. La Jolla, Calif.). A probability value p<0.05 was considered to be statistically significant.

Results

Global microarray screening and unbiased bioinformatic analysis identifies lnc-FAM164A1 in LPS-activated human primary macrophages.

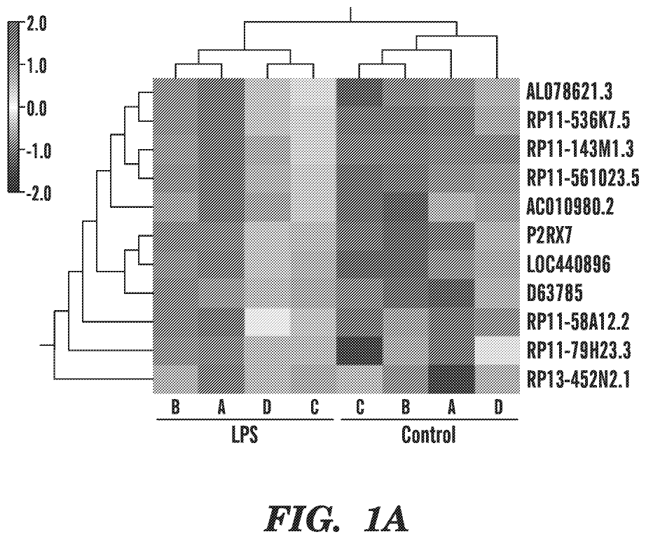

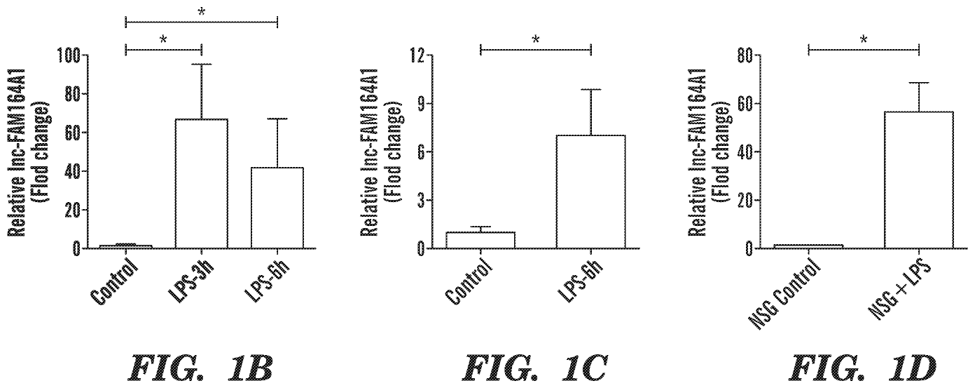

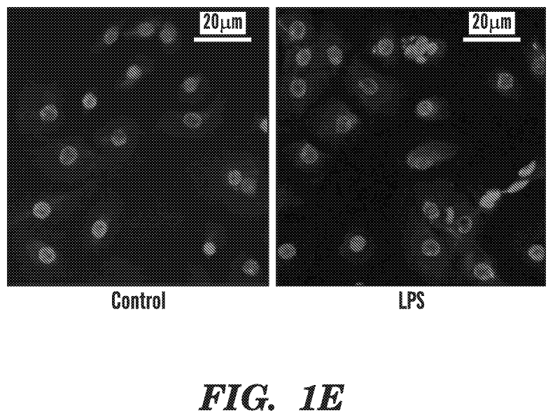

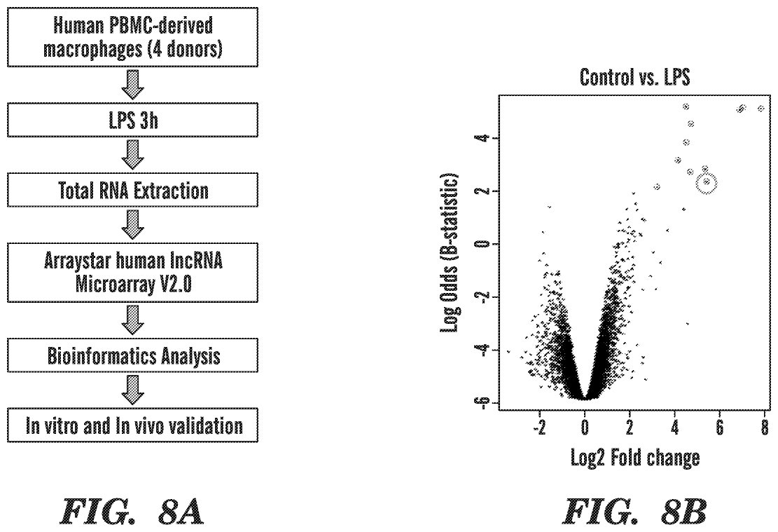

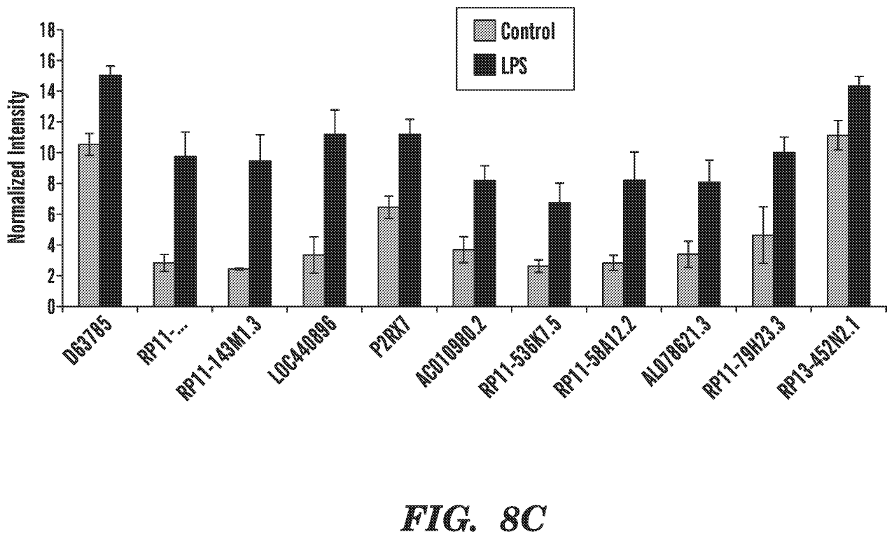

To identify potential key regulators of macrophage activation, early-responsive non protein coding transcripts in LPS-elicited human primary macrophages were explored. Human lnc-RNA microarray analysis was performed of RNA samples purified from PBMC-derived human primary macrophages, isolated from four different donors, 3 hours after LPS stimulation (FIG. 8A for the experimental design). The data underwent unbiased bioinformatics analysis by the “limma” R package.' LPS treatment produced statistically significant increases in the expression of 11 lnc-RNAs (adjusted p-value FDR<0.05 and Fold-change≥2) (FIG. 1A and FIG. 8C), as also shown in the volcano plot (FIG. 8B). These 11 lnc-RNAs candidates included 7 intergenic, 2 antisense, 1 intro-sense overlapping and 1 exon-sense overlapping lnc-RNAs. lnc-RNA RP11-79H23.3 was selected as an example to validate the systems approach, based on the following factors: 1) it is classified as intergenic lnc-RNA; 2) it has only one transcript of 2994 base pair; and 3) its biological function is unknown. It is annotated as Lnc-FAM164A-1(lnc-ZC2HC1A-1) in LNCipedia,³⁹ locates at chromosome 8 (positions 79749764 to 79752757) close to the protein coding gene FAM164A (family with sequence similarity 164, member A). Its alternative gene names include ENSG00000261618.1 and OTTHUMG00000173421.1. The coding potential of lnc-FAM164A-1 was analyzed using the Coding Potential Assessment Tool (CPAT), the CPAT raw score 0.0059 indicates a noncoding RNA (<0.364 coding probability cutoff). ³⁹

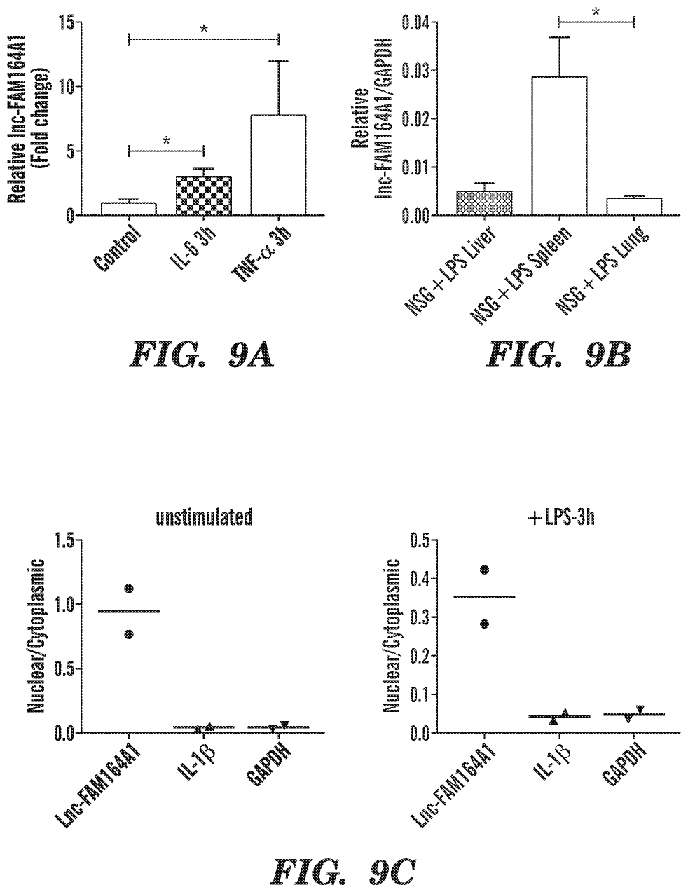

In human primary macrophages, LPS stimulation induced 67-fold and 41-fold increases in lnc-FAM164A1 expression at 3 and 6 hours, respectively (real-time PCR, FIG. 1B). Pro-inflammatory cytokines IL-6 and TNF-α also increased the expression of lnc-FAM164A1 expression in PBMC-derived macrophages (FIG. 9A). An approximate 7-fold increase in lnc-FAM164A1 expression was also detected in human PBMCs isolated from LPS-stimulated whole blood ex vivo (FIG. 1C). Furthermore, it was found that lnc-FAM164A1 expression increases in both lymphocytes and monocytes isolated from LPS-activated human whole blood ex vivo (data not shown). Lnc-FAM164A1 was conserved among human primates (such as Rhesus monkey) as analyzed using the UCSC Genome Browser (e.g., found on the world wide web at http://genome.ucsc.edu/index.html). Indeed, it was found that LPS induces a several-fold increase of lnc-FAM164A1 in macrophages differentiated from PBMCs isolated from whole blood of Rhesus monkey (data not shown).

Since lnc-FAM164A1 has no homolog in mice, a humanized NSG mouse model of endotoxemia was established to further seek in vivo significance. There months after the transplantation with human CD34+hematopoietic stem cells (HSCs), these humanized NSG mice contain 60-80% human CD45+leukocytes in their blood and spleens (data not shown). It was found that LPS challenge increased the expression of human lnc-FAM164A1 in white blood cells of humanized NSG mice (FIG. 1D). Higher levels of human lnc-FAM164A1 expression were found in the spleen than the lung of endotoxemic mice (FIG. 9B). Plasma of these mice contained abundant human TNF-α and IL-6 proteins (data not shown), indicating the transplanted human CD34+HSCs can differentiate into functional leukocytes in NSG mice.

LPS-induced Lnc-FAM164A1 locates in the cytoplasm and inside the nuclei.

The localization of lnc-FAM164A1 was then examined in macrophages. In situ hybridization using RNAscope (Advanced Cell Diagnostics) vidualized the expression of lnc-FAM164A1 RNA within macrophages. The low level of lnc-FAM164A1 located inside the nuclei of unstimulated macrophages, and LPS stimulation rapidly induced the de novo expression of lnc-FAM164A1 as early as 1 hour (data not shown) and peaking at 3 hours (FIG. 1E). Generally, LPS-induced Lnc-FAM164A1 located in the cytoplasm and inside the nuclei.

By separating nuclear RNA and cytoplasmic RNA and analyzing of lnc-FAM164A1 levels using RT-PCR, approximatelyl00% and 35% ratio of nuclear/cytoplasmic lnc-FAM164A1 was found in unstimulated and LPS-activated macrophages, respectively. In contrast, the nuclear/cytoplasmic ratio of protein coding genes (IL-1β and GAPDH) are less than 5% (FIG. 9C). Concomitant induction of lnc-FAM164A1 with pro-inflammatory cytokines in LPS-treated macrophages.

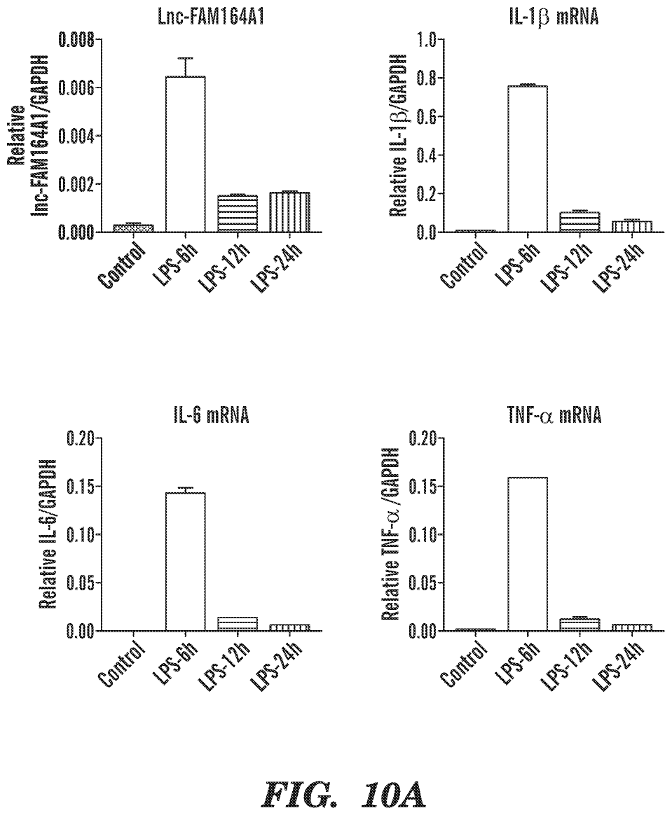

As mentioned, LPS triggers NF-κB signaling. Time-course experiments showed that lnc-FAM164A1 has higher expression at 6 hours, and waned 12 and 24 hours after the addition of LPS in human primary macrophages, which was similar to the kinetics of mRNA expression of the pro-inflammatory cytokines IL-1β, IL-6 and TNF-α, known targets of the NF-κB pathway (FIG. 10A). A specific inhibitor (Bay 11-7802) of NF-κB signaling blocks the transcription of lnc-FAM164A1 and cytokines in a dose-dependent manner (3-30 μM) (FIG. 10B). These data indicate that lnc-FAM164A1 is a target gene of NF-κB and may share similar or intertwined regulatory mechanisms with IL-1β, IL-6 and TNF-α in LPS-activated macrophages.

Silencing of lnc-FAM164A1 suppresses the expression of pro-inflammatory molecules in LPS-activated human macrophages.

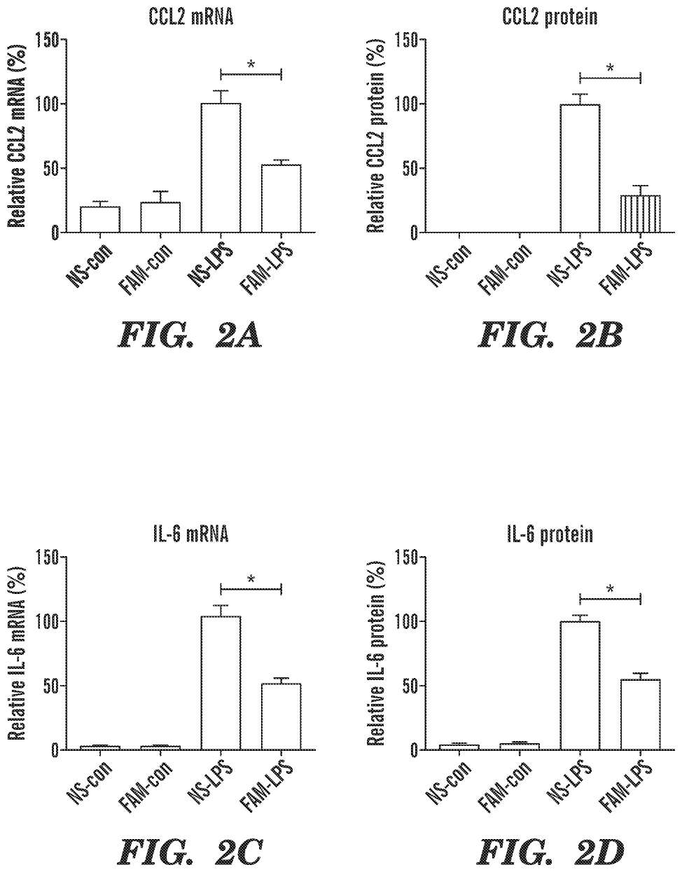

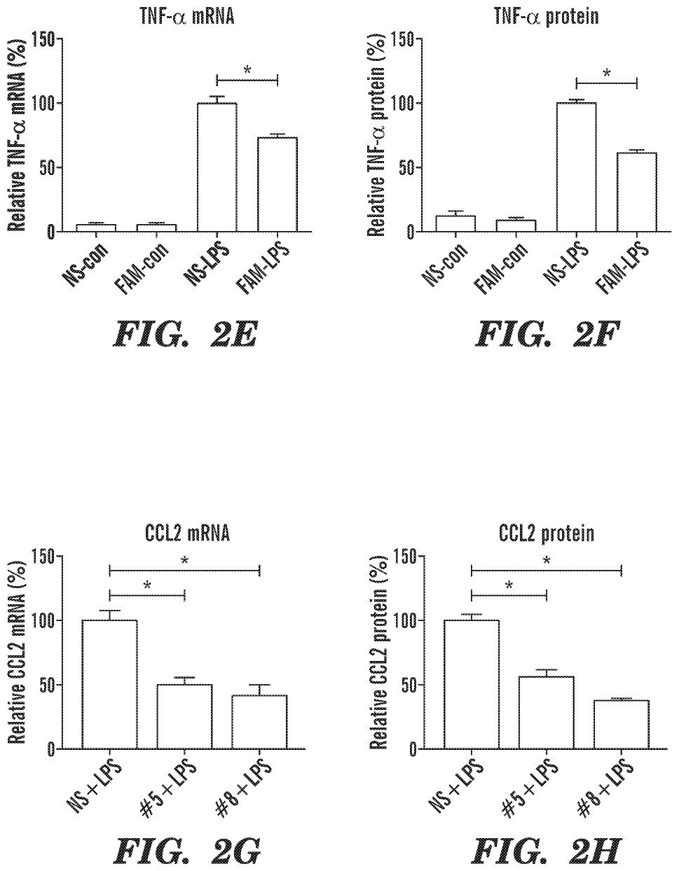

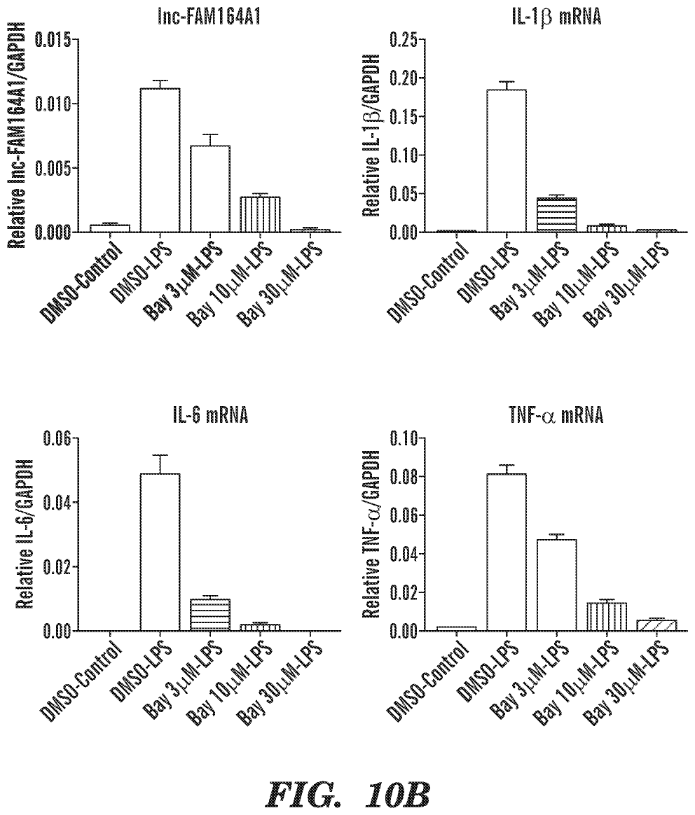

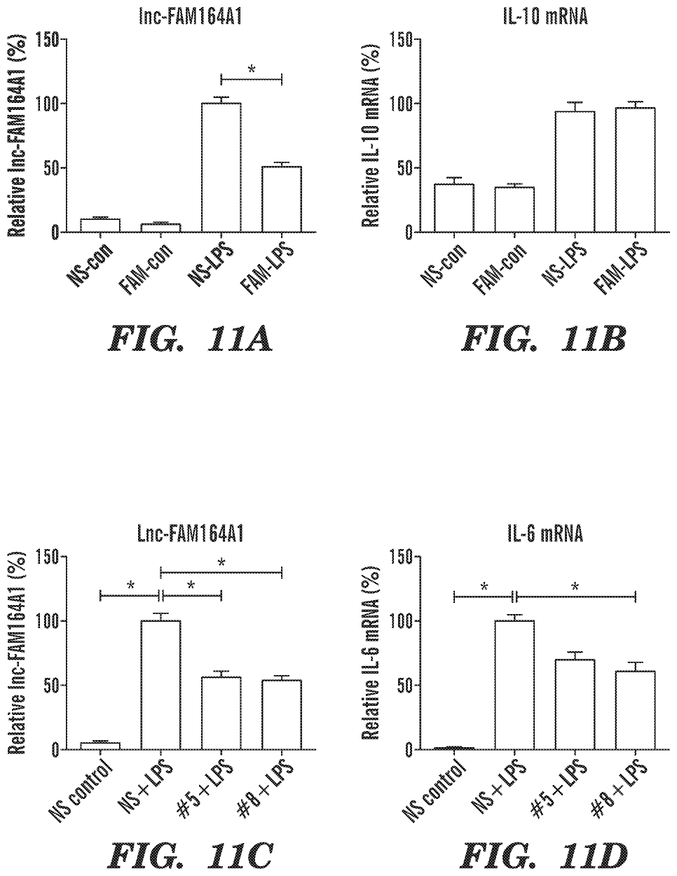

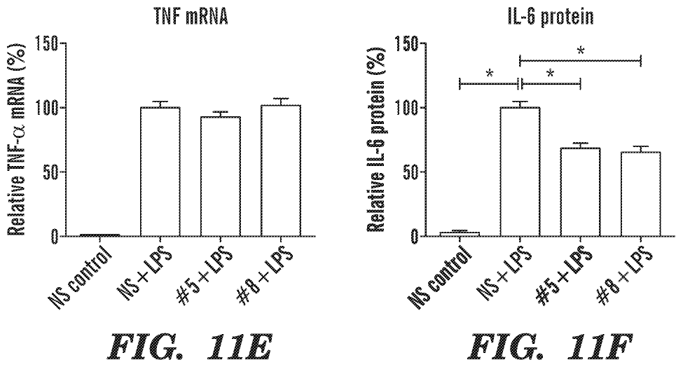

It was then determined whether lnc-FAM164A1 plays a causal role in pro-inflammatory activation of macrophages as gauged by the induction of cytokines and chemokines in LPS-activated human primary macrophages. To accomplished this, loss-of-function experiments were performed using anti-sense oligonucleotides (LNA™ long RNA GepmeR, Exiqon), which showed at least 50% silencing efficacy of lnc-FAM164A1 in human macrophages (FIG. 11A). While lnc-FAM164A1 silencing caused no changes in the expression of anti-inflammatory cytokine IL-10 in control and LPS-treated macrophages (FIG. 11B), the same treatment significant suppressed the LPS-induced mRNA expression and protein release of the pro-inflammatory chemokine CCL2 and cytokines IL-6 and TNF-α (FIG. 2A-2F). Inspired by cytoplasmic location of lnc-FAM164A1, 10 different siRNA oligonucleotides targeting lnc-FAM164A1 were also designed and their silencing efficacy was determined in human macrophages (Axolabs, Kulmback, Germany). Oligonucleotides #5 and #8 showed the higher potency of silencing than the others. siRNA silencing of lnc-FAM164A1 with these oligonucleotides also resulted in decreased expression of CCL2 (FIG. 2G, 211) and IL-6 (FIG. 11D, 11F) at mRNA and protein levels on LPS-stimulated human macrophages. No significant change of TNF-α mRNA was detected (FIG. 11E).

Enfored expression of lnc-FAM164A1 enhances LPS-induced TNF-α expression in macrophages.

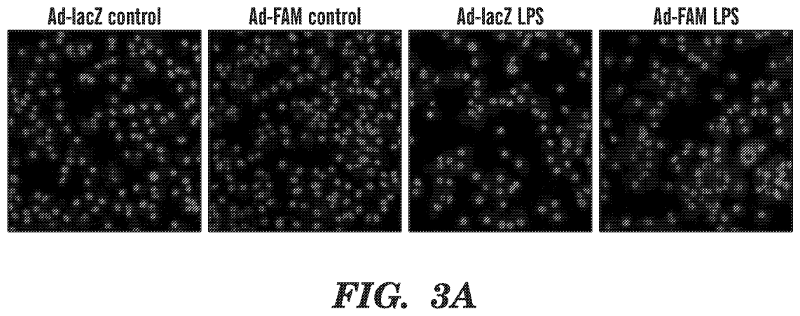

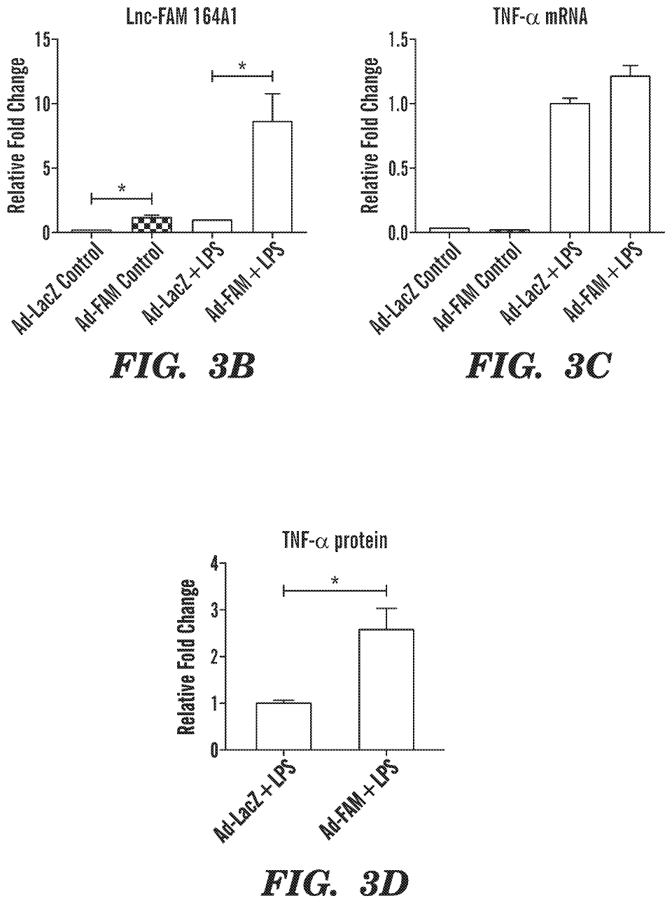

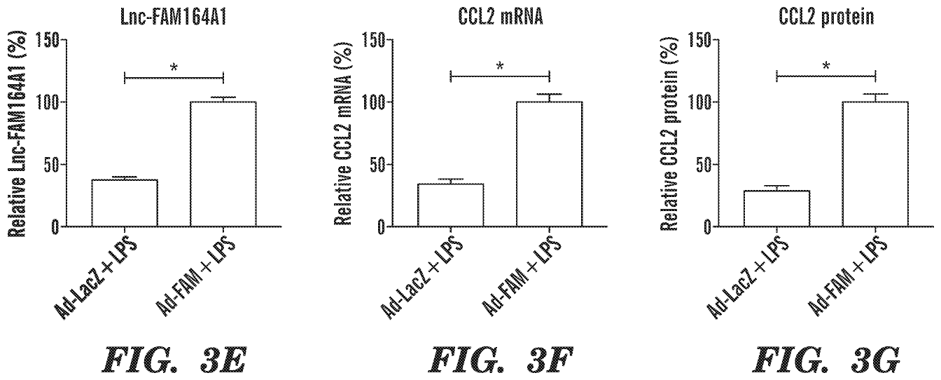

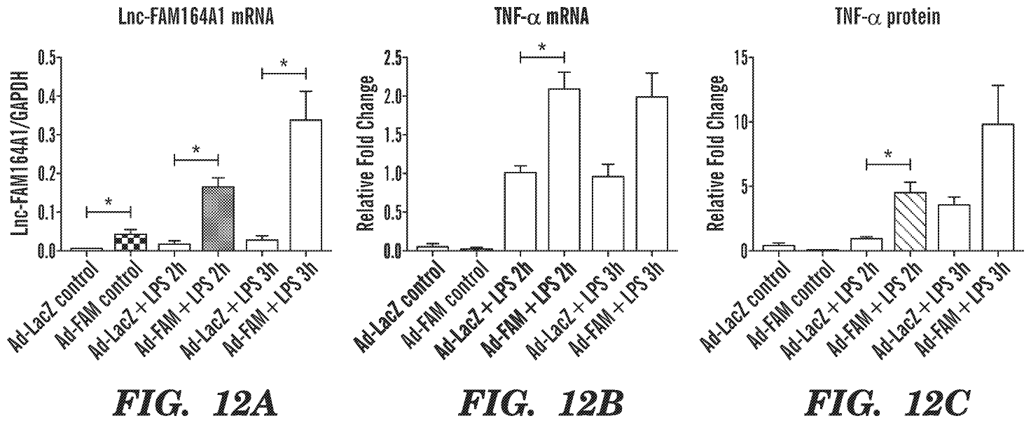

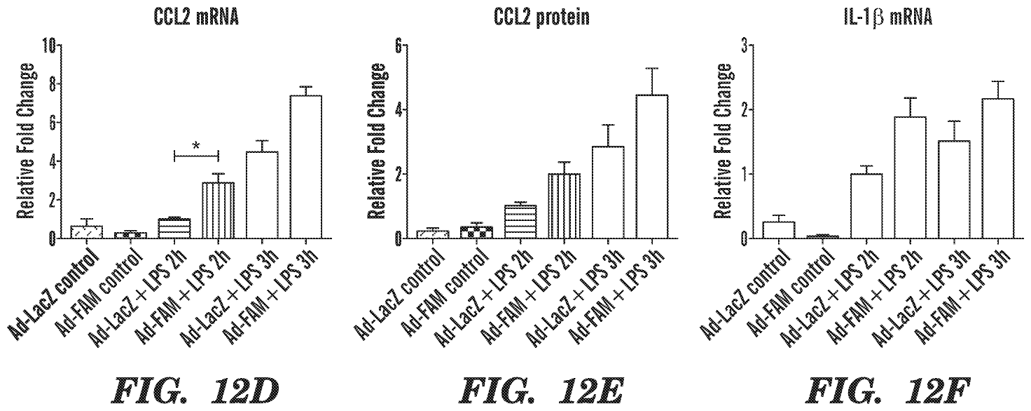

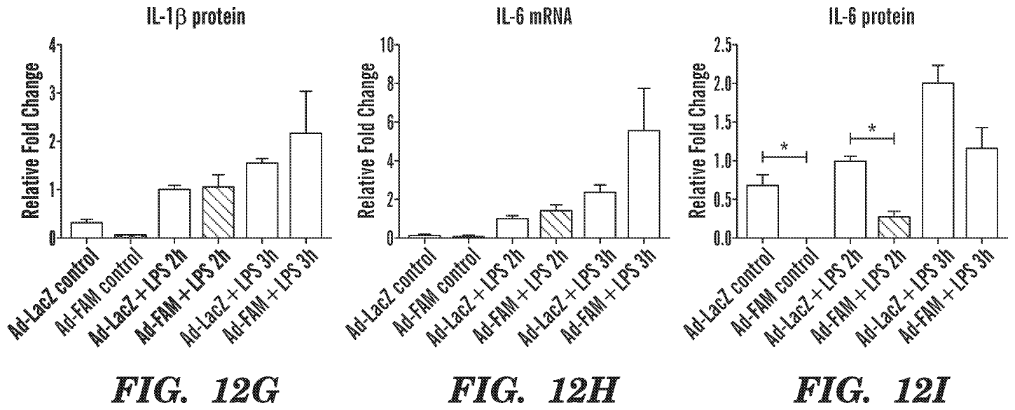

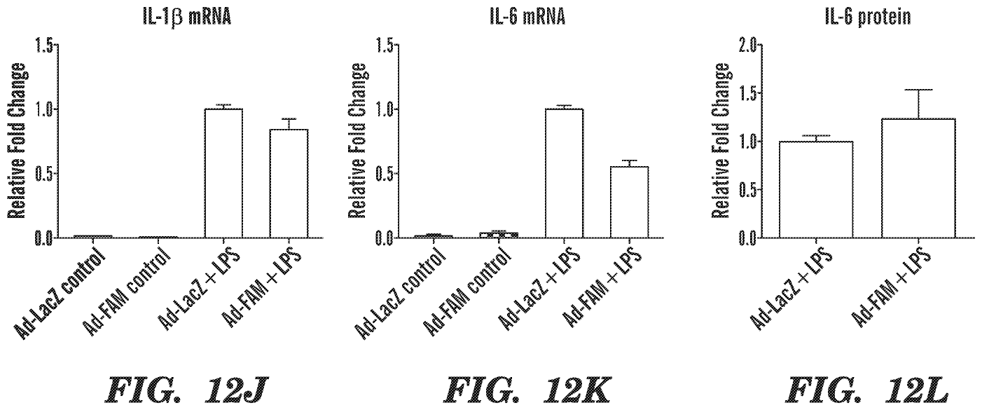

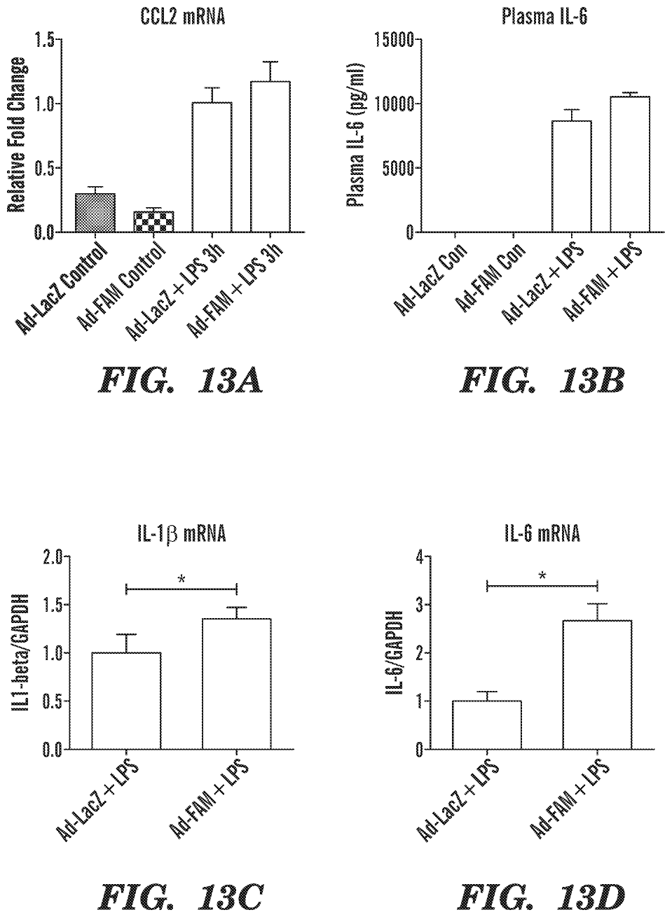

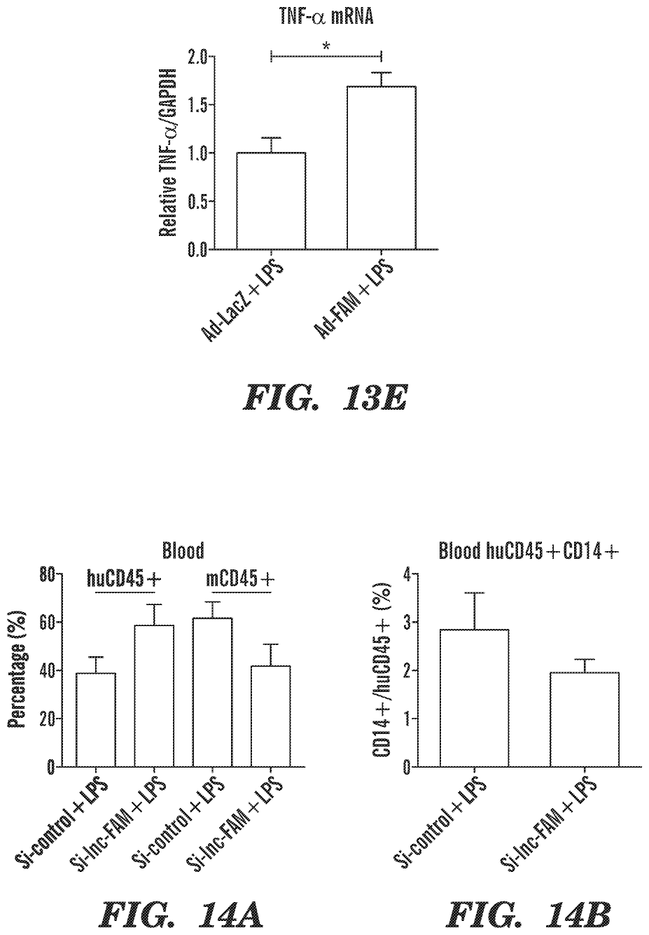

To further determine the causal role of lnc-FAM164A1 in pro-inflammatory activation of macrophages, gain-of-function experiments were performed using adenovirus expressing lnc-FAM164A1 (Ad-lnc-FAM164A1) or lacZ control in the human macrophage-like cell line THP-1 and human PBMC-derived macrophages.²² As compared with unstimulated THP-1 cells, Ad-lnc-FAM164A1 produced a 20-fold induction of lnc-FAM164A1 as compared with LacZ Control (FIG. 12A), which had no significant effects on the basal levels of TNF-α (FIG. 12B and 12C) and CCL2 (FIG. 12F and 121). LPS treatment enhanced lnc-FAM164A1 expression in THP-1 cells infected by Ad-lnc-FAM164A1 (FIG. 12A), which concomitantly associated with significant increases in CCL2 and TNF-α mRNA expression and TNF-α protein release in the culture media (FIG. 12B-12E). No significant changes in the expression of IL-1(3 was observed and decreased expression of IL-6 protein release in culture medium (FIG. 13F-131). Similarly, in human PBMC-derived macrophages, enforced expression of lnc-FAM164A1 enhanced LPS-triggered CCL2 expression (FIG. 3F and 3G) and TNF-α protein (FIG. 3D) release in the media from primary macrophages activated by LPS treatment (2 hour time-point), while no significant changes in TNF-α mRNA expression was found (FIG. 3C). No significant changes in the expression of IL-6 and IL-1β were observed (FIG. 12J-12L).

Lnc-FAM164A 1 triggers NF-κB signaling in LPS-activated macrophages.

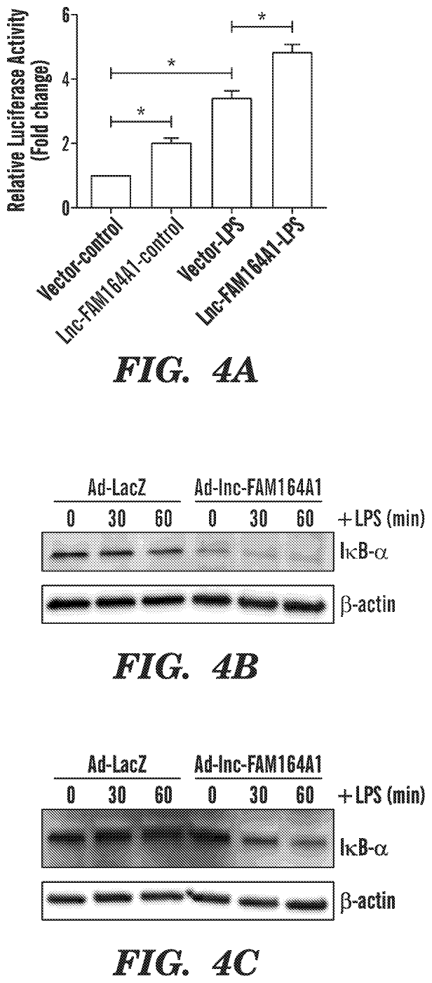

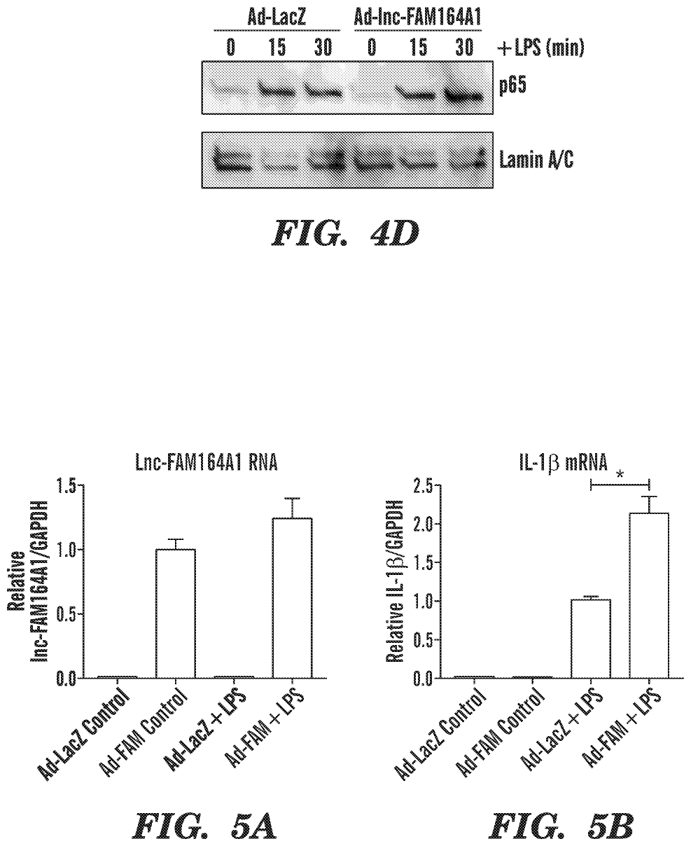

The NF-κB pathway plays a pivotal role in the transcriptional activation of pro-inflammatory factors such as IL-1β, IL-6 and TNF-α in LPS-elicited macrophages. Ligation of TLR4 with bacterial LPS triggers the signal transduction to induce the phosphorylation and the degradation of IκB proteins, followed by NF⊏B dimers translocating from cytoplasm to nucleus. Loss-of-function and gain-of-function data described herein indicate that lnc-FAM164A1 may promote TNF-α expression through its interaction with the NF-κB pathway. This was initially tested by using a luciferase reporter assay on HEK-293 cells. HEK-293 cells were co-transfected with either empty vector or lnc-FAM164A1 expression vector combined with pGL4.32[/uc2P/NF-κB-RE/Hygro] vector, which contains five copies of NF-κB response element (NF-κB-RE) driving the transcription of luciferase reporter gene (luc2P). Overexpression of lnc-FAM164A1 increased the luciferase activity in unstimulated or LPS-activated cells (FIG. 4A). Western blot analysis was then performed to detect the protein levels of/KB-a in whole-cell lysates and p65 transcription factor in the nuclear extracts. Expression of lnc-FAM164A1 by adenovirus infection induced rapid IKB-a degradation induced by LPS on both THP-1 cells (FIG. 4B) and human PBMC-derived macrophages (FIG. 4C). Further, enforced expression of lnc-FAM164A1 increased the p65 translocation and accumulation in the nucleus 30 mins after LPS stimulation in human PBMC-derived macrophages (FIG. 4D). These results indicate that lnc-FAM164A1 may regulate the expression of pro-inflammatory molecules through NF-κB signaling.

Enforced expression of lnc-FAM164A1 enhances TNF-α expression in a mouse model of endotoxemia in vivo.

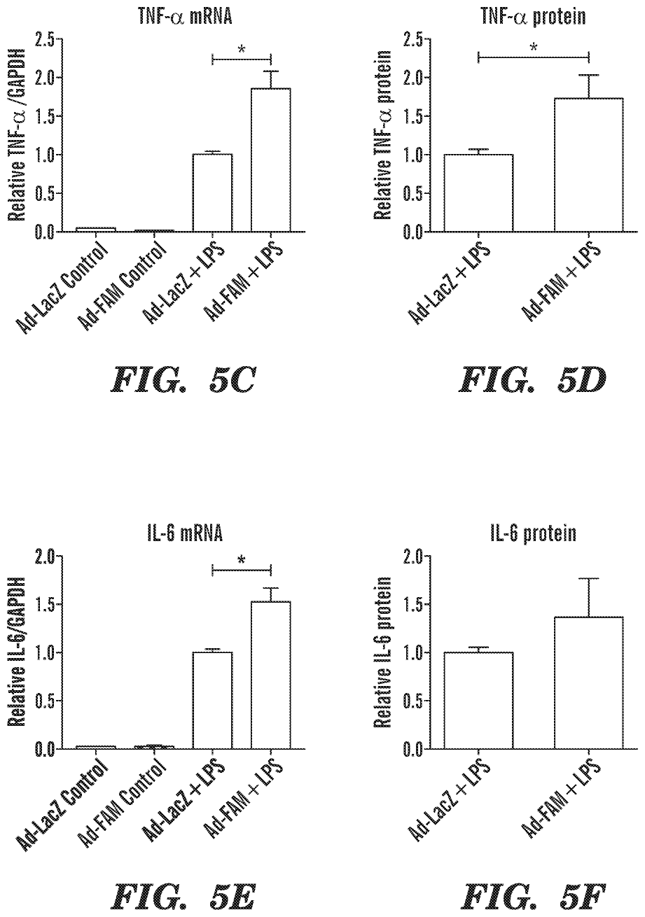

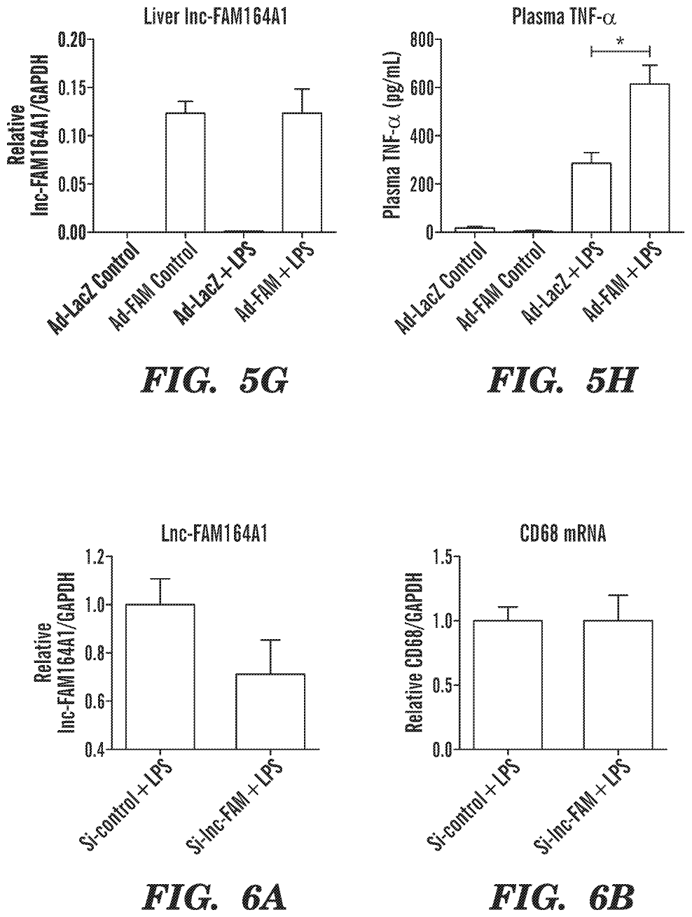

Although lnc-FAM164A1 has no homolog in mice, it is plausible the interacting protein(s) of lnc-FAM164A1 are conserved between human and mouse, which provides us the rational to perform gain-of-function studies in peritoneal macrophages isolated from wild-type C57BL6 mice. Ad-lnc-FAM164A1 induced high levels of lnc-FAM164A1 expression in mouse peritoneal macrophages (FIG. 5A). Consistent with the results found in human macrophages (FIG. 3), enforced expression of human lnc-FAM164A1 enhanced the expression of IL-10, IL-6 and TNF-α mRNAs (FIGS. 5B, 5C and 5E) and TNF-α protein (FIG. 5D) in LPS-elicited peritoneal macrophages without affecting the basal levels of these molecules in unstimulated cells. No changes of CCL2 mRNA (FIG. 13A) and IL-6 protein (FIG. 5F) were observed.

To determine whether lnc-FAM164A1 regulates pro-inflammatory gene expression in vivo, gain-of-function experiments were conducted using a well-established mouse model of endotoxemia. Three days after the administration of adenovirus, enforced expression of lnc-FAM164A1 were detected in the liver by RT-PCR (FIG. 5A). It was found that adenovirus infection alone had no effects on plasma levels of the pro-inflammatory cytokines TNF-α and IL-6 (ELISA, FIGS. 5H and 13B). However, a significantly enhanced level of TNF-α was detected in the plasma of endotoxemic mice infected with Ad-lnc-FAM164A1 after 3-hour stimulation with LPS (FIG. 5H). No significant change of plasma IL-6 protein was detected by ELISA (FIG. 13B). Endotoxemic mice infected with Ad-lnc-FAM164A1 have significantly higher mRNA levels of cytokines in the liver (FIG. 13C, 13D, 13E).

siRNA silencing of lnc-FAM164A1 in human monocyte/macrophages decreases human CCL2 and IL-6 expression in a humanized mouse model of endotoxemia.

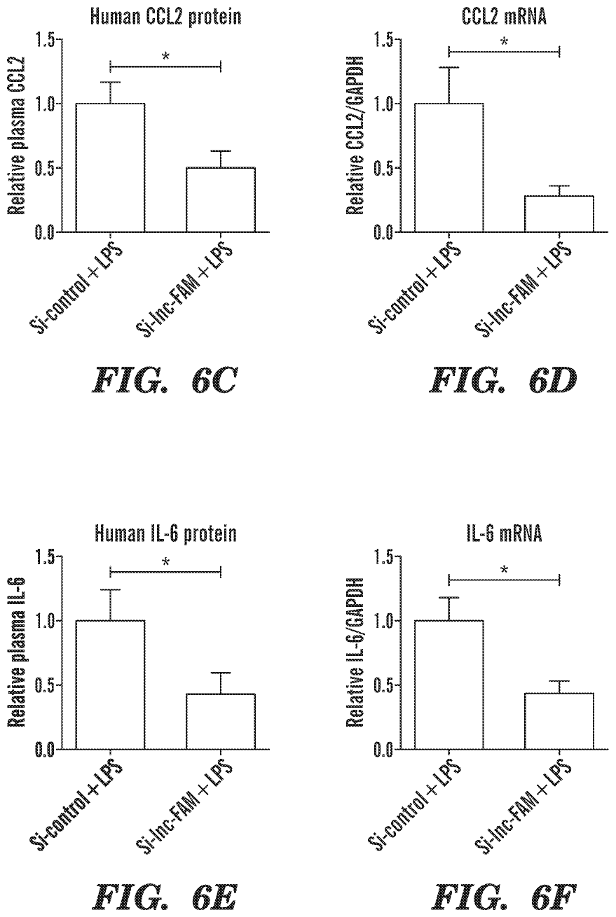

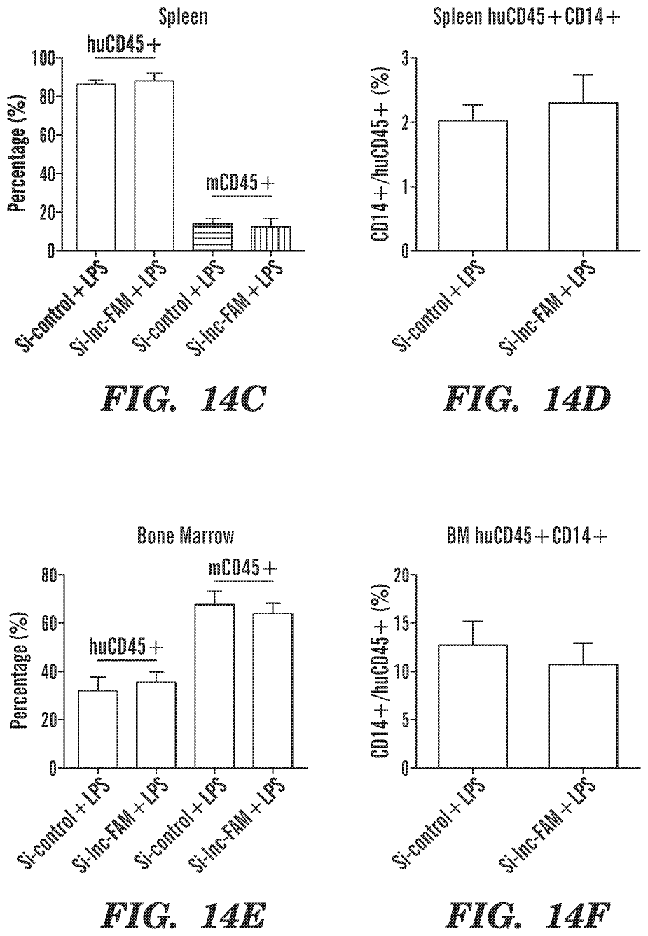

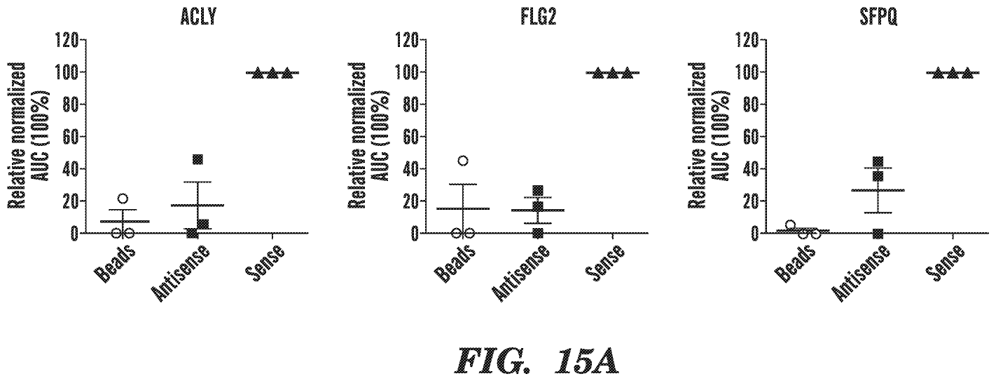

In order to determine the in vivo significance of lnc-FAM164A1 in human monocytes/macrophages, the loss-of-function study in recently developed humanized NSG-SGM3 mice was explored, which bear 30-80% human CD45+WBCs and 2-12% of monocytes in the blood, spleen and bone marrow after the transplantation with human CD34+HSCs (FIG. 14A-14F). lnc-FAM164A1 siRNA oligos or control siRNA oligos encapsulated in monocyte/macrophage-targeted lipid nanoparticles C12-200 were administrated to humanized NSG-SGM3 mice. A 30% decrease of lnc-FAM164A1 expression was found in purified human monocytes from lnc-FAM siRNA treated mice compared with control mice (FIG. 6A). Lnc-FAM164A1 siRNA-treated mice have significantly lower levels of human CCL2 and IL-6 protein in plasma (FIG. 6C and 6E), and decreased expression of human CCL2 and IL-6 mRNA in peritoneal cells (FIG. 6D and 6F). No change of human CD68 mRNA was detected (FIG. 6B) in peritoneal cells, which indicates a similar numbers of human macrophages accumulated in LPS-stimulated peritonitis.

RNA pulldown combined with Mass Spectrometry identified three proteins associated with lnc-FAM164A 1.

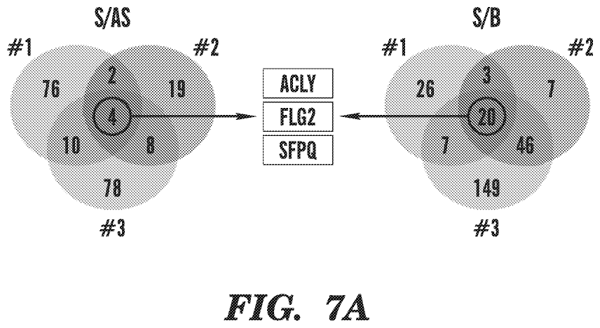

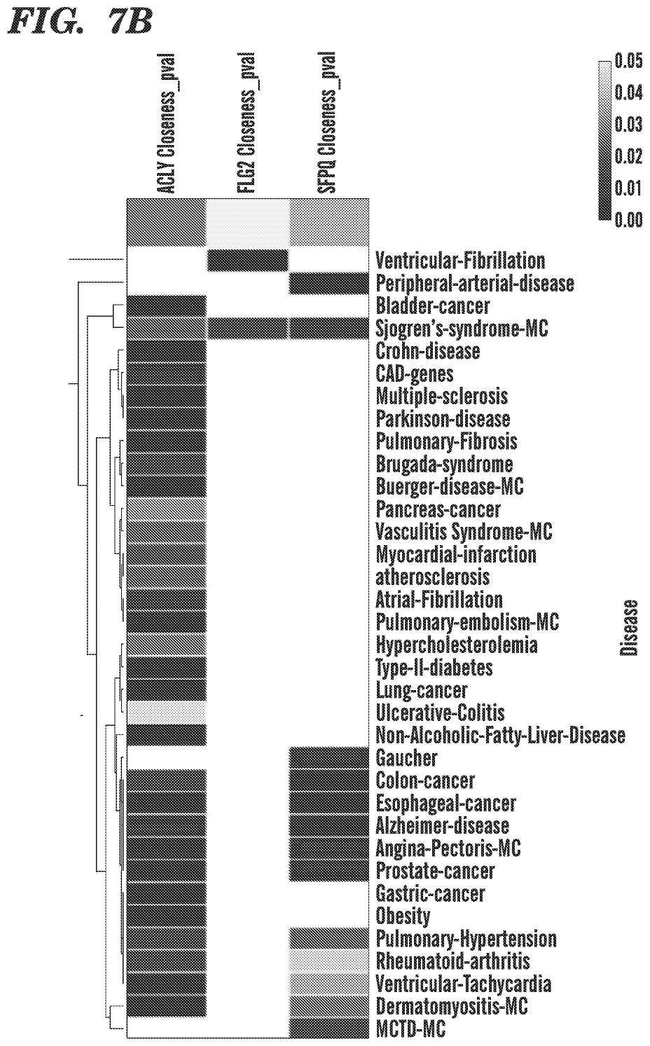

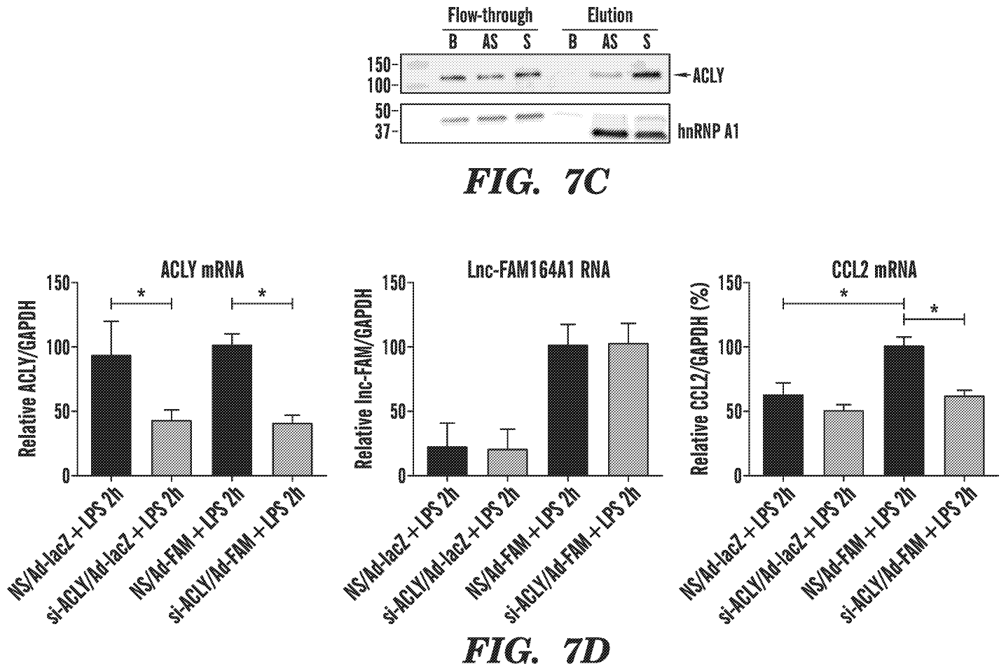

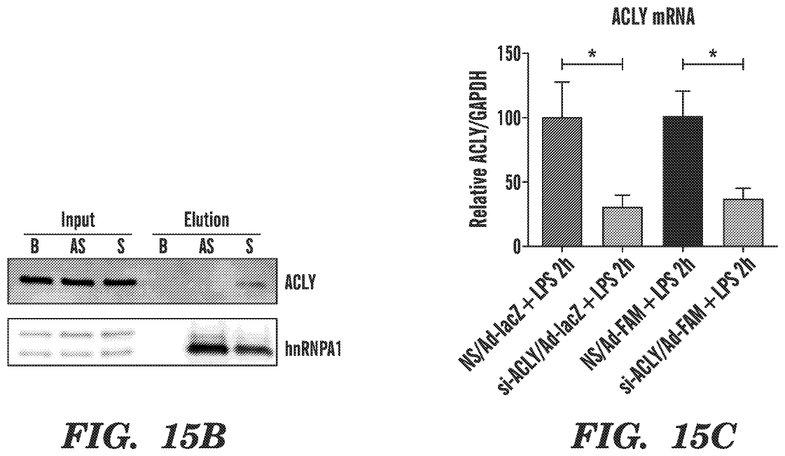

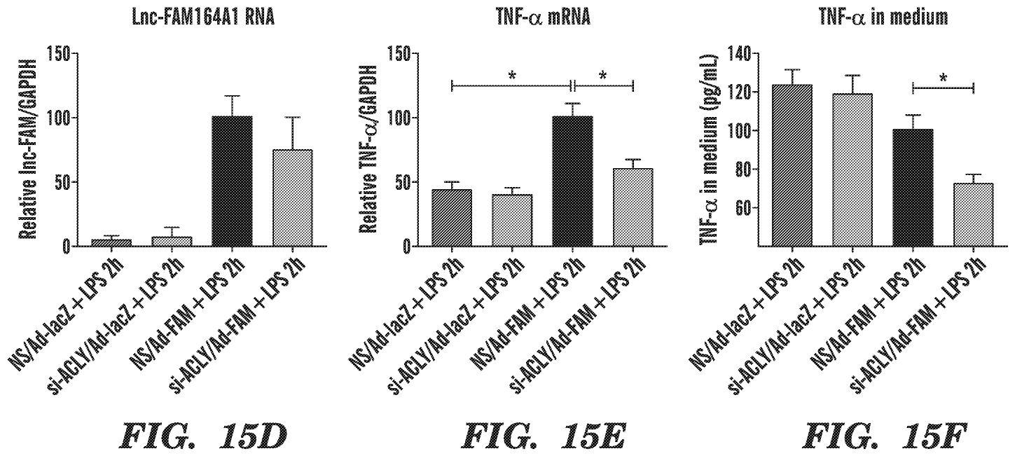

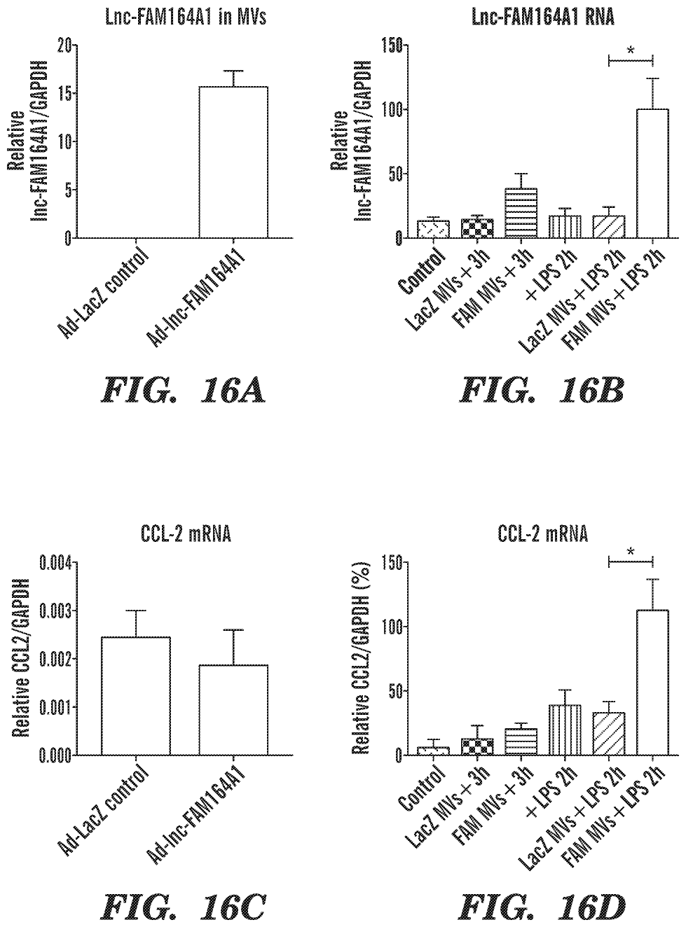

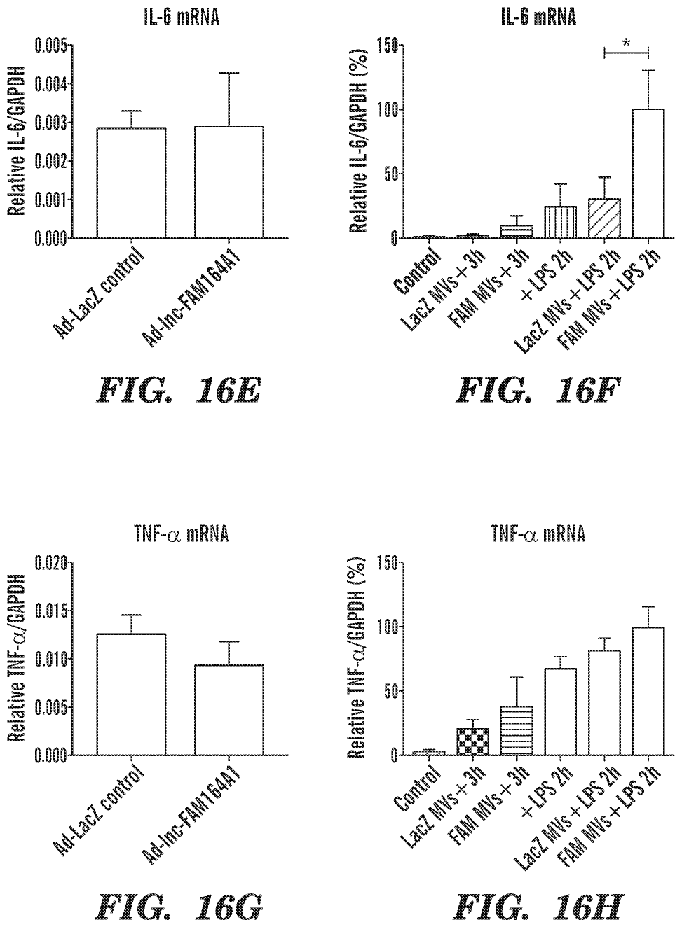

To address which proteins interact with lnc-FAM164A1 in LPS-activated THP-1-differentiated macrophages, lnc-FAM164A1 RNA pulldown assay was performed and analyzed the eluted proteins in three independent experiments by MS. Three unique proteins (ATP-citrate synthase: ACLY; Filaggrin-2: FLG2; Splicing factor, proline- and glutamine-rich: SFPQ) have more than 2-fold normalized AUC in the comparisons of lnc-FAM164A1 sense RNA versus antisense RNA (S/AS) and sense RNA versus beads controls (SB) (shown as Venn Diagram in FIG. 7A). Furthermore, these three proteins eluted from lnc-FAM164A1 sense RNA have significantly high normalized AUC compared with antisense RNA and beads controls (FIG. 15A, n=3, p<0.05 by t-test, Sense vs Beads; Sense vs Antisense).

Network analyss linked lnc-FAM164A 1 with human inflammatory diseases.

To computationally predict the role of lnc-FAM164A1 in human diseases, the disease associations of the interacting proteins ACLY, FLG2 and SFPQ through network analysis of the average shortest network distance between their modules and disease-related proteins were then examined. ACLY had significant relevance with metabolic and inflammatory disorders such as atherosclerosis, myocardial infarction, obesity, type 2 diabetes and pheumatoid arthritis (FIG. 7B).

Silencing of ACLY decreases the CCL2 and TNF-α expression induced by the enforced expression of lnc-FAM164A1 on LPS-activated cells.

RNA pulldown combined with western blot confirms the interaction of ACLY with lnc-FAM164A1 sense RNA in human PBMC-derived macrophages (FIG. 7C) and THP-1-differentiated macrophages (FIG. 15B). In contrast, heterogeneous nuclear ribonucleoprotein Al (hnRNPAl) was pulldown by both antisense and sense RNA of lnc-FAM164A1 (FIGS. 7C and 15B). Enforced expression of lnc-FAM164A1 increased ACLY protein expression with no change of ACLY mRNA levels in LPS-activated macrophage like cells THP-1 (data not shown). Finally, silencing of ACLY significantly decreases the CCL2 mRNA and TNF-α expression induced by the enforced expression of lnc-FAM164A1 on human primary macrophages (FIG. 7D) and THP-1 cells (FIG. 15D-15F), respectively.

Discussion

The present study aimed to identify the early response lnc-RNAs as potential key regulators of pro-inflammatory activation of human macrophages. Unbiased screening of LPS-elicited human primary macrophages using human lnc-RNA microarray identified 11 lnc-RNAs including lnc-FAM164A1. Limited information is available for lnc-FAM164A1. Two recent studies demonstrated the elevated expression of lnc-FAM164A1 in achilles tendons of old human donors and in THP-1 cells infected with Gram-negative bacteria Campylobacter concisus.^{40,41} lnc-RNA-lnc-RNA network analysis on ovarian cancer indicates a potential association of lnc-FAM164A1 with cell proliferation.' In bladder cancer, lnc-FAM164A1 (RP11-79H23.3) reguates cell proliferation, migration and tumorigenesis and lung metastasis in vivo.⁴³ However, the biological function of lnc-FAM164A1 in human macrophages remains unknown. Loss-of-function (silencing of lnc-FAM164A1 expression) and gain-of-function (adenovirus-induced enforced expression) experiments in the present study revealed lnc-FAM164A1 promotes inflammatory responses in LPS-activated human macrophages in vitro and mouse models of endotoxemia in vivo. ACLY was identified as one of the lnc-FAM164A1-associated proteins and lnc-FAM164A1/ACLY interaction contributes the expression of CCL2 and TNF-α induced by enforced expression of lnc-FAM164A1 in vitro.

It is shown herein that lnc-FAM164A1 is a target of NF-κB signaling and its expression levels increase with LPS, IL-6 or TNF-α stimulation in human primary macrophages. Inducible lnc-FAM164A1 in turn triggers NF-κB signaling cascades supported by several lines of evidence. The data provided herein thus indicate that lnc-FAM164A1 may enhance inflammation by accelerating a positive feedback loop of NF-κB activation. Mutiple lnc-RNAs (lincRNA-Tnfaips, lincRNA-Cox2 and MALAT1, FIRRE, Carlr) have been demonstrated playing roles in regulation of NF-κB signaling and the transcription of inflammatory genes in mouse and human macrophages.^{16,21,24,26,27} These findings further support the concept that NF-κB activity is precisely controlled at multiple levels by negative or positive regulatory elements.

Enforced expression of lnc-FAM164A1 increased the NF-κB activity in unstimulated HEK293 cells and induced the degradation of Ix13-a in unstimulated THP-1 cells and primary macrophages (FIG. 4). However, enforced expression of lnc-FAM164A1 with no LPS did not increase the expression of TNF-α at mRNA and protein levels on cultured macrophages in vitro and in plasma samples in vivo (FIG. 5). These set of data may indicate the crosstalk between lnc-FAM164A1 and NF-κB signaling to co-regulate the activation of NF-κB target genes in LPS-stimulated macrophages.

It is demonstrated herein that ACLY, as lnc-FAM164A1-associated protein, significantly contributes the expression of CCL2 and TNF-α induced by enforced expression of lnc-FAM164A1 in vitro (FIG. 7 and FIG. 15A-15F). It was also found that enforced expression of lnc-FAM164A1 increases the protein expression of ACLY without change of the ACLY mRNA in THP-1-differentiated macrophage cells (data not shown). In LPS-activated macrophages, the elevated expression of the citrate carrier (Slc25a1) leads to increased transport of citrate out of the mitochondria. Citrate can be metabolized by ACLY to generate oxaloacetate and acetyl-CoA. The latter provides the acetyl group for acetylation of histone proteins and transcription factors, including p50 and p65 subunits of NF-κB.⁴⁴ Therefore, enforced expression of lnc-FAM164A1 may increase cytosolic acetyl-CoA level and the acetylation of p65/p50, which promotes NF-κB activation ⁴⁵ and leads to the enhanced expression of proinflammatory cytokines in LPS-activated human macrophages.

Recent finding in bladder cancer has demonstrated lnc-FAM164A1 (RP11-79H23.3) shares the conserved target sites of miR-107 with PTEN, a negative regulator of PI3K/Akt signaling pathway'. Therefore, enforced expression of lnc-FAM164A1 in human macrophages can lead to increase PTEN expression and inactivation of PI3K/AKT signaling pathway, which negatively regulates NF-κB activation in LPS-activated monocytes/macrophages.^{46,47} Thus, lnc-FAM164A1 may also act as a sponge for miR-107 to regulate NF-κB signaling in human macrophages.

Previous studies reported that several human lnc-RNAs including lnc-RNA-THRIL, Lnc-RNA-TCONS_00019715 and lnc-MacORIS regulate cytokine expression and activation/polarization of macrophages. ^{22,23 29} Increasing list of lnc-RNAs emerge as novel regulators of inflammatory response in macrophages treated by a wide array of stimuli indicates that these distinct lnc-RNAs may play significant roles in the inflammatory and cardiometablic diseases. However, the majority of the human lnc-RNAs, including lnc-FAM164A1, has no homolog in mice, which brings challenges to determine the biological significance of lnc-RNAs in mouse models.

Herein, a humanized mouse model of endotoxemia was established to determine the in vivo significance of lnc-FAM164A1. Humanized NSG mice engrafted with CD34+HSCs or PBMCs have been widely used to study not only human biological processes in vivo but also human diseases including infectious diseases, autoimmunity, cancer and atherosclerosis. 48-51 _{In} a humanized NSG mouse model of endotoxemia, ⁵° LPS challenge increased the expression of lnc-FAM164A1 in blood leukocytes (FIG. 1D). The expression levels of lnc-FAM164A1 were higher in spleens than lungs (FIG. 9B) in endotoxemic NSG mice, which may be explained by the reservoir of human monocyte/macrophages in the spleen. ⁵² It was further demonstrated that si-RNA silencing of lnc-FAM164A1 by lipid nanoparticles specifically targeting monocyte/macrophages can result in a significant decrease of human CCL2 and IL-6 expression in plasma and peritoneal cells isolated from endotoxemic NSG-SGM3 mice (FIG. 6). Further analysis of human cytokine mRNA in different organs have found that silencing of lnc-FAM164A1 decreases the mRNA of proinflammatory cytokines in the lungs, but without change in liver and spleen (Data not shown), which may indicate alveolar macrophages significantly contribute to the systematic inflammation and septic lung injury. It was also found increased expression of lnc-FAM164A1 in PBMCs isolated from LPS-stimulated citrated human whole blood ex vivo (FIG. 1C). All these data indicated that the expression of lnc-FAM164A1 increases in the circulating PBMCs from septic patients. Lnc-FAM164A1 expression levels in blood maybe a useful biomarker for sepsis, which warrants further investigations.

### REFERENCES

1. Rinn J L, Chang H Y. Genome regulation by long noncoding RNAs. Annu Rev Biochem. 2012;81:145-166.
2. Ulitsky I, Shkumatava A, Jan C H, Sive H, Bartel D P. Conserved function of lincRNAs in vertebrate embryonic development despite rapid sequence evolution. Cell. 2011;147(7):1537-1550.
3. Liu S J, Horlbeck M A, Cho S W, et al. CRISPRi-based genome-scale identification of functional long noncoding RNA loci in human cells. Science. 2017;355(6320).
4. Quinn J J, Chang H Y. Unique features of long non-coding RNA biogenesis and function. Nat Rev Genet. 2016;17(1):47-62.
5. Batista P J, Chang H Y. Long noncoding RNAs: cellular address codes in development and disease. Cell. 2013;152(6):1298-1307.
6. Hu W, Yuan B, Flygare J, Lodish H F. Long noncoding RNA-mediated anti-apoptotic activity in murine erythroid terminal differentiation. Genes Dev. 2011;25(24):2573-2578.
7. Chen MT, Lin H S, Shen C, et al. PU.1-Regulated Long Noncoding RNA lnc-MC Controls Human Monocyte/Macrophage Differentiation through Interaction with MicroRNA 199a-5p. Mol Cell Biol. 2015;35(18):3212-3224.
8. Hu G, Tang Q, Sharma S, et al. Expression and regulation of intergenic long noncoding RNAs during T cell development and differentiation. Nat Immunol. 2013;14(11):1190-1198.
9. Wang P, Xue Y, Han Y, et al. The STAT3-binding long noncoding RNA lnc-DC controls human dendritic cell differentiation. Science. 2014;344(6181):310-313.
10. Witztum J L, Lichtman A H. The influence of innate and adaptive immune responses on atherosclerosis. Annu Rev Pathol. 2014;9:73-102.
11. Tabas I, Bornfeldt KE. Macrophage Phenotype and Function in Different Stages of Atherosclerosis. Circ Res. 2016;118(4):653-667.
12. Moore K J, Sheedy FJ, Fisher E A. Macrophages in atherosclerosis: a dynamic balance. Nat Rev Immunol. 2013;13(10):709-721.
13. Decano J L, Mattson P C, Aikawa M. Macrophages in Vascular Inflammation: Origins and Functions. Curr Atheroscler Rep. 2016;18(6):34.
14. Vaure C, Liu Y. A comparative review of toll-like receptor 4 expression and functionality in different animal species. Front Immunol. 2014;5:316.
15. Carpenter S, Aiello D, Atianand M K, et al. A long noncoding RNA mediates both activation and repression of immune response genes. Science. 2013;341(6147):789-792.
16. Hu G, Gong A Y, Wang Y, et al. LincRNA-Cox2 Promotes Late Inflammatory Gene Transcription in Macrophages through Modulating SWI/SNF-Mediated Chromatin Remodeling. J Immunol. 2016;196(6):2799-2808.
17. Tong Q, Gong A Y, Zhang X T, et al. LincRNA-Cox2 modulates TNF-alpha-induced transcription of Il 12b gene in intestinal epithelial cells through regulation of Mi-2/NuRD-mediated epigenetic histone modifications. FASEB J. 2016;30(3):1187-1197.
18. Reddy M A, Chen Z, Park J T, et al. Regulation of inflammatory phenotype in macrophages by a diabetes-induced long noncoding RNA. Diabetes. 2014;63(12):4249-4261.
19. Hu Y W, Zhao J Y, Li SF, et al. RP5-833A20.1/miR-382-5p/NFIA-dependent signal transduction pathway contributes to the regulation of cholesterol homeostasis and inflammatory reaction. Arterioscler Thromb Vasc Biol. 2015;35(1):87-101.
20. Atianand M K, Hu W, Satpathy A T, et al. A Long Noncoding RNA lincRNA-EPS Acts as a Transcriptional Brake to Restrain Inflammation. Cell. 2016;165(7):1672-1685.
21. Ma S, Ming Z, Gong A Y, et al. A long noncoding RNA, lincRNA-Tnfaip3, acts as a coregulator of NF-kappaB to modulate inflammatory gene transcription in mouse macrophages. FASEB J. 2017;31(3):1215-1225.
22. Li Z, Chao T C, Chang K Y, et al. The long noncoding RNA THRIL regulates TNFalpha expression through its interaction with hnRNPL. Proc Natl Acad Sci U S A. 2014;111(3):1002-1007.
23. Huang Z, Luo Q, Yao F, et al. Identification of Differentially Expressed Long Non-coding RNAs in Polarized Macrophages. Sci Rep. 2016;6:19705.
24. Zhao G, Su Z, Song D, Mao Y, Mao X. The long noncoding RNA MALAT1 regulates the lipopolysaccharide-induced inflammatory response through its interaction with NF-kappaB. FEBS Lett. 2016;590(17):2884-2895.
25. Das S, Reddy M A, Senapati P, et al. Diabetes Mellitus-Induced Long Noncoding RNA Dnm3os Regulates Macrophage Functions and Inflammation via Nuclear Mechanisms. Arterioscler Thromb Vasc Biol. 2018.
26. Lu Y, Liu X, Xie M, et al. The NF-kappaB-Responsive Long Noncoding RNA FIRRE Regulates Posttranscriptional Regulation of Inflammatory Gene Expression through Interacting with hnRNPU. J Immunol. 2017;199(10):3571-3582.
27. Castellanos-Rubio A, Kratchmarov R, Sebastian M, et al. Cytoplasmic Form of Carlr lnc-RNA Facilitates Inflammatory Gene Expression upon NF-kappaB Activation. J Immunol. 2017;199(2):581-588.
28. Sun D, Yu Z, Fang X, et al. Lnc-RNA GASS inhibits microglial M2 polarization and exacerbates demyelination. EMBO Rep. 2017;18(10):1801-1816.
29. Zhang H, Xue C, Wang Y, et al. Deep RNA Sequencing Uncovers a Repertoire of Human Macrophage Long Intergenic Noncoding RNAs Modulated by Macrophage Activation and Associated With Cardiometabolic Diseases. J Am Heart Assoc. 2017;6(11).
30. Fung E, Tang S M, Canner J P, et al. Delta-like 4 induces notch signaling in macrophages: implications for inflammation. Circulation. 2007;115(23):2948-2956.
31. Koga J, Matoba T, Egashira K, et al. Soluble Flt-1 gene transfer ameliorates neointima formation after wire injury in flt-1 tyrosine kinase-deficient mice. Arterioscler Thromb Vasc Biol. 2009;29(4):458-464.
32. Iwata H, Goettsch C, Sharma A, et al. PARP9 and PARP14 cross-regulate macrophage activation via STAT1 ADP-ribosylation. Nat Commun. 2016;7:12849.
33. Goettsch C, Hutcheson J D, Aikawa M, et al. Sortilin mediates vascular calcification via its recruitment into extracellular vesicles. J Clin Invest. 2016;126(4):1323-1336.
34. Menche J, Sharma A, Kitsak M, et al. Disease networks. Uncovering disease-disease relationships through the incomplete interactome. Science. 2015;347(6224):1257601.
35. Liu C C, Tseng Y T, Li W, et al. DiseaseConnect: a comprehensive web server for mechanism-based disease-disease connections. Nucleic Acids Res. 2014;42(Web Server issue):W137-146.
36. Rappaport N, Nativ N, Stelzer G, et al. MalaCards: an integrated compendium for diseases and their annotation. Database (Oxford). 2013;2013:bat018.
37. Wang J G, Manly D, Kirchhofer D, Pawlinski R, Mackman N. Levels of microparticle tissue factor activity correlate with coagulation activation in endotoxemic mice. J Thromb Haemost. 2009;7(7):1092-1098.
38. Ritchie M E, Phipson B, Wu D, et al. limma powers differential expression analyses for RNA-sequencing and microarray studies. Nucleic Acids Res. 2015;43(7):e47.
39. Volders P J, Helsens K, Wang X, et al. LNCipedia: a database for annotated human lnc-RNA transcript sequences and structures. Nucleic Acids Res. 2013;41(Database issue):D246-251.
40. Peffers M J, Fang Y, Cheung K, Wei T K, Clegg P D, Birch H L. Transcriptome analysis of ageing in uninjured human Achilles tendon. Arthritis Res Ther. 2015;17:33.
41. Kaakoush N O, Deshpande N P, Man S M, et al. Transcriptomic and proteomic analyses reveal key innate immune signatures in the host response to the gastrointestinal pathogen Campylobacter concisus. Infect Immun. 2015;83(2):832-845.
42. Li Y, Chen J, Zhang J, et al. Construction and analysis of lnc-RNA-lnc-RNA synergistic networks to reveal clinically relevant lnc-RNAs in cancer. Oncotarget. 2015;6(28):25003-25016.
43. Chi H, Yang R, Zheng X, Zhang L, Jiang R, Chen J. Lnc-RNA RP11-79H23.3 Functions as a Competing Endogenous RNA to Regulate PTEN Expression through Sponging hsa-miR-107 in the Development of Bladder Cancer. Int J Mol Sci. 2018;19(9).
44. Kelly B, O'Neill L A. Metabolic reprogramming in macrophages and dendritic cells in innate immunity. Cell Res. 2015;25(7):771-784.
45. Chen L, Fischle W, Verdin E, Greene W C. Duration of nuclear NF-kappaB action regulated by reversible acetylation. Science. 2001;293(5535):1653-1657.
46. Luyendyk J P, Schabbauer G A, Tencati M, Holscher T, Pawlinski R, Mackman N. Genetic analysis of the role of the PI3K-Akt pathway in lipopolysaccharide-induced cytokine and tissue factor gene expression in monocytes/macrophages. J Immunol. 2008;180(6):4218-4226.
47. Schabbauer G, Tencati M, Pedersen B, Pawlinski R, Mackman N. PI3K-Akt pathway suppresses coagulation and inflammation in endotoxemic mice. Arterioscler Thromb Vasc Biol. 2004;24(10):1963-1969.
48. Shultz L D, Ishikawa F, Greiner D L. Humanized mice in translational biomedical research. Nat Rev Immunol. 2007;7(2):118-130.
49. Moser J, van Ark J, van Dijk M C, et al. Distinct Differences on Neointima Formation in Immunodeficient and Humanized Mice after Carotid or Femoral Arterial Injury. Sci Rep. 2016;6:35387.
50. Skirecki T, Kawiak J, Machaj E, et al. Early severe impairment of hematopoietic stem and progenitor cells from the bone marrow caused by CLP sepsis and endotoxemia in a humanized mice model. Stem Cell Res Ther. 2015;6:142.
51. Dai C, Kayton N S, Shostak A, et al. Stress-impaired transcription factor expression and insulin secretion in transplanted human islets. J Clin Invest. 2016;126(5):1857-1870.
52. Swirski F K, Nahrendorf M, Etzrodt M, et al. Identification of splenic reservoir monocytes and their deployment to inflammatory sites. Science. 2009;325(5940):612-616.

### Example 2

Methods for Generating Extracellular Vesicles

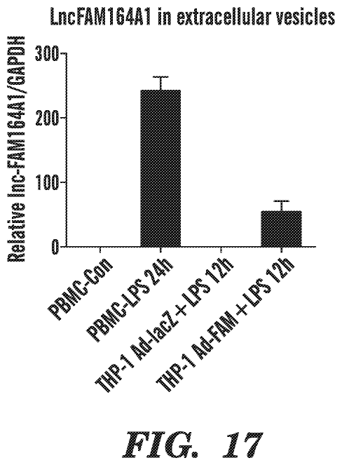

Culture media were collected from either Adenovirus infected RAW264.7 cells for 24 hours or LPS-activated human PBMC-derived macrophages or THP-1-differentiated macrophage cells. After two-time centrifugation at 1500g for 15 min to remove cells and cellular debris, the extracellular vesicles (EVs) was pelleted by ultracentrifugation at 100,000g for 1 hour and resuspended in normal growth medium at a 10-fold enrichment. RAW cells-derived EVs were added into RAW264.7 cells and incubated for one hour, followed by addition of 10 ng/mL of LPS for 2 hours. Total RNA was extracted from either EVs or RAW cells and used for cDNA synthesis. Expression of lnc-FAM164A1 RNA and mRNAs of mouse cytokine was determined by RT-PCR and normalized by GAPDH. FIG. 17 presents data showing lnc-FAM164A1 in extracellular vesicles.

### Example 3

Described herein are sequences for lnc-RNA found to be upregulated by LPS. The lnc-RNA include, but are not limited to: AL078621.3; RP11-536K7.5; RP11-143M1.3; RP11-561O23.5; AC10980.2; P2RX7; LOC440896; D63785; RP11-58A12.2; RP11-79H23.3 (lnc-FAM164A1); and RP13-452N2.1. SEQ ID NO: 1 is the the nucleotide sequence of lnc-FAM164A1.

| SEQ ID NO: 28 is the nucleotide sequence of D63785. | |
|---|---|
| (SEQ ID NO: 28) | |
| tgcccttgctgtcctcctctgcaccatggctctctgcaaccagttctctg | |
| catcacttgctgctgacacgccgaccgcctgctgcttcagctacacctcc | |
| cggcagattccacagaatttcatggctgactactttgagacgagcagcca | |
| gtgctccaagcccagtgtcatcttcctaaccaagagaggccggcaggtct | |
| gtgctgaccccagtgaggagtgggtccagaaatacgtcagt | |
| SEQ ID NO: 39 is the nucleotide sequence of RP11-56023.5. | |
| (SEQ ID NO: 39) | |
| CTGAGTGCCACGTGCTGACGTCACTAAGATCAATACAGCAAACTCTGAAAGATGGACAGAGAGACAGGAGATGG | |
| TCCTTTATAATGCAGTGTGATCTGTGCTGCAATAGAGTTTTGGAAGCTAGAAGTTCAAAACGAGGTGTTGGCAG | |
| CACCATGCTCTCTCTGAAGATGCTAGGAAGAATCTGCTCCATGCCTTTCCATTCGCTCCTGGGGTTTCCTGCAA | |
| GCCCTGACATTCCTTGGCTTGTAGATGCATCACCCCAGTTTCCGCCCCCATCATCACATGGCCTCCTCTCTGTG | |
| TGTGCCTCTGCGTTCCCTCTATTCTTCTTCTAAGGACACCGACACCAGTCATAGTGGATTAAGGGTCCACTCCT | |
| AACCAATTACATCTGCAACAACCCTATTTCCAAATAAAGTCACATTCTAAGATTCCTAGGGAGAACCTGAATTT | |
| TTGGGGGTGTGTGGATACTGTTCAACCTGGGATATGGAGTAAATAAATGGCAAGGAAGATTACAGATGACTTTA | |
| AGCTAAAGAGGGAGATAGGGTTAAATGTAGGACTTTTTTTTTTTCCAGGACGGGAGAGGTTTAAACATGTTACT | |
| ACATTGAATAAATGAAATAATCACG | |
| SEQ ID NO: 30 is the nucleotide sequence of RP11-143M1.3. | |
| (SEQ ID NO: 30) | |
| CTGAGTGCCACGTGCTGACATCACTAAGATCAATACAGCAAACTCTGAAAGATGGACGGAGAGACAGGAGATGG | |
| TCCTTTATAATGCAGTGTGATCTGTGCTGCAATAGAGTTTTGGAAGCTAGAAGTTCAAAACGAGGTGTTGGCAG | |
| CACCATGCTCTCTCTGAAGATGCTAGGAAGAATCTGCTCCATGCCTTTCCATTCGCTTCTGGGGTTTCCTGCAA | |
| GCCCTGACATTCCTTGGCTTGTAGATACACCACCCCAGTTTCCGCCCCCATCATCACATGGCCTCCTCTCTGTG | |
| TGTGCCTCTGCGTTCCCTCTATTCTTCTTCTAAGGACACCGACACCAGTCATAGTGGATTAAGGGTCCACTCCT | |
| AACCAATTACATCTGCAACAACCCTATTTCCAAATAAAGTCACATTCTAAGATTCCTAGGGAGAACAGGAATTT | |
| TTGGGGGTGTGTGGATACTGTTCAACCTGGGACATGGAGTAAATAAATGGCAAGGAAGATTACAGATGACTTTA | |
| AGCTAAAGAGGGAGATAGGGTTAAATGTAGGACTTTTTTTTTTTCCAGGACGGGAGAGGTTTAAACATGTTACT | |
| ACATTGAATAAATGAAATAA | |
| SEQ ID NO: 31 is the nucleotide sequence of LOC440896. | |
| (SEQ ID NO: 31) | |
| AAAGTTTGGGATTCCCAGTCTACAAAACCCCACAGCTCCTAGGAATTCTCTCACCACCCTTGTGCCTTTAGGCT | |
| TCGGTAGATTGCAAATGACCTGCTTTCTTTCGGATCCCGGGCTGCTTTCGGACACCTGTCGAATAGTAAATCCC | |
| AAGTAAGGTACCTGCGGTCGTCGGCAGATCTGAATTTTCTTCTTGGACACCTAATACCCACAGTCCTCCAGAGA | |
| CCGAGAGGTTGATGTCACTCCCAATATCGGAGGAAGTATACACCCCCGTGTGAGATGGTCCTTAATAATATTCC | |
| ACGGCGGAGGGGGTGATATGACTACATACATGGCAGAAAGTGGAAACCTCCCAGGGATATTGTTCCCACGATCC | |
| TGGAGGGAAGAAGATGATGTTACTTTCAATATGACAGAAGGTGGATGAAGTGGTGGACTGCCCCTCCACACCTG | |
| TGGACACGCCCCCACTGATATTCCTTCTAATTGCAGCGTGGGAGAGGAGGATATGACACGCGATATCGCAGGGA | |
| GTAGAAACACCCCTGTGATACTGTTCTTAATATTCAGGGAGGAAGAGGATGATATTACTCCCAATACAGACGGG | |
| TGTACACCCTCTGTACACCGAGGGTGTACACCTGTCTGTGAAAGAGTTCGTAATCTCCAGAGGGGGAGATGATA | |
| TTACTCACAATATGGTAAAGAGGCTGTGAGTCCACGGAGGATCCTCAGAGCCAGCGGGGGAAGAGGGGCTGGCT | |
| CTCAGTCCCCGCCTCGCGGGGGTGACTCCCCCCAGTGCGATGGGGGTCCTAAGAGCCAGTGGGGGAAGAGGGGC | |
| TGGCTCTGAGACCCCGCCTCGCGGGGGGTGCCTCCCCGCCCTGTGATGGGGGTCCGAAGAGCCAGAAGGCTTAG | |
| AGGGGCTGGCTCTCAGTCCCCGCCTCGCGGGGGGTGCCTCCTCCCCCTGCGATGGGGGTCGTAAGAGCCACTGG | |
| GGGAATAGGGGCTTGCTCTCAGTCACCGCCTCGCGGGGGGTGCCTCCTTTCCTTTCTACATAGACACAGTGACA | |
| GTCTGATCTCTCTTTCTTTTCCCTACAGATGGACACGCCCCCACTGATATTGTTTTTAATGCAGCGTGGGAGGG | |
| GAGGATATGACACGCGATATCGCAGGTAGTAGAAACACCCCTTTGATAGTGTTCTTAATATTCAGGGAGGAAGA | |
| AGATGATGTTACTCCCAATACAGACGGATGTACACCCTCTGTACACCGAGGGTGTACACCCGTCTGTGAAGGAG | |
| TTCGTAATCTCCAGAGGTGGAGATGATATTACTCACAATATGGTAAACAGGCTGTGAGTCCATCGCGGATCCTC | |
| AGAGCCAGGTGGGGAAGAGGGGCTGGCTGTCAGTCCCCTCCTCGCGGGGGGTGCCTCCCCCACTGCTATGGGGA | |
| TCCCAAGAGCCAGTGGGGGAAGAGGTGCTGGCTCTCAGTCTCCGCCTCGCGAGGTGCCTCCCCACCCTGCGATC | |
| GGGGTCCGAAGAGCCAGGGGGGAAGAGGGGCTGGCTCTCTTCGTGGATGATTCTTTTTCCATTCTCAGGCAGTT | |
| TTCTTTTTTCTTTCTTTCTTTTTTTTTTTTTTTGAGACTGAGTCTTGCTCTGTTGCCCATGCTTTGCTCGATCT | |
| CGGGTGACTGCAACCACTGCCTCCCAGGTTCAAGAGATTCTCCTGCCTCAGCCTCCTGAGTAGCTGGGACTAGA | |
| GGCGTGTGTCACCACACCCAGCTAATTTTTGTATTTTTAGTAGAGATGGGGTTTCACCATGTTTGCCAGGATGG | |
| TCTCTATCTCCTGCCCTTGTGATCCACCCACCTCAGCCTCCCAAAGTGCTGGGATTGCAGGTGCGAGCCACCGG | |
| GTCCAGCCTCTCAGGCGATTTTCATACCTGCATACTCTGGTCACTACTCTGTTAAACAGTCAAGGAGGGTAAGT | |
| ATTATCTTCAGATTTCCAGAGCTCTGTCTCTGTACAGCCCTCTCCTCCTCAATATTCTGCCCTATGAATTCTAG | |
| CCACATTGGCCTTCCCAGGCTCACAGTTCTGTCTTCTCAACTCAGGAAGATCTCTGAGTTCCATCTGCATTCTT | |
| TCTTCCTGTGCTGTGGCCTGGAAAGTTTTCTAAGGTGTTAGGGAGGTCAATTGTGGGGCTAGCCTCATTTGTTT | |
| CTCATCTCTTGAGGATCACTGCCCTTTGATGCTTGATTCCAGTGATTGATTCCCTTTGTTGCTTGAGGGCCATA | |
| GTTTCATATATTTTGTCCAGTAGTTTTGTTGTTTTAGGTCAGAAAGTAATTTTGGTCTCTGTTACTCTATCTTG | |
| GCCAGAAGTGTAAGACCTAAGCATTTACACATCAAAATACTGCACACATAATTTTAGTTTAAGCTACTTTTTAA | |
| AAAATCTCCTTCATTTTCCATTTAGCATTCTATTTAGGGTATTACATTGGTTTTTTTGAAATTCTGTTATTGGC | |
| AGTTTCTATTGCCTATCAATCCCATTTAAAGATAGTGCATAGGGTATTCTAAAATAGCTGTTAAGCAAAGAGAA | |
| AATTGGGCCTGATAGGGTGAGAATCACAGCTCTAATACCTAGAGTGACCTTATAATGTATTGTCCAAAGGAGAT | |
| ATTTTTGACAGTGAAAGAGGGTGTTGTTAGTAATTATATCAGGACCATGGCCTAAACCAGGACTATCCCAGGAA | |
| GCCTGGGACATATTTGTACCCCATCTCTATTTAATGCCTTTATACAATTCTTTACTTAATTCTACCAGCCTTTA | |
| TTGAGCCTGCTTTCTTTGTCTAGCTGAGTGCCACGTGCTGACGTCACTAAGATCAATACAGCAAACTCTGAAAG | |
| ATGGACAGAGAGACAGGAGATGGTCCTTTATAATGCAGTGTGATCTGTGCTGCAATAGAGTTTTGGAAGCTAGA | |
| AGTTCAAAACGAGGTGTTGGCAGCACCATGCTCTCTCTGAAGATGCTAGGAAGAATCTGCTCCATGCCTTTCCA | |
| TTCGCTTCTGGGGTTTCCTGCAAGCCCTGACATTCCTTGGCTTGTAGATGCATCACCCCAGTTTCCGCCCCCAT | |
| CATCACATGGCCTCCTCTCTGTGTGTGCCTCTGCGTTCCCTCTATTCTTCTTCTAAGGACACCGACACCAGTCA | |
| TAGTGGATTAAGTGTCCACTCCTAACCAATTACATCTGCAACAACCCTATTTCCAAATAAAGTCACATTCTAAG | |
| SEQ ID NO: 32 is the nucleotide sequence of P2RX7 (also known as NR_033950). | |
| (SEQ ID NO: 32) | |
| 1 gtcattggag gagcttgaag ttaaagactc ctgctaaaaa ccagtacgtt tcattttgca | 61 |
| gttactggga gggggcttgc tgtggccctg tcaggaagag tagagctctg gtccagctcc | 121 |
| gcgcagggag ggaggctgtc accatgccgg cctgctgcag ctgcagtgat gttttccagt | 181 |
| atgagacgaa caaagtcact cggatccaga gcatgaatta tggcaccatt aagtggttct | 241 |
| tccacgtgat catcttttcc tacgtttgct ttgctctggt gagtgacaag ctgtaccagc | 301 |
| ggaaagagcc tgtcatcagt tctgtgcaca ccaaggtgaa ggggatagca gaggtgaaag | 361 |
| aggagatcgt ggagaatgga gtgaagaagt tggtgcacag tgtctttgac accgcagact | 421 |
| acaccttccc tttgcagggg aactctttct tcgtgatgac aaactttctc aaaacagaag | 481 |
| gccaagagca gcggttgtgt cccgaggaat tcagaccgga aggtgtgtag tgtatgaagg | 541 |
| gaaccagaag acctgtgaag tctctgcctg gtgccccatc gaggcagtgg aagaggcccc | 601 |
| ccggcctgct ctcttgaaca gtgccgaaaa cttcactgtg ctcatcaaga acaatatcga | 661 |
| cttccccggc cacaactaca ccacgagaaa catcctgcca ggtttaaaca tcacttgtac | 721 |
| cttccacaag actcagaatc cacagtgtcc cattttccga ctaggagaca tcttccgaga | 781 |
| aacaggcgat aatttttcag atgtggcaat tcagggcgga ataatgggca ttgagatcta | 841 |
| ctgggactgc aacctagacc gttggttcca tcactgccgt cccaaataca gtttccgtcg | 901 |
| ccttgacgac aagaccacca acgtgtcctt gtaccctggc tacaacttca gatacgccaa | 961 |
| gtactacaag gaaaacaatg ttgagaaacg gactctgata aaagtcttcg ggatccgttt | 1021 |
| tgacatcctg gtttttggca ccggaggaaa atttgacatt atccagctgg ttgtgtacat | 1081 |
| cggctcaacc ctctcctact tcggtctggt aagagattct cttttccatg ctttaggaaa | 1141 |
| atggtttgga gaaggaagtg actaacgcag cgcttgtctg cattctcccc aggccgctgt | 1201 |
| gttcatcgac ttcctcatcg acacttactc cagtaactgc tgtcgctccc atatttatcc | 1261 |
| ctggtgcaag tgctgtcagc cctgtgtggt caacgaatac tactacagga agaagtgcga | 1321 |
| gtccattgtg gagccaaagc cgacattaaa gtatgtgtcc tttgtggatg aatcccacat | 1381 |
| taggatggtg aaccagcagc tactagggag aagtctgcaa gatgtcaagg gccaagaagt | 1441 |
| cccaagacct gcgatggact tcacagattt gtccaggctg cccctggccc tccatgacac | 1501 |
| acccccgatt cctggacaac cagaggagat acagctgctt agaaaggagg cgactcctag | 1561 |
| atccagggat agccccgtct ggtgccagtg tggaagctgc ctcccatctc aactccctga | 1621 |
| gagccacagg tgcctggagg agctgtgctg ccggaaaaag ccgggggcct gcatcaccac | 1681 |
| ctcagagctg ttcaggaagc tggtcctgtc cagacacgtc ctgcagttcc tcctgctcta | 1741 |
| ccaggagccc ttgctggcgc tggatgtgga ttccaccaac agccggctgc ggcactgtgc | 1801 |
| ctacaggtgc tacgccacct ggcgcttcgg ctcccaggac atggctgact ttgccatcct | 1861 |
| gcccagctgc tgccgctgga ggatccggaa agagtttccg aagagtgaag ggcagtacag | 1921 |
| tggcttcaag agtccttact gaagccaggc accgtggctc acgtctgtaa tcccagcgct | 1981 |
| ttgggaggcc gaggcaggca gatcacctga ggtcgggagt tggagacccg cctggctaac | 2041 |
| aaggcgaaat cctgtctgta ctaaaaatac aaaaatcagc cagacatggt ggcatgcacc | 2101 |
| tgcaatccca gctactcggg aggctgaggc acaagaatca cttgaacccg ggaggcagag | 2161 |
| gttgtagtga gcccagattg tgccactgct ctccagcctg ggaggcacag caaactgtcc | 2221 |
| cccaaaaaaa aaaaagagtc cttaccaata gcaggggctg cagtagccat gttaacatga | 2281 |
| catttaccag caacttgaac ttcacctgca aagctctgtg gccacatttt cagccaaagg | 2341 |
| gaaatatgct ttcatcttct gttgctctct gtgtctgaga gcaaagtgac ctggttaaac | 2401 |
| aaaccagaat ccctctacat ggactcagag aaaagagatt gagatgtaag tctcaactct | 2461 |
| gtccccagga agttgtgtga ccctaggcct ctcacctctg tgcctctgtc tccttgttgc | 2521 |
| ccaactacta tctcagagat attgtgagga caaattgaga cagtgcacat gaactgtctt | 2581 |
| ttaatgtgta aagatctaca tgaatgcaaa acatttcatt atgaggtcag actaggataa | 2641 |
| tgtccaacta aaaacaaacc cttttcatcc tggctggaga atgtggagaa ctaaaggtgg | 2701 |
| ccacaaattc tttgacactc aagtccccca agacctaagg gttttatctc ctccccttga | 2761 |
| atatgggtgg ctctgattgc tttatccaaa agtggaagtg acattgtgtc agtttcagat | 2821 |
| cctgatctta agaggctgac agcttctact tgctgtccct tggaactctt gctatcgggg | 2881 |
| aagccagacg ccatttaaaa gtctgcctat cctggccagg tgtggtggct cacacctgta | 2941 |
| atcccagcac tttgggagac caaggcgggc ggatcactta aagtcaggag tccaagacca | 3001 |
| gactcgccaa catggtgaaa ccgtatctct aataaaaata caaaaattag ctgggcatgg | 3061 |
| tgcgggcacc tgtagtccta gctatcaaga ggctgagaca ggagaaacac ttgaacctgg | 3121 |
| gaggtggagg ttgcattgag ctgagatcgt gccactgcac tccaggctgg gtgacagagc | 3181 |
| gagactccat ctcaaaaaaa aaaaaaagaa aaaaaaaatg tctgcctatc ctgagactgc | 3241 |
| cctgctgtga ggaagcccaa gcagtcacgt ggacagtgcc tgaccagccc cagctttcaa | 3301 |
| gccatccaag cccagtcacc aaacatgaga gagaagaagc cttcaggtga ttctggactc | 3361 |
| cactaacata tgactgatac cgcatgatac atcccaagtg agaactgccc cataaatcca | 3421 |
| gaaaaccaca ttgctatctt aagtccctaa gtttggggct tatttgttcc acagcaacag | 3481 |
| gtaactggaa cagagggcaa gcctgatgaa tgggcacaca gactcagccc ataccttccc | 3541 |
| tggttctaat gttctcaggg agcccggacc aaccctggga gcctcaggaa cttaggtttc | 3601 |
| cactggacag ttctagaagg gctatagacc aaatcaggta actcaccaga ccagccttgg | 3661 |
| aatctatcaa atctaactgc tgagctaccc a | |

SEQ ID NO: 33 is the nucleotide sequence of AC010980.2.
(SEQ ID NO: 33)
TTCCTGGGCTCCCTCACTGGCTTCGTCCACTTAAACTTTTAACGTACACATGTAGAGAGCTGAAACTCCAGGGG
TAACATGGGACAGGTCCTCTTGATTTAATGAAAACAGAAGATCAACTGGACCGGGTAGCAAGAAATAAGGCTTA
AGAAGCACTGGGTTGGGCCTCACCCTATGAGCAGGGAACCCACCTTCCCCCTCAACAAAAGAAAAGATGACTTT
GGAATGTTCTGGATTCCCACAGCTGGGAATGTTCCTACTTTTTTTTCAGTTTCTCTGCAGAAGACAGCAAGATG
CCCCAGGGAATGTTTGTGAAAAAGGATGACTGGATGGGAAGCAAGCT
SEQ ID NO: 34 is the nucleotide sequence of RP11-536K7.5.
(SEQ ID NO: 34)
CTTTGAATGTGGCGTGTGGGATCTCTGGCGGGTCATCGTCACAGAGCTCTGCAAAGCAAA
AGAAGCCTATTAGGAACTCAAGAGGCCCCAGGAGTTCTGTATGCAGCTGAAGGGTAACAT
CCCTTGGGAGCTTTTGCCTTGTCTGGCTGACGCCTTGGCAATCGCAAGTGACAGTGACGG
AGAGCCACCTCCTCTGGAATGCATCATGGAACAGGTCTGGGAGAACAGAGGGTGCAGAAA
GGGAAAAGCCAGGCATTTGTAGTCCTCCTGCCACAGATGACCAGAATGACCTCACCCAGG
ACAAACGGCGACAGATTTAGGAATTCAAGCCAGCTTTTCTGACTCCCAGGCACCTGCTCT
CTCCTTTCGAGTGTTCGACTCCACTCTCAGTCACTCGTCACTCTTCCCAGAAGGACAAAT
GTGCAGCTTCTATGTCTTGGATTTCAAATAAAAGACGAGGAACTTGGTGAAATGAGGTGG
SEQ ID NO: 35 is the nucleotide sequence of RP11-58A12.2.
(SEQ ID NO: 35)
GAGTAGAAACACCCCTGTGATACTGTTCTTAATATTCAGGGAGGAAGAGGATGATATTACTCCCAATACAGACG
GGTGTACACCCTCTGTACACCGAGGGTGTACACCTGTCTGTGAAAGAGTTCGTAATCTCCAGAGGGGGAGATGA
TATTACTCACAATATGGTAAAGAGGCTGTGAGTCCACGGAGGATCCTCAGAGCCAGCGGGGGAAGAGGGGCTGG
CTCTCAGTCCCCGCCTCGCGGGGGTGACTCCCCCCAGTGCGATGGGGGTCCTAAGAGCCAGTGGGGGAAGAGGG
GCTGGCTCTGAGACCCCGCCTCGCGGGGGGTGCCTCCCCGCCCTGTGATGGGGGTCCGAAGAGCCAGAAGGCTT
AGAGGGGCTGGCTCTCAGTCCCCGCCTCGCGGGGGGTGCCTCCTCCCCCTGCGATGGGGGTCGTAAGAGCCACT
GGGGGAATAGGGGCTTGCTCTCAGTCACCGCCTCGCGGGGGGTGCCTCCTTTCCTTTCTACATAGACACAGTGA
CAGTCTGATCTCTCTTTCTTTTCCCTACAGATGGACACGCCCCCACTGATATTGTTTTTAATGCAGCGTGGGAG
GGGAGGATATGACACGCGATATCGCAGGTAGTAGAAACACCCCTTTGATAGTGTTCTTAATATTCAGGGAGGAA
GAAGATGATGTTACTCCCAATACAGACGGATGTACACCCTCTGTACACCGAGGGTGTACACCCGTCTGTGAAGG
AGTTCGTAATCTCCAGAGGTGGAGATGATATTACTCACAATATGGTAAACAGGCTGTGAGTCCATCGCGGATCC
TCAGAGCCAGGTGGGGAAGAGGGGCTGGCTGTCAGTCCCCTCCTCGCGGGGGGTGCCTCCCCCACTGCTATGGG
GATCCCAAGAGCCAGTGGGGGAAGAGGTGCTGGCTCTCAGTCTCCGCCTCGCGAGGTGCCTCCCCACCCTGCGA
TCGGGGTCCGAAGAGCCAGGGGGGAAGAGGGGCTGGCTCTCTTCGTGGATGATTCTTTTTCCATTCTCAGGCAG
TTTTCTTTTTTCTTTCTTTCTTTTTTTTTTTTTTTGAGACTGAGTCTTGCTCTGTTGCCCATGCTTTGCTCGAT
CTCGGGTGACTGCAACCACTGCCTCCCAGGTTCAAGAGATTCTCCTGCCTCAGCCTCCTGAGTAGCTGGGACTA
GAGGCGTGTGTCACCACACCCAGCTAATTTTTGTATTTTTAGTAGAGATGGGGTTTCACCATGTTTGCCAGGAT
GGTCTCTATCTCCTGCCCTTGTGATCCACCCACCTCAGCCTCCCAAAGTGCTGGGATTGCAGGTGCGAGCCACC
GGGTCCAGCCTCTCAGGCGATTTTCATACCTGCATACTCTGGTCACTACTCTGTTAAACAGTCAAGGAGGGTAA
GTATTATCTTCAGATTTCCAGAGCTCTGTCTCTGTACAGCCCTCTCCTCCTCAATATTCTGCCCTATGAATTCT
AGCCACATTGGCCTTCCCAGGCTCACAGTTCTGTCTTCTCAACTCAGGAAGATCTCTGAGTTCCATCTGCATTC
TTTCTTCCTGTGCTGTGGCCTGGAAAGTTTTCTAAGGTGTTAGGGAGGTCAATTGTGGGGCTAGCCTCATTTGT
TTCTCATCTCTTGAGGATCACTGCCCTTTGATGCTTGATTCCAGTGATTGATTCCCTTTGTTGCTTGAGGGCCA
TAGTTTCATATATTTTGTCCAGTAGTTTTGTTGTTTTAGGTCAGAAAGTAATTTTGGTCTCTGTTACTCTATCT
TGGCCAGAAGTGTAAGACCTAAGCATTTACACATCAAAATACTGCACACATAATTTTAGTTTAAGCTACTTTTT
AAAAAATCTCCTTCATTTTCCATTTAGCATTCTATTTAGGGTATTACATTGGTTTTTTTGAAATTCTGTTATTG
GCAGTTTCTATTGCCTATCAATCCCATTTAAAGATAGTGCATAGGGTATTCTAAAATAGCTGTTAAGCAAAGAG
AAAATTGGGCCTGATAGGGTGAGAATCACAGCTCTAATACCTAGAGTGACCTTATAATGTATTGTCCAAAGGAG
ATATTTTTGACAGTGAAAGAGGGTGTTGTTAGTAATTATATCAGGACCATGGCCTAAACCAGGACTATCCCAGG
AAGCCTGGGACATATTTGTACCCCATCTCTATTTAATGCCTTTATACAATTCTTTACTTAATTCTACCAGCCTT
TATTGAGCCTGCTTTCTTTGTCTAGCTGAGTGCCACGTGCTGACGTCACTAAGATCAATACAGCAAACTCTGAA
AGATGGACAGAGAGACAGGAGATGGTCCTTTATAATGCAGTGTGATCTGTGCTGCAATAGAGATGGAAGAGTGT
TCTGATCGGCTGGAAAACAACGCACATGGGAAGCTGTACAGAAATGAGTGGGGAAAG
SEQ ID NO: 36 is the nucleotide sequence of AL078621.3.
(SEQ ID NO: 36)
CTGACCTTGTGATCCACCCACCTCAGCCTCCCAAAGTGCTGGGATTGCAGGTGGGAGCCACCGGGTCCAGCCTC
TCCGGCGATTTTCATACCTGCATACTCTGATCACTACTCTGTTAAACAGTCAAGGAGGCTGAGTGCCACGTGCT
GACGTCACTAAGATCAATACAGCAAACTCTGAAAGATGGACAGAGAGACAGGAGATGGTCCTTTATAATGCAGT
GTGATCTGTGCTGCAATAGAGTTTTGGAAGCTAGAAGTTCAAAACGAGGTGTTGGCAGCACCATGCTCTCTCTG
AAGATGCTAGGAAGAATCTGCTCCATGCCTTTCCATTCGCTCCTGGGGTTTCCTGCAAGCCCTGACATTCCTTG
GCTTGTAGATACACCACCCCAGTTTCCGCCCCCATCATCACATGGCCTCCTCTCTGTGGGTGCCTCTGCGTTCC
CTCTATTCTTCTTCTAAGGACACCGACACCAGTCATAGTGGATTAAGGGTCCACTCCTAACTAATTACATCTGC
AACCACCCTATTTCCAAATAAAGTCACATTCTAAGATTCCTAGGGAGAACATGAAGTTTTGGGGGTGTGTGGAT
ACTGTTCAACCTGGGACATGGAGTAAATAAATGGCAAGAAAGATTACAGATGACTTTAAGCTAAAGAGGGAGAT
AGGGTTAAATGTAGGACTTTTTTTTTTTTCCAGGACGGGAGAGGTTTAAACATGTTACTACATTGAATAAATGA
AATAA
SEQ ID NO: 37 is the nucleotide sequence of RP13-452N2.1.
(SEQ ID NO: 37)
GACGGGCAGACCACCTGAGCTCAGGAGCTCAAGACCAGCCTGGGCAACATGGCAAAACCCTGTCTCTACTAACA
CTATAAAAAGTTAGCTGGGCGTGTTGGTGCATGCTGTAGTCCCAGCTACTCAGGAGGCTGAGCCACGAGAATCG
CTTGAACCTGGGAGATGGAGGTTGAAGTGAGCCAAGATCGCACCATTGCACTCCAGCCTGGGGAAAAAAAAAAG
CCAGGCCTCCTAATGTACTCATTTAAACATCACTTGTGTCCACCTGGGTTCAGAATTGCGCTGGACATGGGATA
CACAAAGCGGGAGTGGAATTTTCAGGAAGCAGCTGTGCTGTGGGTGACGGTCATACTTTATTCTCTAAAACTCT
CCAGTTTCAATTGGCCTCTTGGTGCCAAAGCACCATGGCGTGTTACACCTATCAGATCGGGCACACTGCCCCTT
CTTCCCAACCTACGAGCTGAAATGGCCAGCACTGCGCCTTCCAGAACCCAGCGTAGATGCACAGCTGCCACGGA
GGTAGGAAGAATGATTCGTTTGCCACCAAAAGCCCAGTTTCTCCAACAGGTGAATCCCTAGGCCAGCCCGGCCC
CTTCCCACACACCCCTACCCCGAGACGGGTGCAGAATCAGCAGCGGGGATCATCCAGAGACTCTCGCAGTCCAC
GGCAGGTGTCGGAGTGGGCCTCATTCCAGAGTTGTGGTGGCCGCTCCACCTGGCGAGCTTCGAGGGGACTCCGG
CAGGCGCTCGCTGTGGGGTCCTCCCACCAGTCTTCTCAGCACAGGAACGGTGCCTTCACATTGGTCTTCCGGGG
CTTAAAAGCATATCATTCCTGCTTGCTCTTCAGATGCAAACTCCCCCTCCAAGGATACCTCCAGGTTCCCTGCC
AGCTCACTTGACTCACATGGCAATGGTCACCGGGCTGCCTGAGCCCAAATCAAGCACCGTTTATTAGGGGGAAG
CTCCGTTATCCAGAAAGCTCATCTTTGAAGAGCTACCCATTTCCTGCCAAAGTTGTGGCGGGAAGGCCTTAGGT
ACTAGAAAACCTTGGGGAATGGCAATTAAAGGATTATCATTCTTACTTATTTGTTTGGATAAGCAGCAATCACC
CTTCGGATGCTTATTGGTGAGAATATTTCTAAAAGTGTCCTGCACATTTCCCTGCTTTGATGAATGGAACGGCA
GGGCAGGATTTAACTGTCTGATGGCTACGGTTTTGGGAAAGTGCCAGAACCTCCACGTCAGCCACCCCAGAGCC
ACAGGCAGGGTATGAGCCACAGCAATGGGCCCCAAGAAAGAGCCCCACACCCACCATGGTCCACGGAAGACGCT
GCTTACTGGCCCCGGGTTACCTGGAGTCATGTGCATTCTCCTTACATCAAGTGAAGATAATATTTTGTTTCCTT
CCCACTTTTTCTCTAACACAAGCCACCCCAGTTCTCCCTTCTGACAACAGGCAAGTGGACCCTAGTAGACCTTC
CAGAAAAGCCTTAGAGAGCACCCCCAAATGCTCACATTTGTCAGCTGTAATTCCACCCTCAGTTAAAACCTGAT
TCTCAATGTGACTTGGCCAACGCAGCTTGCAAAGCAAGAACGTCTTCATCTAAGGTTGAAAACATCAGTTGTTT
GGGTTGTGAGAAAAGAAAAGCCCACAAGGTTTCCGAGGCTTGGTGCTGTTTCTCACTGTCCTCAGCTCCTGATT
CTGCAGCTGGTTCTCTCTCCAAGCACTAGCAAAACCCTGCCCTAGGAGCCCCCAGACTCTGAGAGCCCATGACC
AAAAAGAAAAGGAAAGCCAAGTTGGGGAAGAACAGGGGCCCCCAACTCCACAGCCCTCCACTCTGCCCAGAGGG
CCCACCCTGGGCTGCCTGGAACCCCCTAAAGTTGCCACCCCCGCAACACAGTAGTGGGGCAGTTCCTGGCAGCG
CCTGCAGCCCATGGGCTGGCTCTGTACCCGCAGCCCCGCCAAGCGTCTGTTATCTTATTTACTGGAATCTGCAC
AGCCAGGCTCTAGCTCACCGGTGACTAAGGAGCTGCAGCCATTATTACCAGGCAGATGGCAGACTCCCTAAAAG
CAGACATTAAACAATAAAATGCCACCACATACCTTGCCCACAAAATAAAATCAAAACAAACACCTAA

What is claimed is:

1. A method of treating or preventing an inflammatory disease in a subject, the method comprising: administering to a subject an effective amount of an agent that inhibits a long noncoding RNA(lnc-RNA) expressed in a macrophage, wherein the lnc-RNA is inhibited in a target cell.

2. The method of claim 1, wherein the lnc-RNA is selected from the group consisting of: AL078621.3; RP11-536K7.5; RP11-143M1.3; RP11-561O23.5; AC10980.2; P2RX7; LOC440896; D63785; RP11-58A12.2; RP11-79H23.3 (lnc-FAM164A1); and RP13-452N2.1.

3. (canceled)

4. The method of claim 1, wherein the inflammatory disease is selected from the group consisting of: endotoxemia, atherosclerotic vascular disease, coronary artery disease, peripheral arteru disease, in-stent restenosis, vein graft disease, arteriovenous fistula disease, vascular calcification, heart valve disease, heart failure, autoimmune disease, osteoporosis, ectopic calcification, brain damage after stroke, obesity, fatty liver disease, diabetes, Gaucher's disease, sepsis, kidney dysfunction, kidney failure, and cancer.

5. The method of claim 4, wherein the heart valve disease is aortic valve disease or mitral valve disease.

6. The method of claim 1, wherein the inflammatory disease is acute or chronic, or occurs in response to a tissue or cell transplantation.

7. (canceled)

8. The method of claim 1, wherein the agent is selected from the group consisting of: a small molecule, an antibody, a peptide, a genome editing system, an antisense oligonucleotide, and an RNA interference (RNAi), an antisense RNA, an RNA decoy molecule, an RNAaptamer, and an inhibitory polypeptide.

9. (canceled)

10. The method of claim 8, wherein the agent is a vector that encodes the agent.

11. (canceled)

12. The method of claim 1, wherein the non integrative vector is selected from the group consisting of an EBNA1 vector, a minicircle vector, a non-integrative adenovirus, a non-integrative RNA, and a Sendai virus, an episomal vector, and a lentiviral vector.

13.-17. (canceled)

18. The method of claim 15 or 16, wherein the target cell is a macrophage, an activated macrophage, a T cell, a dendritic cell, a B cell, a natural killer cell, or a neutrophil.

19.-22. (canceled)

23. The method of claim 1, wherein the effective amount of the agent reduces the level or activity of lnc-RNA by at least 50%, at least 60%, at least 70%, at least 80%, at least 90%, or more as compared to an appropriate control.

24.-26. (canceled)

27. The method of claim 1, wherein the subject has previously been diagnos[[is]]ed with having an inflammatory disease, has not been previously been diagnosed with having an inflammatory disease, or exhibits at least one risk factor of developing an inflammatory disease.

28.-30. (canceled)

31. The method of claim 1, further comprising, prior to administering, diagnosing a subject as having an inflammatory disease; or prior to administering, receiving the results of an assay that diagnoses a patient as having an inflammatory disease.

32. The method of claim 1, wherein the agent decreases at least one of
a. expression of CCL2 and IL-6 in a plasma cell and/or a peritoneal cell, or
b. decreases NFkB signaling.

33. (canceled)

34. A method of treating or preventing an inflammatory disease in a subject, the method comprising: administering to a subject an effective amount of an agent that inhibits a protein bound to a lnc-RNA.

35. The method of claim 34, wherein the protein bound to the lnc-RNA is selected from the group consisting of: ATP-citrate synthase (ACLY); Filaggrin-2 (Flg2); and Splicing factor, proline-and-glutamine-rich (SFPQ).

36. (canceled)

37. The method of any of claim 34, wherein the agent decreases the expression of CCL2 and TNF-α in the subject.

38.-40. (canceled)

41. The method of claim 34, wherein the effective amount of the agent reduces the level, activity, or binding of the protein by at least 50%, at least 60%, at least 70%, at least 80%, at least 90%, or more as compared to an appropriate control.

42. (canceled)

43. (canceled)

44. A method of treating an inflammatory disease in a subject, the method comprising:
a. measuring a level of a lnc-RNA expressed in a macrophage in a biological sample or receiving results of an assay that measures a level of a lnc-RNA expressed in a macrophage in a biological sample;
b. comparing the level of the lnc-RNA to a reference level;
c. identifying a subject as having an inflammatory disease if the level of the lnc-RNA is increased as compared to the reference level; and
d. administering to the subject having an inflammatory disease an agent that inhibits the lnc-RNA, or a composition comprising an agent that inhibits the lnc-RNA.

45.-47. (canceled)

48. The method of claim 4, further comprising, prior to step a), obtaining a biological sample from the subject.

49. A pharmaceutical composition comprising an agent that inhibits a lnc-RNA expressed in a macrophage, or an agent that inhibits a protein bound to a lnc-RNA and a pharmaceutically acceptable carrier.

50.-57. (canceled)


SEQUENCE LISTING (58 sequences)

SEQ ID NO: 1 - DNA, 2994 bp, Unknown
  Description of Unknown lnc-FAM164A1 sequence
tctaaatcccatttctattctattattattactactacaaaaatagtaataaaaacaaacatttaaataatatctaataaataccaactattcttctaagaacttgacatgtataaactcatttaagcttcaactctgtgaaatagggtatcagttatgtattgccaaaataacgctatatagcaaactaccccaaaacataaaactgtaaaaacataattacaacaatagacctttttatcctttctcacaagccactgtttttgctgatctgggccaagttcaaaagacattgacttggcccactcacctgtggacagtcagctgccttgttggatgggtgctgggagtataagatggcctcagttaggactgcttggctgttctctagaagttctctcattttcacaatgctagcctggacttatttccattgccataggagggttctaacagagagaaagagagcggaagcaggcaatgcttcttaaagcctaggtttggaatgcacactgttacttcccacgtatggtattggacagagcaagtgaaaacacctagtgccaagagtatgcatccaggaaggggtaaagaacaggactagttttgccatttattaaggtattattattaatccttattttagaagaaactgatatatagaggagttaagcaacttgctcaaggtcgtggactaatgaaacaatggcactagtatttaaatactgtcagtctggtttcattctgtgctgttaacttctacatggtgctgtcaacttctacatggtgctgtctctcagctgaaggaacgatagaggaggaaaggcaaaaggcaacaatgggctttgaagtaactctactgagataattaaaagaccacataaaagaaggaaaacgtatcttacttcaggaattcaacactaagaatcctactcccttcgtgaaatcagaaaaggtttgcatgaaccctgtgggcctttgaaagagagaaggcatactgttgatggtcaccaacttactgaagcctgcagcttgcaggaacccatctcatcagagtaactgaaagagaacacagtatccaaaaacaaaaacaacaaaagtcacgtgctgggataagttcttgatagtgtgaatagtcagaaaaaaatgttctgaaacttcttgtcccagctgcgttattaactgtgaatttcatcatatggttgattaagttgctttataagactaaatttttataagatgtgtacataggaatatgacttccctctcctaagttaacataacaggtattcccaaatgtgttgtgaagtcgacattcttgttctaatttatagttgtattgctccctgcaatagcatcttctttcatttcccagtctccagtttttccattttgtttacagtagccagagaaatctatcaagatgcacacttgatcacagcatccttcggtaaatcaccattatcttcatactgacaaaggtcagtctttaaggacctttatcaggtgtcctctctttagttccccaacctatttcttaccatcctttatcccccagctaaataacttggagaattccaagtgcgtcatccactgtattagtctgttctcacatcgctaacaaagacatacctgagactggggaatttataaaggaaagaggtttaatggactcacagttccgcagggctagggaggcttcacaatcatggcagaagacaaagcaagagcaaagggaagtcttacatggcagcaggcaagagagcttgtgcagggggaactcctgtttataaaaccatcagatcttgggagatgtgttcaatacgatgagaacagtgtgggggaaaccacccccatgatgccattatctccacttgtctctgtccttgacacatgggaattattataattctaggtgagatttgggtggggacacagagccaaacaatatcatccaccttcatgtctctttctctttactcacctgctttcttgacaagaaatatcttcatcagcccttccttccttccctccctccctcctttctttctttcttgcttgcttgcttttccttcctttttcttctttatttcctttgccaatttgagagattggcaaggagagtaatgctggaattggagacacatataactaataccagccaaaataattgtgagcagaagtgatatgccttctgagccaaggcagagaaaagattatatgtgattctcagacctcttctcatcctcattggctaagaagggcataggtcccagatggaacagctatacgttaatggggccttcctccatttggatccctaagtaactgaaatggagcccctctccaacctgtgataaaattgtgctacaaaggagaactcaattttcttgtgctaaaatactgagatttggaaattaatgtgtgctgcattataacctaggttattataaagatattattgaaaattacttttaatattcatttttgtcattattggtgaatttgaagtttcatataattattgaacacttatatgtcatttatatatttattatacttagttctttggcaaaatgctgtttatgtcctttgcctatttttctactagactgtttatgttgatttataaaacatatttatatattaggctacagtaaccctttcatgtcattggtactgtttccaatttattaactgccttttattatcactttatgatgtattttatgtccaaaggtttaaaaattatatactgagtaatcgataattatttaaaaattttgttcttattttatgagaaaggccttctaaaatctgataggttggtttttctctggatatactattttctattttttaaaacatgaaattctatactccattatgatttatgttgttgcatgctatctggtcggaatatagctttgtaatgaatacattcgtcttaaaactgacttggctttttctcctaaattaaaaagacaactctga

SEQ ID NO: 2 - protein, 1101 aa, Unknown
  Description of Unknown ACLY sequence
MSAKAISEQTGKELLYKFICTTSAIQNRFKYARVTPDTDWARLLQDHPWLLSQNLVVKPDQLIKRRGKLGLVGVNLTLDGVKSWLKPRLGQEATVGKATGFLKNFLIEPFVPHSQAEEFYVCIYATREGDYVLFHHEGGVDVGDVDAKAQKLLVGVDEKLNPEDIKKHLLVHAPEDKKEILASFISGLFNFYEDLYFTYLEINPLVVTKDGVYVLDLAAKVDATADYICKVKWGDIEFPPPFGREAYPEEAYIADLDAKSGASLKLTLLNPKGRIWTMVAGGGASVVYSDTICDLGGVNELANYGEYSGAPSEQQTYDYAKTILSLMTREKHPDGKILIIGGSIANFTNVAATFKGIVRAIRDYQGPLKEHEVTIFVRRGGPNYQEGLRVMGEVGKTTGIPIHVFGTETHMTAIVGMALGHRPIPNQPPTAAHTANFLLNASGSTSTPAPSRTASFSESRADEVAPAKKAKPAMPQDSVPSPRSLQGKSTTLFSRHTKAIVWGMQTRAVQGMLDFDYVCSRDEPSVAAMVYPFTGDHKQKFYWGHKEILIPVFKNMADAMRKHPEVDVLINFASLRSAYDSTMETMNYAQIRTIAIIAEGIPEALTRKLIKKADQKGVTIIGPATVGGIKPGCFKIGNTGGMLDNILASKLYRPGSVAYVSRSGGMSNELNNIISRTTDGVYEGVAIGGDRYPGSTFMDHVLRYQDTPGVKMIVVLGEIGGTEEYKICRGIKEGRLTKPIVCWCIGTCATMFSSEVQFGHAGACANQASETAVAKNQALKEAGVFVPRSFDELGEIIQSVYEDLVANGVIVPAQEVPPPTVPMDYSWARELGLIRKPASFMTSICDERGQELIYAGMPITEVFKEEMGIGGVLGLLWFQKRLPKYSCQFIEMCLMVTADHGPAVSGAHNTIICARAGKDLVSSLTSGLLTIGDRFGGALDAAAKMFSKAFDSGIIPMEFVNKMKKEGKLIMGIGHRVKSINNPDMRVQILKDYVRQHFPATPLLDYALEVEKITTSKKPNLILNVDGLIGVAFVDMLRNCGSFTREEADEYIDIGALNGIFVLGRSMGFIGHYLDQKRLKQGLYRHPWDDISYVLPEHMSM

SEQ ID NO: 3 - DNA, 3306 bp, Unknown
  Description of Unknown ACLY sequence
atgtcggccaaggcaatttcagagcagacgggcaaagaactcctttacaagttcatctgtaccacctcagccatccagaatcggttcaagtatgctcgggtcactcctgacacagactgggcccgcttgctgcaggaccacccctggctgctcagccagaacttggtagtcaagccagaccagctgatcaaacgtcgtggaaaacttggtctcgttggggtcaacctcactctggatggggtcaagtcctggctgaagccacggctgggacaggaagccacagttggcaaggccacaggcttcctcaagaactttctgatcgagcccttcgtcccccacagtcaggctgaggagttctatgtctgcatctatgccacccgagaaggggactacgtcctgttccaccacgaggggggtgtggacgtgggtgatgtggacgccaaggcccagaagctgcttgttggcgtggatgagaaactgaatcctgaggacatcaaaaaacacctgttggtccacgcccctgaagacaagaaagaaattctggccagttttatctccggcctcttcaatttctacgaggacttgtacttcacctacctcgagatcaatccccttgtagtgaccaaagatggagtctatgtccttgacttggcggccaaggtggacgccactgccgactacatctgcaaagtgaagtggggtgacatcgagttccctccccccttcgggcgggaggcatatccagaggaagcctacattgcagacctcgatgccaaaagtggggcaagcctgaagctgaccttgctgaaccccaaagggaggatctggaccatggtggccgggggtggcgcctctgtcgtgtacagcgataccatctgtgatctagggggtgtcaacgagctggcaaactatggggagtactcaggcgcccccagcgagcagcagacctatgactatgccaagactatcctctccctcatgacccgagagaagcacccagatggcaagatcctcatcattggaggcagcatcgcaaacttcaccaacgtggctgccacgttcaagggcatcgtgagagcaattcgagattaccagggccccctgaaggagcacgaagtcacaatctttgtccgaagaggtggccccaactatcaggagggcttacgggtgatgggagaagtcgggaagaccactgggatccccatccatgtctttggcacagagactcacatgacggccattgtgggcatggccctgggccaccggcccatccccaaccagccacccacagcggcccacactgcaaacttcctcctcaacgccagcgggagcacatcgacgccagcccccagcaggacagcatctttttctgagtccagggccgatgaggtggcgcctgcaaagaaggccaagcctgccatgccacaagattcagtcccaagtccaagatccctgcaaggaaagagcaccaccctcttcagccgccacaccaaggccattgtgtggggcatgcagacccgggccgtgcaaggcatgctggactttgactatgtctgctcccgagacgagccctcagtggctgccatggtctaccctttcactggggaccacaagcagaagttttactgggggcacaaagagatcctgatccctgtcttcaagaacatggctgatgccatgaggaagcatccggaggtagatgtgctcatcaactttgcctctctccgctctgcctatgacagcaccatggagaccatgaactatgcccagatccggaccatcgccatcatagctgaaggcatccctgaggccctcacgagaaagctgatcaagaaggcggaccagaagggagtgaccatcatcggacctgccactgttggaggcatcaagcctgggtgctttaagattggcaacacaggtgggatgctggacaacatcctggcctccaaactgtaccgcccaggcagcgtggcctatgtctcacgttccggaggcatgtccaacgagctcaacaatatcatctctcggaccacggatggcgtctatgagggcgtggccattggtggggacaggtacccgggctccacattcatggatcatgtgttacgctatcaggacactccaggagtcaaaatgattgtggttcttggagagattgggggcactgaggaatataagatttgccggggcatcaaggagggccgcctcactaagcccatcgtctgctggtgcatcgggacgtgtgccaccatgttctcctctgaggtccagtttggccatgctggagcttgtgccaaccaggcttctgaaactgcagtagccaagaaccaggctttgaaggaagcaggagtgtttgtgccccggagctttgatgagcttggagagatcatccagtctgtatacgaagatctcgtggccaatggagtcattgtacctgcccaggaggtgccgcccccaaccgtgcccatggactactcctgggccagggagcttggtttgatccgcaaacctgcctcgttcatgaccagcatctgcgatgagcgaggacaggagctcatctacgcgggcatgcccatcactgaggtcttcaaggaagagatgggcattggcggggtcctcggcctcctctggttccagaaaaggttgcctaagtactcttgccagttcattgagatgtgtctgatggtgacagctgatcacgggccagccgtctctggagcccacaacaccatcatttgtgcgcgagctgggaaagacctggtctccagcctcacctcggggctgctcaccatcggggatcggtttgggggtgccttggatgcagcagccaagatgttcagtaaagcctttgacagtggcattatccccatggagtttgtgaacaagatgaagaaggaagggaagctgatcatgggcattggtcaccgagtgaagtcgataaacaacccagacatgcgagtgcagatcctcaaagattacgtcaggcagcacttccctgccactcctctgctcgattatgcactggaagtagagaagattaccacctcgaagaagccaaatcttatcctgaatgtagatggtctcatcggagtcgcatttgtagacatgcttagaaactgtgggtcctttactcgggaggaagctgatgaatatattgacattggagccctcaatggcatctttgtgctgggaaggagtatggggttcattggacactatcttgatcagaagaggctgaagcaggggctgtatcgtcatccgtgggatgatatttcatatgttcttccggaacacatgagcatgtaa

SEQ ID NO: 4 - DNA, 21 bp, Artificial Sequence
  Description of Artificial Sequence Synthetic oligonucleotide
  Description of Combined DNA/RNA Molecule Synthetic oligonucleotide
ucgaaguacucagcguaagtt

SEQ ID NO: 5 - DNA, 21 bp, Artificial Sequence
  Description of Artificial Sequence Synthetic oligonucleotide
  Description of Combined DNA/RNA Molecule Synthetic oligonucleotide
ucaccauuaucuucauacutt

SEQ ID NO: 6 - DNA, 21 bp, Artificial Sequence
  Description of Artificial Sequence Synthetic oligonucleotide
  Description of Combined DNA/RNA Molecule Synthetic oligonucleotide
aguaugaagauaauggugatt

SEQ ID NO: 7 - DNA, 21 bp, Artificial Sequence
  Description of Artificial Sequence Synthetic oligonucleotide
  Description of Combined DNA/RNA Molecule Synthetic oligonucleotide
ugugcuaaaauacugagautt

SEQ ID NO: 8 - DNA, 21 bp, Artificial Sequence
  Description of Artificial Sequence Synthetic oligonucleotide
  Description of Combined DNA/RNA Molecule Synthetic oligonucleotide
aucucaguauuuuagcacatt

SEQ ID NO: 9 - DNA, 15 bp, Artificial Sequence
  Description of Artificial Sequence Synthetic probe
gttaggactgcttgg

SEQ ID NO: 10 - DNA, 21 bp, Artificial Sequence
  Description of Artificial Sequence Synthetic primer
gatgggtgctgggagtataag

SEQ ID NO: 11 - DNA, 22 bp, Artificial Sequence
  Description of Artificial Sequence Synthetic primer
ccgctctctttctctctgttag

SEQ ID NO: 12 - RNA, 19 nt, Artificial Sequence
  Description of Artificial Sequence Synthetic oligonucleotide
ggaugggugcugggaguau

SEQ ID NO: 13 - RNA, 19 nt, Artificial Sequence
  Description of Artificial Sequence Synthetic oligonucleotide
guauaagauggccucaguu

SEQ ID NO: 14 - RNA, 19 nt, Artificial Sequence
  Description of Artificial Sequence Synthetic oligonucleotide
agggguaaagaacaggacu

SEQ ID NO: 15 - RNA, 19 nt, Artificial Sequence
  Description of Artificial Sequence Synthetic oligonucleotide
gcugggauaaguucuugau

SEQ ID NO: 16 - RNA, 19 nt, Artificial Sequence
  Description of Artificial Sequence Synthetic oligonucleotide
auuaucuucauacugacaa

SEQ ID NO: 17 - RNA, 19 nt, Artificial Sequence
  Description of Artificial Sequence Synthetic oligonucleotide
uuaucuucauacugacaaa

SEQ ID NO: 18 - RNA, 19 nt, Artificial Sequence
  Description of Artificial Sequence Synthetic oligonucleotide
caguaacccuuucauguca

SEQ ID NO: 19 - RNA, 19 nt, Artificial Sequence
  Description of Artificial Sequence Synthetic oligonucleotide
uaugauuuauguuguugca

SEQ ID NO: 20 - RNA, 19 nt, Artificial Sequence
  Description of Artificial Sequence Synthetic oligonucleotide
auacucccagcacccaucc

SEQ ID NO: 21 - RNA, 19 nt, Artificial Sequence
  Description of Artificial Sequence Synthetic oligonucleotide
aacugaggccaucuuauac

SEQ ID NO: 22 - RNA, 19 nt, Artificial Sequence
  Description of Artificial Sequence Synthetic oligonucleotide
aguccuguucuuuaccccu

SEQ ID NO: 23 - RNA, 19 nt, Artificial Sequence
  Description of Artificial Sequence Synthetic oligonucleotide
aucaagaacuuaucccagc

SEQ ID NO: 24 - RNA, 19 nt, Artificial Sequence
  Description of Artificial Sequence Synthetic oligonucleotide
uugucaguaugaagauaau

SEQ ID NO: 25 - RNA, 19 nt, Artificial Sequence
  Description of Artificial Sequence Synthetic oligonucleotide
uuugucaguaugaagauaa

SEQ ID NO: 26 - RNA, 19 nt, Artificial Sequence
  Description of Artificial Sequence Synthetic oligonucleotide
ugacaugaaaggguuacug

SEQ ID NO: 27 - RNA, 19 nt, Artificial Sequence
  Description of Artificial Sequence Synthetic oligonucleotide
ugcaacaacauaaaucaua

SEQ ID NO: 28 - DNA, 241 bp, Homo sapiens
tgcccttgctgtcctcctctgcaccatggctctctgcaaccagttctctgcatcacttgctgctgacacgccgaccgcctgctgcttcagctacacctcccggcagattccacagaatttcatggctgactactttgagacgagcagccagtgctccaagcccagtgtcatcttcctaaccaagagaggccggcaggtctgtgctgaccccagtgaggagtgggtccagaaatacgtcagt

SEQ ID NO: 29 - DNA, 617 bp, Unknown
  Description of Unknown RP11-56023.5 sequence
ctgagtgccacgtgctgacgtcactaagatcaatacagcaaactctgaaagatggacagagagacaggagatggtcctttataatgcagtgtgatctgtgctgcaatagagttttggaagctagaagttcaaaacgaggtgttggcagcaccatgctctctctgaagatgctaggaagaatctgctccatgcctttccattcgctcctggggtttcctgcaagccctgacattccttggcttgtagatgcatcaccccagtttccgcccccatcatcacatggcctcctctctgtgtgtgcctctgcgttccctctattcttcttctaaggacaccgacaccagtcatagtggattaagggtccactcctaaccaattacatctgcaacaaccctatttccaaataaagtcacattctaagattcctagggagaacctgaatttttgggggtgtgtggatactgttcaacctgggatatggagtaaataaatggcaaggaagattacagatgactttaagctaaagagggagatagggttaaatgtaggactttttttttttccaggacgggagaggtttaaacatgttactacattgaataaatgaaataatcacg

SEQ ID NO: 30 - DNA, 612 bp, Unknown
  Description of Unknown RP11-143M1.3 sequence
ctgagtgccacgtgctgacatcactaagatcaatacagcaaactctgaaagatggacggagagacaggagatggtcctttataatgcagtgtgatctgtgctgcaatagagttttggaagctagaagttcaaaacgaggtgttggcagcaccatgctctctctgaagatgctaggaagaatctgctccatgcctttccattcgcttctggggtttcctgcaagccctgacattccttggcttgtagatacaccaccccagtttccgcccccatcatcacatggcctcctctctgtgtgtgcctctgcgttccctctattcttcttctaaggacaccgacaccagtcatagtggattaagggtccactcctaaccaattacatctgcaacaaccctatttccaaataaagtcacattctaagattcctagggagaacaggaatttttgggggtgtgtggatactgttcaacctgggacatggagtaaataaatggcaaggaagattacagatgactttaagctaaagagggagatagggttaaatgtaggactttttttttttccaggacgggagaggtttaaacatgttactacattgaataaatgaaataa

SEQ ID NO: 31 - DNA, 3182 bp, Homo sapiens
aaagtttgggattcccagtctacaaaaccccacagctcctaggaattctctcaccacccttgtgcctttaggcttcggtagattgcaaatgacctgctttctttcggatcccgggctgctttcggacacctgtcgaatagtaaatcccaagtaaggtacctgcggtcgtcggcagatctgaattttcttcttggacacctaatacccacagtcctccagagaccgagaggttgatgtcactcccaatatcggaggaagtatacacccccgtgtgagatggtccttaataatattccacggcggagggggtgatatgactacatacatggcagaaagtggaaacctcccagggatattgttcccacgatcctggagggaagaagatgatgttactttcaatatgacagaaggtggatgaagtggtggactgcccctccacacctgtggacacgcccccactgatattccttctaattgcagcgtgggagaggaggatatgacacgcgatatcgcagggagtagaaacacccctgtgatactgttcttaatattcagggaggaagaggatgatattactcccaatacagacgggtgtacaccctctgtacaccgagggtgtacacctgtctgtgaaagagttcgtaatctccagagggggagatgatattactcacaatatggtaaagaggctgtgagtccacggaggatcctcagagccagcgggggaagaggggctggctctcagtccccgcctcgcgggggtgactccccccagtgcgatgggggtcctaagagccagtgggggaagaggggctggctctgagaccccgcctcgcggggggtgcctccccgccctgtgatgggggtccgaagagccagaaggcttagaggggctggctctcagtccccgcctcgcggggggtgcctcctccccctgcgatgggggtcgtaagagccactgggggaataggggcttgctctcagtcaccgcctcgcggggggtgcctcctttcctttctacatagacacagtgacagtctgatctctctttcttttccctacagatggacacgcccccactgatattgtttttaatgcagcgtgggaggggaggatatgacacgcgatatcgcaggtagtagaaacacccctttgatagtgttcttaatattcagggaggaagaagatgatgttactcccaatacagacggatgtacaccctctgtacaccgagggtgtacacccgtctgtgaaggagttcgtaatctccagaggtggagatgatattactcacaatatggtaaacaggctgtgagtccatcgcggatcctcagagccaggtggggaagaggggctggctgtcagtcccctcctcgcggggggtgcctcccccactgctatggggatcccaagagccagtgggggaagaggtgctggctctcagtctccgcctcgcgaggtgcctccccaccctgcgatcggggtccgaagagccaggggggaagaggggctggctctcttcgtggatgattctttttccattctcaggcagttttcttttttctttctttctttttttttttttttgagactgagtcttgctctgttgcccatgctttgctcgatctcgggtgactgcaaccactgcctcccaggttcaagagattctcctgcctcagcctcctgagtagctgggactagaggcgtgtgtcaccacacccagctaatttttgtatttttagtagagatggggtttcaccatgtttgccaggatggtctctatctcctgcccttgtgatccacccacctcagcctcccaaagtgctgggattgcaggtgcgagccaccgggtccagcctctcaggcgattttcatacctgcatactctggtcactactctgttaaacagtcaaggagggtaagtattatcttcagatttccagagctctgtctctgtacagccctctcctcctcaatattctgccctatgaattctagccacattggccttcccaggctcacagttctgtcttctcaactcaggaagatctctgagttccatctgcattctttcttcctgtgctgtggcctggaaagttttctaaggtgttagggaggtcaattgtggggctagcctcatttgtttctcatctcttgaggatcactgccctttgatgcttgattccagtgattgattccctttgttgcttgagggccatagtttcatatattttgtccagtagttttgttgttttaggtcagaaagtaattttggtctctgttactctatcttggccagaagtgtaagacctaagcatttacacatcaaaatactgcacacataattttagtttaagctactttttaaaaaatctccttcattttccatttagcattctatttagggtattacattggtttttttgaaattctgttattggcagtttctattgcctatcaatcccatttaaagatagtgcatagggtattctaaaatagctgttaagcaaagagaaaattgggcctgatagggtgagaatcacagctctaatacctagagtgaccttataatgtattgtccaaaggagatatttttgacagtgaaagagggtgttgttagtaattatatcaggaccatggcctaaaccaggactatcccaggaagcctgggacatatttgtaccccatctctatttaatgcctttatacaattctttacttaattctaccagcctttattgagcctgctttctttgtctagctgagtgccacgtgctgacgtcactaagatcaatacagcaaactctgaaagatggacagagagacaggagatggtcctttataatgcagtgtgatctgtgctgcaatagagttttggaagctagaagttcaaaacgaggtgttggcagcaccatgctctctctgaagatgctaggaagaatctgctccatgcctttccattcgcttctggggtttcctgcaagccctgacattccttggcttgtagatgcatcaccccagtttccgcccccatcatcacatggcctcctctctgtgtgtgcctctgcgttccctctattcttcttctaaggacaccgacaccagtcatagtggattaagtgtccactcctaaccaattacatctgcaacaaccctatttccaaataaagtcacattctaag

SEQ ID NO: 32 - DNA, 3691 bp, Homo sapiens
gtcattggaggagcttgaagttaaagactcctgctaaaaaccagtacgtttcattttgcagttactgggagggggcttgctgtggccctgtcaggaagagtagagctctggtccagctccgcgcagggagggaggctgtcaccatgccggcctgctgcagctgcagtgatgttttccagtatgagacgaacaaagtcactcggatccagagcatgaattatggcaccattaagtggttcttccacgtgatcatcttttcctacgtttgctttgctctggtgagtgacaagctgtaccagcggaaagagcctgtcatcagttctgtgcacaccaaggtgaaggggatagcagaggtgaaagaggagatcgtggagaatggagtgaagaagttggtgcacagtgtctttgacaccgcagactacaccttccctttgcaggggaactctttcttcgtgatgacaaactttctcaaaacagaaggccaagagcagcggttgtgtcccgaggaattcagaccggaaggtgtgtagtgtatgaagggaaccagaagacctgtgaagtctctgcctggtgccccatcgaggcagtggaagaggccccccggcctgctctcttgaacagtgccgaaaacttcactgtgctcatcaagaacaatatcgacttccccggccacaactacaccacgagaaacatcctgccaggtttaaacatcacttgtaccttccacaagactcagaatccacagtgtcccattttccgactaggagacatcttccgagaaacaggcgataatttttcagatgtggcaattcagggcggaataatgggcattgagatctactgggactgcaacctagaccgttggttccatcactgccgtcccaaatacagtttccgtcgccttgacgacaagaccaccaacgtgtccttgtaccctggctacaacttcagatacgccaagtactacaaggaaaacaatgttgagaaacggactctgataaaagtcttcgggatccgttttgacatcctggtttttggcaccggaggaaaatttgacattatccagctggttgtgtacatcggctcaaccctctcctacttcggtctggtaagagattctcttttccatgctttaggaaaatggtttggagaaggaagtgactaacgcagcgcttgtctgcattctccccaggccgctgtgttcatcgacttcctcatcgacacttactccagtaactgctgtcgctcccatatttatccctggtgcaagtgctgtcagccctgtgtggtcaacgaatactactacaggaagaagtgcgagtccattgtggagccaaagccgacattaaagtatgtgtcctttgtggatgaatcccacattaggatggtgaaccagcagctactagggagaagtctgcaagatgtcaagggccaagaagtcccaagacctgcgatggacttcacagatttgtccaggctgcccctggccctccatgacacacccccgattcctggacaaccagaggagatacagctgcttagaaaggaggcgactcctagatccagggatagccccgtctggtgccagtgtggaagctgcctcccatctcaactccctgagagccacaggtgcctggaggagctgtgctgccggaaaaagccgggggcctgcatcaccacctcagagctgttcaggaagctggtcctgtccagacacgtcctgcagttcctcctgctctaccaggagcccttgctggcgctggatgtggattccaccaacagccggctgcggcactgtgcctacaggtgctacgccacctggcgcttcggctcccaggacatggctgactttgccatcctgcccagctgctgccgctggaggatccggaaagagtttccgaagagtgaagggcagtacagtggcttcaagagtccttactgaagccaggcaccgtggctcacgtctgtaatcccagcgctttgggaggccgaggcaggcagatcacctgaggtcgggagttggagacccgcctggctaacaaggcgaaatcctgtctgtactaaaaatacaaaaatcagccagacatggtggcatgcacctgcaatcccagctactcgggaggctgaggcacaagaatcacttgaacccgggaggcagaggttgtagtgagcccagattgtgccactgctctccagcctgggaggcacagcaaactgtcccccaaaaaaaaaaaagagtccttaccaatagcaggggctgcagtagccatgttaacatgacatttaccagcaacttgaacttcacctgcaaagctctgtggccacattttcagccaaagggaaatatgctttcatcttctgttgctctctgtgtctgagagcaaagtgacctggttaaacaaaccagaatccctctacatggactcagagaaaagagattgagatgtaagtctcaactctgtccccaggaagttgtgtgaccctaggcctctcacctctgtgcctctgtctccttgttgcccaactactatctcagagatattgtgaggacaaattgagacagtgcacatgaactgtcttttaatgtgtaaagatctacatgaatgcaaaacatttcattatgaggtcagactaggataatgtccaactaaaaacaaacccttttcatcctggctggagaatgtggagaactaaaggtggccacaaattctttgacactcaagtcccccaagacctaagggttttatctcctccccttgaatatgggtggctctgattgctttatccaaaagtggaagtgacattgtgtcagtttcagatcctgatcttaagaggctgacagcttctacttgctgtcccttggaactcttgctatcggggaagccagacgccatttaaaagtctgcctatcctggccaggtgtggtggctcacacctgtaatcccagcactttgggagaccaaggcgggcggatcacttaaagtcaggagtccaagaccagactcgccaacatggtgaaaccgtatctctaataaaaatacaaaaattagctgggcatggtgcgggcacctgtagtcctagctatcaagaggctgagacaggagaaacacttgaacctgggaggtggaggttgcattgagctgagatcgtgccactgcactccaggctgggtgacagagcgagactccatctcaaaaaaaaaaaaaagaaaaaaaaaatgtctgcctatcctgagactgccctgctgtgaggaagcccaagcagtcacgtggacagtgcctgaccagccccagctttcaagccatccaagcccagtcaccaaacatgagagagaagaagccttcaggtgattctggactccactaacatatgactgataccgcatgatacatcccaagtgagaactgccccataaatccagaaaaccacattgctatcttaagtccctaagtttggggcttatttgttccacagcaacaggtaactggaacagagggcaagcctgatgaatgggcacacagactcagcccataccttccctggttctaatgttctcagggagcccggaccaaccctgggagcctcaggaacttaggtttccactggacagttctagaagggctatagaccaaatcaggtaactcaccagaccagccttggaatctatcaaatctaactgctgagctaccca

SEQ ID NO: 33 - DNA, 343 bp, Homo sapiens
ttcctgggctccctcactggcttcgtccacttaaacttttaacgtacacatgtagagagctgaaactccaggggtaacatgggacaggtcctcttgatttaatgaaaacagaagatcaactggaccgggtagcaagaaataaggcttaagaagcactgggttgggcctcaccctatgagcagggaacccaccttccccctcaacaaaagaaaagatgactttggaatgttctggattcccacagctgggaatgttcctacttttttttcagtttctctgcagaagacagcaagatgccccagggaatgtttgtgaaaaaggatgactggatgggaagcaagct

SEQ ID NO: 34 - DNA, 480 bp, Unknown
  Description of Unknown RP11-536K7.5 sequence
ctttgaatgtggcgtgtgggatctctggcgggtcatcgtcacagagctctgcaaagcaaaagaagcctattaggaactcaagaggccccaggagttctgtatgcagctgaagggtaacatcccttgggagcttttgccttgtctggctgacgccttggcaatcgcaagtgacagtgacggagagccacctcctctggaatgcatcatggaacaggtctgggagaacagagggtgcagaaagggaaaagccaggcatttgtagtcctcctgccacagatgaccagaatgacctcacccaggacaaacggcgacagatttaggaattcaagccagcttttctgactcccaggcacctgctctctcctttcgagtgttcgactccactctcagtcactcgtcactcttcccagaaggacaaatgtgcagcttctatgtcttggatttcaaataaaagacgaggaacttggtgaaatgaggtgg

SEQ ID NO: 35 - DNA, 2425 bp, Unknown
  Description of Unknown RP11-58A12.2 sequence
gagtagaaacacccctgtgatactgttcttaatattcagggaggaagaggatgatattactcccaatacagacgggtgtacaccctctgtacaccgagggtgtacacctgtctgtgaaagagttcgtaatctccagagggggagatgatattactcacaatatggtaaagaggctgtgagtccacggaggatcctcagagccagcgggggaagaggggctggctctcagtccccgcctcgcgggggtgactccccccagtgcgatgggggtcctaagagccagtgggggaagaggggctggctctgagaccccgcctcgcggggggtgcctccccgccctgtgatgggggtccgaagagccagaaggcttagaggggctggctctcagtccccgcctcgcggggggtgcctcctccccctgcgatgggggtcgtaagagccactgggggaataggggcttgctctcagtcaccgcctcgcggggggtgcctcctttcctttctacatagacacagtgacagtctgatctctctttcttttccctacagatggacacgcccccactgatattgtttttaatgcagcgtgggaggggaggatatgacacgcgatatcgcaggtagtagaaacacccctttgatagtgttcttaatattcagggaggaagaagatgatgttactcccaatacagacggatgtacaccctctgtacaccgagggtgtacacccgtctgtgaaggagttcgtaatctccagaggtggagatgatattactcacaatatggtaaacaggctgtgagtccatcgcggatcctcagagccaggtggggaagaggggctggctgtcagtcccctcctcgcggggggtgcctcccccactgctatggggatcccaagagccagtgggggaagaggtgctggctctcagtctccgcctcgcgaggtgcctccccaccctgcgatcggggtccgaagagccaggggggaagaggggctggctctcttcgtggatgattctttttccattctcaggcagttttcttttttctttctttctttttttttttttttgagactgagtcttgctctgttgcccatgctttgctcgatctcgggtgactgcaaccactgcctcccaggttcaagagattctcctgcctcagcctcctgagtagctgggactagaggcgtgtgtcaccacacccagctaatttttgtatttttagtagagatggggtttcaccatgtttgccaggatggtctctatctcctgcccttgtgatccacccacctcagcctcccaaagtgctgggattgcaggtgcgagccaccgggtccagcctctcaggcgattttcatacctgcatactctggtcactactctgttaaacagtcaaggagggtaagtattatcttcagatttccagagctctgtctctgtacagccctctcctcctcaatattctgccctatgaattctagccacattggccttcccaggctcacagttctgtcttctcaactcaggaagatctctgagttccatctgcattctttcttcctgtgctgtggcctggaaagttttctaaggtgttagggaggtcaattgtggggctagcctcatttgtttctcatctcttgaggatcactgccctttgatgcttgattccagtgattgattccctttgttgcttgagggccatagtttcatatattttgtccagtagttttgttgttttaggtcagaaagtaattttggtctctgttactctatcttggccagaagtgtaagacctaagcatttacacatcaaaatactgcacacataattttagtttaagctactttttaaaaaatctccttcattttccatttagcattctatttagggtattacattggtttttttgaaattctgttattggcagtttctattgcctatcaatcccatttaaagatagtgcatagggtattctaaaatagctgttaagcaaagagaaaattgggcctgatagggtgagaatcacagctctaatacctagagtgaccttataatgtattgtccaaaggagatatttttgacagtgaaagagggtgttgttagtaattatatcaggaccatggcctaaaccaggactatcccaggaagcctgggacatatttgtaccccatctctatttaatgcctttatacaattctttacttaattctaccagcctttattgagcctgctttctttgtctagctgagtgccacgtgctgacgtcactaagatcaatacagcaaactctgaaagatggacagagagacaggagatggtcctttataatgcagtgtgatctgtgctgcaatagagatggaagagtgttctgatcggctggaaaacaacgcacatgggaagctgtacagaaatgagtggggaaag

SEQ ID NO: 36 - DNA, 745 bp, Homo sapiens
ctgaccttgtgatccacccacctcagcctcccaaagtgctgggattgcaggtgggagccaccgggtccagcctctccggcgattttcatacctgcatactctgatcactactctgttaaacagtcaaggaggctgagtgccacgtgctgacgtcactaagatcaatacagcaaactctgaaagatggacagagagacaggagatggtcctttataatgcagtgtgatctgtgctgcaatagagttttggaagctagaagttcaaaacgaggtgttggcagcaccatgctctctctgaagatgctaggaagaatctgctccatgcctttccattcgctcctggggtttcctgcaagccctgacattccttggcttgtagatacaccaccccagtttccgcccccatcatcacatggcctcctctctgtgggtgcctctgcgttccctctattcttcttctaaggacaccgacaccagtcatagtggattaagggtccactcctaactaattacatctgcaaccaccctatttccaaataaagtcacattctaagattcctagggagaacatgaagttttgggggtgtgtggatactgttcaacctgggacatggagtaaataaatggcaagaaagattacagatgactttaagctaaagagggagatagggttaaatgtaggacttttttttttttccaggacgggagaggtttaaacatgttactacattgaataaatgaaataa

SEQ ID NO: 37 - DNA, 2139 bp, Unknown
  Description of Unknown RP13-452N2.1 sequence
gacgggcagaccacctgagctcaggagctcaagaccagcctgggcaacatggcaaaaccctgtctctactaacactataaaaagttagctgggcgtgttggtgcatgctgtagtcccagctactcaggaggctgagccacgagaatcgcttgaacctgggagatggaggttgaagtgagccaagatcgcaccattgcactccagcctggggaaaaaaaaaagccaggcctcctaatgtactcatttaaacatcacttgtgtccacctgggttcagaattgcgctggacatgggatacacaaagcgggagtggaattttcaggaagcagctgtgctgtgggtgacggtcatactttattctctaaaactctccagtttcaattggcctcttggtgccaaagcaccatggcgtgttacacctatcagatcgggcacactgccccttcttcccaacctacgagctgaaatggccagcactgcgccttccagaacccagcgtagatgcacagctgccacggaggtaggaagaatgattcgtttgccaccaaaagcccagtttctccaacaggtgaatccctaggccagcccggccccttcccacacacccctaccccgagacgggtgcagaatcagcagcggggatcatccagagactctcgcagtccacggcaggtgtcggagtgggcctcattccagagttgtggtggccgctccacctggcgagcttcgaggggactccggcaggcgctcgctgtggggtcctcccaccagtcttctcagcacaggaacggtgccttcacattggtcttccggggcttaaaagcatatcattcctgcttgctcttcagatgcaaactccccctccaaggatacctccaggttccctgccagctcacttgactcacatggcaatggtcaccgggctgcctgagcccaaatcaagcaccgtttattagggggaagctccgttatccagaaagctcatctttgaagagctacccatttcctgccaaagttgtggcgggaaggccttaggtactagaaaaccttggggaatggcaattaaaggattatcattcttacttatttgtttggataagcagcaatcacccttcggatgcttattggtgagaatatttctaaaagtgtcctgcacatttccctgctttgatgaatggaacggcagggcaggatttaactgtctgatggctacggttttgggaaagtgccagaacctccacgtcagccaccccagagccacaggcagggtatgagccacagcaatgggccccaagaaagagccccacacccaccatggtccacggaagacgctgcttactggccccgggttacctggagtcatgtgcattctccttacatcaagtgaagataatattttgtttccttcccactttttctctaacacaagccaccccagttctcccttctgacaacaggcaagtggaccctagtagaccttccagaaaagccttagagagcacccccaaatgctcacatttgtcagctgtaattccaccctcagttaaaacctgattctcaatgtgacttggccaacgcagcttgcaaagcaagaacgtcttcatctaaggttgaaaacatcagttgtttgggttgtgagaaaagaaaagcccacaaggtttccgaggcttggtgctgtttctcactgtcctcagctcctgattctgcagctggttctctctccaagcactagcaaaaccctgccctaggagcccccagactctgagagcccatgaccaaaaagaaaaggaaagccaagttggggaagaacaggggcccccaactccacagccctccactctgcccagagggcccaccctgggctgcctggaaccccctaaagttgccacccccgcaacacagtagtggggcagttcctggcagcgcctgcagcccatgggctggctctgtacccgcagccccgccaagcgtctgttatcttatttactggaatctgcacagccaggctctagctcaccggtgactaaggagctgcagccattattaccaggcagatggcagactccctaaaagcagacattaaacaataaaatgccaccacataccttgcccacaaaataaaatcaaaacaaacacctaa

SEQ ID NO: 38 - DNA, 9170 bp, Homo sapiens
accctgcaagctgcatcaggctttatcctacttgttcctttggtgaaccaggttcacttaaacttgcaaaaagatgaccgacctcttgagaagtgttgtcaccgtaattgatgttttctacaaatacaccaagcaagatggggagtgtggcacactgagcaagggtgaactaaaggaacttctggagaaagagcttcatccagttctgaagaacccagatgatccagacacagtggatgtcatcatgcatatgctggatcgagatcatgacagaagattggactttactgagtttcttttgatgatattcaagctgactatggcctgcaacaaggtcctcagcaaagaatactgcaaagcttcagggtcaaagaagcataggcgtggtcaccgacaccaagaagaagaaagtgaaacagaagaggatgaagaggatacaccaggacataaatcaggttacagacattcaagttggagtgagggagaggagcatggatatagttctgggcactcaaggggaactgtgaaatgtagacatgggtccaactccaggaggctaggaagacaaggtaatttatccagctctgggaaccaagagggatctcagaaaagataccacaggtccagctgtggtcattcatggagtggtggcaaagacagacatggttccagctctgtagaactgagagaaagaataaacaagtcacacattagcccttctagggaatctggggaggagtatgaatctggatctggatcaaacagttgggaaaggaaaggtcatggtggtctgtcatgtggattggagactagtgggcatgaatcaaactctactcagtcaagaattagagaacaaaagcttgggtctagctgttcaggttcaggagacagtgggaggcgaagtcatgcatgtggttatagcaattcaagtgggtgtggaaggccacaaaatgcttcaagttcttgtcagtcacatagatttggagggcaaggaaatcaatttagctatattcagtcaggctgtcagtcaggaattaagggaggacaaggccatggctgtgtctcaggaggtcagccctctggatgtggtcaacctgagtctaacccctgtagtcagtcctatagtcagagaggatatggagctagagaaaatggtcaaccacagaactgtggaggacaatggagaacaggctcaagtcagtcctcttgctgtggacaatatgggtctggaggtagccagtcttgtagtaatggtcaacatgaatatggttcctgtggccgcttttcaaactcttctagttcaaatgaattttccaaatgtgatcaatatgggtctggttcaagtcagtctactagctttgaacaacatggaacaggcttgagtcagtcctctgggttcgaacaacatgtatgtggctcaggtcaaacttgtggccagcatgagtctacatcaagtcaatccttgggctatgaccagcatgggtctagctcaggtaagacatctggctttggacaacatgggtctggctcaggtcagtcctctggctttggacaatgtgggtcaggctcaggtcagtcctctggctttggacagcatgggtctgtctcaggacaatcctctggttttggacagcatgggtctgtctcaggacaatcctctggttttggacaacatgagtctagatcacgtcagtctagctatggccaacatggttctggctcaagtcaatcatctggctatggccaatatgggtctagagagacatctggctttggacaacatgggttgggctcaggtcaatccactggctttggccaatatggatcgggctcaggtcagtcctctggctttggacaacatgggtctggctcaggacaatcctctggctttggacaacatgagtctagatcaggtcagtctagttatggccaacacagttctggctcaagtcagtcatctggctatggccaacatgggtctagacagacatctggctttggacaacatgggtcaggctcaagtcaatccactggctttggccaatatggatcaggctcaggtcagtcctctggctttggacaacatgtttctggctcaggacaatcctctggttttggacaacatgagtctagatcaggtcattctagctatggccaacatggttttggctcaagtcaatcatctggctatggtcaacatgggtcaagttcaggacagacatctggatttggacaacacgagttaagctcaggtcagtcttccagctttggccaacatggatcaggctcaggtcagtcctctggctttggacaacatgggtctggctcaggacaatcctctggctttggacaacatgagtctagatcaggtcagtctagctatggccaacacagttctggctcaagtcagtcatctggctatggccaacatgggtctagacagacatctggctttggacaacatgggtcaggctcaagtcaatccactggctttggccaatatggatcaggctcaggtcagtccgctggctttggacaacatgggtctggctcaggacaatcctctggctttggacagcatgagtctagatcacatcagtccagctatggccaacatggttctggctcaagtcaatcatctggctatggtcaacatgggtcaagttcgggacagacatctggctttggacaacacaggtcaagctcaggtcaatactctggctttggacaacatggatcaggctcaggtcagtccagtggctttggacaacatgggactggctcaggacaatactctggttttggacaacatgagtctagatcacatcagtctagctatggccaacatggttctggctcaagtcagtcatctggctatggtcaacatgggtcaagttcaggacagacttttggatttggacaacacaggtcaggctcaggtcaatcctctggctttggccaacatggatcaggctcaggtcagtcctctggctttggacaacatgagtcaggctcaggaaaatcctctggctttggacagcatgagtctagatcaagtcagtctaattatggccaacatggttctggctcaagtcagtcatctggctatggtcaacatgggtctagttcaggacagacaactggctttggacaacacaggtcaagctcaggccaatactcaggctttggacaacatggatcaggctcagatcagtcctctggctttggacaacatgggactggttcaggacaatcctctggttttggacaatatgagtctagatcacgtcagtctagctatggccaacatggttctggctcaagtcaatcatctggctatggtcaacatgggtcaaattcaggacagacatctggatttggacaacacaggccaggctcaggtcagtcctctggctttggccaatatggatcgggctcaggtcagtcttctggctttggacaacatgggtcaggcacaggtaaatcctctggctttgcacagcatgagtacagatcaggtcagtctagctatggccaacatggtactggctccagtcaatcatctggctgtggccaacatgagtctggctcaggtccaaccacaagttttggacagcatgtgtctggctcagacaatttctctagttctggacaacatatatctgactcaggtcagtccactggatttggccaatatggttcaggctcaggtcaatcaactggcttgggccagggtgaatctcaacaagtagagtcaggatccacagttcatgggagacaggaaactactcatggtcagacaataaataccactagacatagccagtctggtcaaggacaatccacacagacagggtccagggtaactagaagacgaagatctagccaaagtgagaacagtgacagtgaagtgcactcaaaggtctcacacagacattcagaacacattcacacacaagctggatctcactacccaaagtcaggatccacagttcgcagaagacaaggaactactcatggacagagaggagataccactagacatggccattctggtcatggacagtctacacagacaggttccagaacatctggaagacagagatttagccacagtgatgccactgacagtgaagtgcactcaggggtctcacatagaccacactcacaagaacaaactcacagccaagctggatctcaacatggagagtcagaatccacagttcatgagagacatgaaactacttatggacagacaggagaggccactggacatggccactctggtcatggacagtccacacagagagggtccaggacaactggaagaaggggatctggccatagtgagtccagtgacagtgaagtgcactcagggggctcacacagaccacaatcacaagaacaaactcatggccaagccggatctcaacatggagagtcaggatccacagttcatgggagacacggaactactcatggacagacaggagataccactagacatgcccactatcatcatggaaaatccacacagagagggtccagtacaactggaagaaggggatctggccacagtgagtccagtgacagtgaagtgcactcagggggctcgcacacacattcaggacacactcacggccaaagtggatctcaacatggagagtcagaatccataattcatgacagacacagaattactcatggacagacaggagataccactagacattcctactctggtcatgaacaaaccacacagacagggtccaggacaactggaagacagagaactagccacagtgagtccactgacagtgaagtgcactcagggggctcacacagaccacactcacgagaacacacttacggccaagccggatctcaacatgaagagccagaattcacagttcatgagagacacggaactactcatggacagataggagataccactggacattcccactctggtcatggacagtccacacagagagggtccaggacaactggaagacagagatctagccacagtgagtccagtgacagtgaagtgcactcaggggtctcacacacacatacaggacacactcatggtcaagctggatctcaacatggacagtcagaatccatagttcctgagagacatggaactactcatggacagacaggagataccactagacatgcccactatcatcatggattaaccacacagacagggtccaggactactggaagaaggggatctggccacagtgagtacagtgacagtgaagggtactcaggagtctcacatacacattcaggacacactcatggccaagccagatctcaacatggagagtcagaatccatagttcatgagagacatggaactatacatggacagacaggcgataccaccagacatgcccactctggtcatggacagtccacacagacagggtccaggaccactggaagaaggtcatctggccacagtgagtacagtgacagtgaagggcactcagggttctcacaaagaccacactcacgaggacacactcacggccaggctggatctcaacatggagagtcagaatccatagttgacgagagacatggaactactcatggacagacaggagataccagtggacattctcaatctggtcatggacagtccacacagtcaggatccagtacaactggaagaaggagatctggccacagtgagtccagtgacagtgaagtgcactcagggggctcacatacacattcaggacacacacacagccaagccaggtctcaacatggagagtcagaatccacagttcacaagagacaccaaactactcatggacagacaggagataccactgaacatggccaccctagtcatggacaaaccatacagacagggtccaggacaactggaagaaggggatctggccacagtgagtacagtgacagtgaagggccctcaggggtctcacacacacattcaggacacactcacggtcaagctggatctcactatccagagtcaggatcctcagttcatgagagacacggaactactcatggacaaacagcagataccactagacatggccactctggtcatggacagtccacacagagagggtccaggacaactggaagaagggcatctggccacagtgagtacagtgacagtgaagggcactcaggggtctcacacacacattcaggacacgctcatggccaagccggatctcaacatggagagtcaggatcctcagttcatgagagacacggaactactcatggacagacaggagataccactagacatgctcactctggtcatggacagtccacacagagagggtcaaggacagctggaagaaggggatctggccacagtgagtccagtgacagtgaagtgcactcaggggtctcacacacacattcaggacacacttatggccaagccagatctcaacatggagagtcaggatctgccattcacgggagacagggaactatacatggacagacaggagataccactagacatggccagtctggtcatggacagtccacacagacaggttccaggacaactggaagacaaagatctagtcacagtgagtccagtgatagtgaagtgcactcagaggcctcacccacacattcaggacacactcacagccaagccggatctcgacatggacagtcaggatcctcaggtcatgggagacagggaactactcatggacagacaggagataccactagacatgcccactatggttatggacaatccacacagagagggtccaggacaactggaagaaggggatctggccacagtgagtccagtgacagtgaagtgcactcatggggctcacacacacattcaggacacattcagggccaagctggatctcaacaaagacagccaggatccacagttcatgggagactggaaactactcatggacagacaggagataccactagacatggccattctggttatggacaatccacacagacaggttccagatctagtagagcaagtcattttcagtcacatagtagtgaaaggcaaaggcatggatcaagtcaggtttggaaacatggcagctatggacctgcagaatatgactatgggcacactgggtatgggccttctggtggcagcagaaaaagcatcagtaattctcacctttcatggtcaacagacagcactgcaaacaagcaactgtctagacattgacagttattttctagttctgaccttatagtatccaaagcaactaaaagaacaggaagacacagtttaaatcatgaacagtcaatggtaagttatgaacattcagttgattctcagtatcagtctcgacctattattataagaagtcaggaatctagtcatggacattctatagtaactcataaacagtcaaacaacacctatgttcaacctggatataacacagccagaagggagggatgtacacatagccagtcaaatgaccaccttggatttggccatggacaatccatatcagttcatggccattcaaaatctagttcaatcagaaaacaggaatcccatactgataacaaaaagcattcagaagattgggagaaagacactcatgagcaattaggatctaggcatgggaagttagagttcaatacaataggtatacatggatctagccagcaacatttcggagatacaacttttcatgggcaggtaagatccagcacaggttttgccagataggtattaagtcatgggccatcaagagatgcctagggtcagtctggattcagtaccaatgaaagacaagtatacagccatggccaatcaaatgatagttatgagtagtcaaatgacagcaaaagtcaaagatacattttcagtcactttcttgacagccaagaccctgcaggaattgaagagtataggtatagatattcatcaagcagtgcaaccatatgcagtgggggagacaaaggcaagagtcagagtcaagtctgtcaggaggtatcagaatatacggtgaggatgtgggcaaaaaacaaagaggctctgaggccagcggttaccatacaaaggaaagaacaggctctggttccttctgcttagatagcaacaccccactctatgaatatgtccaagaacaaaggagttattactttgaataagaagccaacataaactagcccaagataagaactaacccagagaagaaatgagacacatacatgaaattaaggtatttaacatgatctcctcttttggtagtaagggtatatgtctgttctttcattttaactatagttctgtactatattacttttgtttggtgccaggtgtttttgtaaggctccacattcattgaactccttggttagaaaatagtgaatgaggccgggcgcggtggctcacgcctgtaatcccagcactttgggaggccaagatgggtggatcacgaagtcaggagttcgagaccagcctggccaacatagtgaaatcccgtctctactaaaaatacaaaaattagctgggtgcagtagcgggcacctgtaatcctagctactcaggaggctgaggcaggagaattggttgaacccgggaggcagaggttgcagtgagccgagatcgcaccactgcactccagcctgggtgactgagcaagactccaccttgaaaaaaagaaagaaagaaaatagtgaatggaagaaaaagataaacaccatttggggctatattcagaactatataataaagagagatttgtgtggttgggaataaaattaggttttaaaaaaatttccgaatttcaaaatttggattttccagattttgttttaattaattattgacaaacctatccaaggagctaaatgacgtggtttaggagccaaaacgtctctagaaacattataacacatcccatcctgaacaaagaatgttgtcatagtttcccacatgaatattcagaatatttgcatctttttgtgtcactactggaattctgtacaattatatttaaatttattgtcatggccttctggatataggatccagaaaattgtacttcataaaaattggcaataaacatttcatcaagttaaaaaaaaaaaaaaaaaaaaaaaaaaaaaaaaaaaaaaaaaaaaaaaaa

SEQ ID NO: 39 - DNA, 2622 bp, Homo sapiens
tctgtgtcatccgccattttgtgagaagcaaggtggcctccacgtttcctgagcgtcttcttcgcttttgcctcgaccgccccttgaccacagacatgtctcgggatcggttccggagtcgtggcggtggcggtggtggcttccacaggcgtggaggaggcggcggccgcggcggcctccacgacttccgttctccgccgcccggcatgggcctcaatcagaatcgcggccccatgggtcctggcccgggccagagcggccctaagcctccgatcccgccaccgcctccacaccaacagcagcaacagccaccaccgcagcagccaccgccgcagcagccgccaccgcatcagccgccgccgcatccacagccgcatcagcagcagcagccgccgccaccgccgcaggactcttccaagcccgtcgttgctcagggacccggccccgctcccggagtaggcagcacaccaccagcctccagctcggccccgcccgccactccaccaacctcgggggccccgccagggtccgggccaggcccgactccgaccccgccgcctgcagtcacctcggcccctcccggggcgccgccacccaccccgccaagcagcggggtccctaccacacctcctcaggccggaggcccgccgcctccgcccgcggcagtcccgggcccgggtccagggcctaagcagggcccaggtccgggtggtcccaaaggcggcaaaatgcctggcgggccgaagccaggtggcggcccgggcctaagtacgcctggcggccaccccaagccgccgcgtcgaggcggcggggagccccgcgggggccgccagcaccacccgccctaccaccagcagcatcaccaggggcccccgcccggcgggcccggcggccgcagcgaggagaagatctcggactcggaggggtttaaagccaatttgtctctcttgaggaggcctggagagaaaacttacacacagcgatgtcggttgtttgttgggaatctacctgctgatatcacggaggatgaattcaaaagactatttgctaaatatggagaaccaggagaagtttttatcaacaaaggcaaaggattcggatttattaagcttgaatctagagctttggctgaaattgccaaagccgaactggatgatacacccatgagaggtagacagcttcgagttcgctttgccacacatgctgctgccctttctgttcgtaatctttcaccttatgtttccaatgaactgttggaagaagcctttagccaatttggtcctattgaaagggctgttgtaatagtggatgatcgtggaagatctacagggaaaggcattgttgaatttgcttctaagccagcagcaagaaaggcatttgaacgatgcagtgaaggtgttttcttactgacgacaactcctcgtccagtcattgtggaaccacttgaacaactagatgatgaagatggtcttcctgaaaaacttgcccagaagaatccaatgtatcaaaaggagagagaaacccctactcgttttgcccagcatggcacgtttgagtacgaatattctcagcgatggaagtctttggatgaaatggaaaaacagcaaagggaacaagttgaaaaaaacatgaaagatgcaaaagacaaattggaaagtgaaatggaagatgcctatcatgaacatcaggcaaatcttttgcgccaagatctgatgagacgacaggaagaattaagacgcatggaagaacttcacaatcaagaaatgcagaaacgtaaagaaatgcaattgaggcaagaggaggaacgacgtagaagagaggaagagatgatgattcgtcaacgtgagatggaagaccaaatgaggcgccaaagagaggaaagttacagccgaatgggctacatggatccacgggaaagagacatgcgaatgggtggcggaggagcaatgaacatgggagatccctatggttcaggaggccagaaatttccacctctaggaggtggtggtggcataggttatgaagctaatcctggcgttccaccagcaaccatgagtggttccatgatgggaagtgacatgcgtactgagcgctttgggcagggaggtgcggggcctgtgggtggacagggtcctagaggaatggggcctggaactccagcaggatatggtagagggagagaagagtacgaaggcccaaacaaaaaaccccgattttagatgtgatatttaggctttcattccagtttgttttgtttttttgtttagataccaatcttttaaattcttgcattttagtaagaaagctatctttttatggatgttagcagtttattgacctaatatttgtaaatggtctgtttgggcaggtaaaattatgtaatgcagtgtttggaacaggagaatttttttttcctttttatttctttattttttcttttttactgtataatgtccctcaagtttatggcagtgtaccttgtgccactgaatttccaaagtgtaccaatttttttttttttactgtgcttcaaataaatagaaaaatagttataaaaaaaaaaaaaaaaaaaaaaaaaaaaaaaaaaaaaaaaaaaaaaaaaaaaaaaaaaaaaaaaaaaaa

SEQ ID NO: 40 - RNA, 25 nt, Artificial Sequence
  Description of Artificial Sequence Synthetic oligonucleotide
augacagaagauuggacuuuacuga

SEQ ID NO: 41 - RNA, 27 nt, Artificial Sequence
  Description of Artificial Sequence Synthetic oligonucleotide
ucaguaaaguccaaucuucugucauga

SEQ ID NO: 42 - RNA, 25 nt, Artificial Sequence
  Description of Artificial Sequence Synthetic oligonucleotide
cugagagaaagaauaaacaagucac

SEQ ID NO: 43 - RNA, 27 nt, Artificial Sequence
  Description of Artificial Sequence Synthetic oligonucleotide
gugacuuguuuauucuuucucucaguu

SEQ ID NO: 44 - DNA, 25 bp, Artificial Sequence
  Description of Artificial Sequence Synthetic oligonucleotide
  Description of Combined DNA/RNA Molecule Synthetic oligonucleotide
agucaagaauuagagaacaaaagct

SEQ ID NO: 45 - RNA, 27 nt, Artificial Sequence
  Description of Artificial Sequence Synthetic oligonucleotide
agcuuuuguucucuaauucuugacuga

SEQ ID NO: 46 - RNA, 25 nt, Artificial Sequence
  Description of Artificial Sequence Synthetic oligonucleotide
gcagugaagguguuuucuuacugac

SEQ ID NO: 47 - RNA, 27 nt, Artificial Sequence
  Description of Artificial Sequence Synthetic oligonucleotide
gucaguaagaaaacaccuucacugcau

SEQ ID NO: 48 - DNA, 25 bp, Artificial Sequence
  Description of Artificial Sequence Synthetic oligonucleotide
  Description of Combined DNA/RNA Molecule Synthetic oligonucleotide
gcauuuuaguaagaaagcuaucutt

SEQ ID NO: 49 - RNA, 27 nt, Artificial Sequence
  Description of Artificial Sequence Synthetic oligonucleotide
aaagauagcuuucuuacuaaaaugcaa

SEQ ID NO: 50 - RNA, 25 nt, Artificial Sequence
  Description of Artificial Sequence Synthetic oligonucleotide
acgcauggaagaacuucacaaucaa

SEQ ID NO: 51 - RNA, 27 nt, Artificial Sequence
  Description of Artificial Sequence Synthetic oligonucleotide
uugauugugaaguucuuccaugcgucu

SEQ ID NO: 52 - DNA, 16 bp, Artificial Sequence
  Description of Artificial Sequence Synthetic oligonucleotide
ggtaagaaataggttg

SEQ ID NO: 53 - DNA, 15 bp, Artificial Sequence
  Description of Artificial Sequence Synthetic oligonucleotide
aacacgtctatacgc

SEQ ID NO: 54 - DNA, 21 bp, Artificial Sequence
  Description of Artificial Sequence Synthetic oligonucleotide
  Description of Combined DNA/RNA Molecule Synthetic oligonucleotide
cuuacgcugaguacuucgatt

SEQ ID NO: 55 - RNA, 19 nt, Artificial Sequence
  Description of Artificial Sequence Synthetic oligonucleotide
ucaccauuaucuucauacu

SEQ ID NO: 56 - RNA, 19 nt, Artificial Sequence
  Description of Artificial Sequence Synthetic oligonucleotide
aguaugaagauaaugguga

SEQ ID NO: 57 - RNA, 19 nt, Artificial Sequence
  Description of Artificial Sequence Synthetic oligonucleotide
ugugcuaaaauacugagau

SEQ ID NO: 58 - RNA, 19 nt, Artificial Sequence
  Description of Artificial Sequence Synthetic oligonucleotide
aucucaguauuuuagcaca